(19) United States
(12) Patent Application Publication
(10) Pub. No.: US 2012/0309939 A1
(43) Pub. Date: Dec. 6, 2012

(54) Production of Therapeutic Proteins in Photosynthetic Organisms

(75) Inventors: Beth A. Rasala, San Diego, CA (US); Rosa M. F. Cardoso, San Diego, CA (US); Machiko Muto, San Diego, CA (US); Stephen P. Mayfield, Cardiff by the Sea, CA (US); Philip A. Lee, San Diego, CA (US); Craig A. Behnke, San Diego, CA (US); Michael Mendez, San Diego, CA (US)

(73) Assignees: THE SCRIPPS RESEARCH INSTITUTE, La Jolla, CA (US); SAPPHIRE ENERGY, INC., San Diego, CA (US)

(21) Appl. No.: 13/510,229

(22) PCT Filed: Nov. 19, 2010
(86) PCT No.: PCT/US2010/057504

§ 371 Date: Aug. 15, 2012

Related U.S. Application Data
(60) Provisional application No. 61/262,826, filed on Nov. 19, 2009.

(51) Int. Cl. C12P 21/00; C07K 14/00; C12N 1/13

(52) U.S. Cl. 530/350; 435/257.2; 435/69.1

(57) ABSTRACT

The present disclosure relates to methods of expressing therapeutic proteins in photosynthetic organisms and the therapeutic proteins produced by the methods. The therapeutic proteins include high-mobility group box 1 (HMGB1) protein, fibronectin domain (10) (10FN3), fibronectin domain (14) (14FN3), interferon beta (IFNβ), proinsulin and vascular endothelial growth factor (VEGF). The photosynthetic organisms include prokaryotes such as cyanobacteria and eukaryotes such as alga and plants. Transformation of eukaryotes is preferably the plastid genome, more preferably the chloroplast genome.

### CROSS REFERENCE TO RELATED APPLICATION

This application claims the benefit of U.S. Provisional Application No. 61/262,826, filed Nov. 19, 2009, the entire contents of which are incorporated by reference for all purposes.

### STATEMENT OF RIGHTS TO INVENTIONS MADE UNDER FEDERALLY SPONSORED RESEARCH

This invention was made in part with Government support under National Institutes of Health grant A1059614. The Government may have certain rights in the invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Recombinant proteins are widely used today in many industries, including the biopharmaceutical industry, and can be expressed in bacteria, yeast, mammalian and insect cell cultures, and in transgenic plants and animals.

Since the FDA approval of recombinant insulin over 25 years ago, the class of protein-based therapeutics has grown quickly. The majority of therapeutic proteins produced today are made in bacteria (E. coli), yeast (S. cerevisiae) or mammalian cell culture (Chinese hamster ovary cells, CHO) (Demain A. L. and Vaishnav P. (2009) Biotechnol Adv 27:297-306; Walsh G. (2003) Nat Biotechnol 21:865-870; Walsh G. (2006) Nat Biotechnol 24:769-776). Other production systems under development include the yeast P. pastoris, insect cell cultures, and transgenic animals and plants.

In general, transgenic plants offer several advantages over other recombinant protein production platforms. The cost of protein production in plants is much lower than other production systems due to the low cost of (zoods and capital expenses (Dove, A. (2002) Nat Biotechnol 20:777-779). Proteins purified from plants should be free from toxins and viral agents that may be present in preparations from bacteria or mammalian cell culture. Finally, the ability to rapidly scale production in plants is difficult to achieve in other systems. Transgenic plants have been engineered, to express recombinant genes from both the nuclear and plastid (chloroplast) genomes. Nuclear expression of transgenes enables regulated and tissue-specific expression, as well as post-translational modifications. However, nuclear expression has several drawbacks to protein production; for example transgene silencing, lower yields, and the potential risk of gene flow to surrounding food crops and other native plants (Daniell H. (2006) Biotechnol 1:1071-1079). Alternatively, plastid genomes have been successfully engineered to express recombinant proteins. Advantages of chloroplast bioreactors include absence of gene silencing, targeted transgene integration by homologous recombination, expression of multiple genes from polycistrons, transgene containment via maternal inheritance of the chloroplast genome, and robust expression (Bock R. (2007) Curr Opin Biotechnol 18:100-106; Chebolu S. and Daniell H. (2009) Curr Top Microbial Immunol 332:33-54; Daniell H (2006) Biotechnol J 1:1071-1079). The chloroplast of higher plants has been shown to accumulate therapeutic proteins to 6-16% total soluble protein, vaccine antigens to as high as 31% TSP, and antimicrobial peptides to greater than 70% TSP (Chebolu S. and Daniell H. (2009) Curr Top Microbial Immunol 332:33-54; Daniell H. (2006) Biotechnol J 1:1071-1079; Oey M., et al. (2009) Plant J 57:436-445): However, the possibility of transgene escape to surrounding food crops and native plants remains, although it is greatly reduced compared to nuclear-transformed plants. While the plastid genomes in most species are maternally inherited (Hagermann R. (2004) The Sexual Inheritance of Plant Organelles, in Molecular Biology and Biotechnology of Plant Organelles pp 93-114, Netherlands: Springer Netherlands) several recent reports have demonstrated that transfer of the paternal plastid genome to pollen does occur at a low, but measurable frequency. For example, paternal inheritance has been estimated at 0.03%-0.0002% in Setaria italica (foxtail) (Shi Y., et al. (2008) Genetics 180:969-975; Wang T., et al. (2004) Theor Appl Genet 108:315-320), 0.01-0.00029% in tobacco (Ruf S., et al. 2007) Proc Natl Acad Sci USA 104:6998-7002.; Svab Z. and Maliga P. (2007) Proc Natl Acad Sci USA 104:7003-7008) and 0.0039% in Arabidopsis thaliana (Azhagiri A. K. and Maliga P. (2007) Plant J 52:817-823).

Interest in eukaryotic microalgae as an alternative platform for recombinant protein production has been gaining in recent years. Protein production in transgenic algae can offer many of the same advantages as transgenic plants, including cost, safety, and rapid scalability. In addition, microalgae can be grown, for example, in containment in enclosed bioreactors (Pulz O. (2001) Appl Microbiol Biotechnol 57:287-293), thus reducing the possibility of gene flow. Photosynthetic organisms, such as microalgae, can also be grown under varying conditions as described herein. Expression of recombinant proteins in the chloroplast of the green algae Chlamydomonas reinhardtii is well established (Mayfield S. P., et al. (2007) Curr Opin Biotechnol 18:126-133). These proteins include reporter proteins (Franklin S., et al. (2002) Plant J 30:733-744; Mayfield S. P. and Schultz J. (2004) Plant J 37:449-458; Muto M., et al. (2009) BMC Biotechnol 9:26), a large complex mammalian single chain antibody (Mayfield S. P., et al. (2003) Proc Natl Acad Sci U S A 100:438-442), more traditional single chain antibodies (Franklin S. E. and Mayfield S. P. (2005) Expert Opin Biol Ther 5:225-235), a full length monoclonal antibody (Tran M., et al. (2009) Biotechnol Bioeng) and potential vaccine antigens (Surzycki R., et al. (2009) Biologicals 37:133-138). Thus far, the psbA promoter and untranslated regions (UTRs) have been shown to support the highest levels of recombinant protein accumulation in C. reinhardtii, but only in psbA deficient strains (Manuell A. L., et al (2007) Plant Biotechnol J 5:102-412; Surzycki R., et al. (2009) Biologicals 37:133-138). Indeed, VP28 protein of the White Spot Syndrome Virus accumulated to levels as high as 20.9% total cell protein (TCP) when placed under the control of the psbA promoter and UTRs in a psbA deficient strain (Surzycki R., et al. (2009) Biologicals 37:133-138). However, because the psbA gene product D1 of photosystem II is required for photosynthesis, these transgenic algae are non-photosynthetic.

One of skill in the art would be able to choose an appropriate promoter and appropriate 5′ and 3′ UTRs as needed to drive expression of a therapeutic protein. Such promoters, regulatory or control elements, and 5′ and 3′ regions, for example, are described herein.

It would be very beneficial to be able to be express therapeutic proteins in large quantities and at a low cost. The present disclosure meets that need by providing a method to produce large quantities of a recombinant protein in a photosynthetic organism.

### SUMMARY

Provided herein are isolated polynucleotides comprising a nucleotide sequence encoding a high-mobility group box 1 (HMGB1) protein that is capable of transforming an alga. In one embodiment the nucleotide sequence is codon optimized. In another embodiment the nucleotide sequence is codon optimized for expression in a chloroplast of the alga. In yet another embodiment the nucleotide sequence is codon optimized for nuclear expression in the alga. In some embodiments the nucleotide sequence comprises a nucleic acid sequence of SEQ ID NO: 13 or SEQ ID NO: 90, or the nucleotide sequence comprises a nucleic acid sequence of SEQ ID NO: 13 or SEQ ID NO: 90 wherein the nucleic acid sequence is modified by deleting at least one nucleic acid, adding at least one nucleic acid, or replacing at least one nucleic acid, and wherein the HMGB1 protein is biologically active, or the nucleotide sequence comprises a nucleic acid sequence that has about 80% homology, about 85% homology, about 90% homology, about 95% homology, or about 99% homology to a nucleic acid sequence of SEQ ID NO: 13 or SEQ ID NO: 90, and wherein the HMGB1 protein is biologically active. In yet another embodiment the protein comprises an amino acid sequence of SEQ ID NO: 14 or SEQ ID NO: 28. In certain embodiments the polynucleotide further comprises a nucleotide sequence encoding a fusion protein fused to the 5′ end of the nucleotide sequence encoding HMGB1. In another embodiment the fusion protein is mammary-associated serum amyloid (M-SAA). In one embodiment the polynucleotide further comprises a proteolytic cleavage site between the nucleotide sequence encoding the fusion protein and the nucleotide sequence encoding HMGB1. In other embodiments the polynucleotide further comprises a nucleotide sequence (SEQ ID NO: 92) that comprises a nucleic acid sequence coding for a psbA promoter and 5′UTR, an atpA promoter (SEQ ID NO: 63) and 5′ UTR, or a psbD promoter and 5′ UTR (SEQ ID NO: 65) that is upstream of the nucleotide sequence encoding HMGB1. In other embodiments the polynucleotide further comprises a nucleotide sequence coding for a psbA 3′ UTR (SEQ ID NO: 66) or a rbcL 3′ UTR. (SEQ ID NO: 67) that is downstream of the nucleotide sequence encoding HMGB1.

Also provided herein are algae transformed with a polynucleotide comprising a nucleotide sequence encoding a HMGB1 protein. In one embodiment the nucleotide sequence is codon optimized. In another embodiment the nucleotide sequence is codon optimized for expression in the chloroplast of the alga. In other embodiments the nucleotide sequence comprises a nucleic acid sequence of SEQ ID NO: 13 or SEQ ID NO: 90. In certain embodiments the protein comprises an amino acid sequence of SEQ ID NO: 14 or SEQ ID NO: 28.

Another aspect of the disclosure provides for a method of expressing a high-mobility group box 1 (HMGB1) protein in a photosynthetic organism, comprising: a) transforming the photosynthetic organism with a polynucleotide comprising a nucleotide sequence encoding HMGB1; and b) expressing the HMGB1. In one embodiment the polynucleotide further comprises a 5′ UTR. In another embodiment, the 5′ UTR comprises a regulatory region. In yet another embodiment, the regulatory region further comprises a promoter. In other embodiments, the promoter is an endogenous promoter, the promoter is psbA or AtpA, the promoter is psbD, the promoter is a constitutive promoter, or the promoter is an inducible promoter. Where the promoter is an inducible promoter the inducible promoter can be a light inducible promoter, a nitrate inducible promoter, or a heat responsive promoter. In other embodiments the polynucleotide further comprises a nucleotide sequence (SEQ ID NO: 92) that comprises a nucleic acid sequence coding for a psbA promoter and 5′UTR, atpA promoter and 5′ UTR (SEQ ID NO: 63), or a psbD promoter and 5′ UTR (SEQ ID NO: 65) that is upstream of the nucleotide sequence encoding HMGB1. In yet another embodiment the promoter is operably linked to the expression of HMGB1. In a certain embodiment the polynucleotide further comprises a 3′ UTR. Where the polynucleotide further comprises a 3′ UTR, the 3′ UTR can be a psbA 3′ UTR (SEQ ID NO: 66) or a rbcL 3′ UTR (SEQ ID NO: 67) that is downstream of the nucleotide sequence encoding HMGB1. In another embodiment the 3′ UTR comprises a regulatory region. In some embodiments the photosynthetic organism is a prokaryote. In one embodiment the prokaryote is a cyanobacteria. In other embodiments the photosynthetic organism is a eukaryote. In another embodiment the eukaryote is a vascular plant. In another embodiment the eukaryote is a non-vascular photosynthetic organism. In a certain embodiment the non-vascular photosynthetic organism is an alga. In yet another embodiment the alga is a green alga. Where the organism is green alga, the green alga can be a Chlorophycean, a Chlamydomonas, C. reinhardtii, C. reinhardtii 137c, or a psbA deficient C. reinhardtii strain. In one embodiment the method further comprises transforming a plastid of the organism with the polynucleotide. In another embodiment the plastid is a chloroplast. In yet another embodiment the chloroplast is an algal chloroplast. In a further embodiment the nucleotide sequence encoding HMGB1 is codon-optimized to match the codon usage in a plastid of the photosynthetic organism. In an additional embodiment the polynucleotide further comprises a nucleotide sequence encoding a fusion protein fused to the 5′ end of the nucleotide sequence encoding HMGB1. In one embodiment the fusion protein is mammary-associated serum amyloid (M-SAA). In another embodiment the polynucleotide further comprises a proteolytic cleavage site between the nucleotide sequence encoding the fusion protein and the nucleotide sequence encoding HMGB1. In yet another embodiment the polynucleotide further comprises a nucleotide sequence encoding a purification tag downstream of the nucleotide sequence encoding HMGB1. In a certain embodiment the tag is a FLAG-tag. In a further embodiment the tag comprises an amino acid sequence DYKDDDDKS (SEQ ID NO: 60). In one embodiment the nucleotide sequence encoding HMGB1 encodes for human HMGB1. In another embodiment the nucleotide sequence encoding HMGB1 encodes for a rodent HMGB1. In other embodiments the rodent protein comprises an amino acid sequence of SEQ ID NO: 77 or the rat protein comprises an amino acid sequence of SEQ ID NO: 78. In further embodiments the nucleotide sequence comprises a nucleic acid sequence of SEQ ID NO: 13 or SEQ ID NO: 90. In additional embodiments the nucleotide sequence comprises a nucleic acid sequence of SEQ ID NO: 13 or SEQ ID NO: 90 wherein the nucleic acid sequence is modified by deleting at least one nucleic acid, adding at least one nucleic acid, or replacing at least one nucleic acid, and wherein the HMGB1 protein is biologically active, or the nucleotide sequence comprises a nucleic acid sequence that has about 80% homology, about 85% homology, about 90% homology, about 95% homology, or about 99% homology to a nucleic acid sequence of SEQ ID NO: 13 or SEQ ID NO: 90, and wherein the HMGB1 protein is biologically active. In yet other embodiments the protein comprises an amino acid sequence of SEQ ID NO: 14 or SEQ ID NO: 28. In one embodiment the nucleotide sequence encoding HMGB1 is codon-optimized to match the nuclear codon usage of the photosynthetic organism. In other embodiments the transformation is by particle bombardment. In some embodiments the HMGB1 is expressed at at least 0.5%, at least 1%, at least 1.5%, at least 2.0%, at least 2.5%, or at least 3.0% of total soluble protein. Also provided are HMGB1 proteins made by the methods disclosed herein.

The present disclosure encompasses a method of expressing a high-mobility group box 1 (HMGB1) protein in an alga, comprising: a) transforming the alga with a polynucleotide comprising a nucleotide sequence encoding HMGB1; and b) expressing the HMGB1. Also provided are isolated photosynthetic organisms comprising a polynucleotide comprising a nucleotide sequence encoding a high-mobility group box 1 protein (HMGB1), wherein the photosynthetic organism is capable of expressing the HMGB1 protein. In another embodiment the polynucleotide further comprises a 5′ UTR. In an additional embodiment the 5′ UTR comprises a regulatory region. In one embodiment the regulatory region further comprises a promoter. Where a regulatory regions comprises a promoter, the promoter can be an endogenous promoter, a psbA, AtpA, or psbD promoter, a constitutive promoter, or an inducible promoter. Where the promoter is an inducible promoter, the inducible promoter can be a light inducible promoter, a nitrate inducible promoter, or a heat responsive promoter. In other embodiments the polynucleotide further comprises a nucleotide sequence (SEQ ID NO: 92) that comprises a nucleic acid sequence coding for a psbA promoter and 5′UTR, an atpA promoter and 5′ UTR (SEQ ID NO: 63), or a psbD promoter and 5′ UTR (SEQ ID NO: 65) that is upstream of the nucleotide sequence encoding HMGB1. In yet another embodiment the promoter is operably linked to the expression of HMGB1. In one embodiment the polynucleotide further comprises a 3′ UTR. In some embodiments the polynucleotide further comprises a psbA 3′ (SEQ ID NO: 66) or a rbcL 3′ UTR (SEQ ID NO: 67) that is downstream of the nucleotide sequence encoding HMGB1. In another embodiment the 3′ UTR comprises a regulatory region. In some embodiments the photosynthetic organism is a prokaryote. Where the organism is a prokaryote, the prokaryote can be a cyanobacteria. In some embodiments the photosynthetic organism is a eukaryote. In one embodiment the eukaryote is a vascular plant. In another embodiment the eukaryote is a non-vascular photosynthetic organism. In still another embodiment the non-vascular photosynthetic organism is an alga, in one embodiment the alga is green alga. Where the organism is a green alga, the green alga can be a Chlorophycean, a Chlamydomonas, C. reinhardtii, C. Reinhardtii 137c, or a psbA deficient C. reinhardtii strain. In one embodiment a plastid of the organism is transformed with the polynucleotide. In another embodiment the plastid is a chloroplast. In a certain embodiment the chloroplast is an algal chloroplast. In another embodiment the nucleotide sequence encoding HMGB1 is codon-optimized to match the codon usage in a plastid of the photosynthetic organism. In yet another embodiment the polynucleotide further comprises a nucleotide sequence encoding a fusion protein fused to the 5′ end of the nucleotide sequence encoding HMGB1. In a further embodiment the fusion protein is mammary-associated serum amyloid (M-SAA). On another embodiment the polynucleotide further comprises a proteolytic cleavage site between the nucleotide sequence encoding the fusion protein and the nucleotide sequence encoding HMGB1. In one embodiment the polynucleotide further comprises a nucleotide sequence encoding a purification tag downstream of the nucleotide sequence encoding HMGB1. In yet another embodiment the tag is a FLAG-tag. In a further embodiment the FLAG-tag comprises an amino acid sequence DYKDDDDKS (SEQ ID NO: 60). In one embodiment the nucleotide sequence encoding HMGB1 encodes for human HMGB1. In another embodiment the nucleotide sequence encoding HMGB1 encodes for a rodent HMGB1. In other embodiments the rodent protein comprises an amino acid sequence of SEQ ID NO: 77 or the rat protein comprises an amino acid sequence of SEQ ID NO: 78. In certain embodiments the nucleotide sequence comprises a nucleic acid sequence of SEQ ID NO: 13 or SEQ ID NO: 90. In other embodiments the nucleotide sequence comprises a nucleic acid sequence of SEQ ID NO: 13 or SEQ ID NO: 90 wherein the nucleic acid sequence is modified by deleting at least one nucleic acid, adding at least one nucleic acid, or replacing at least one nucleic acid, and wherein the HMGB1 protein is biologically active, or the nucleotide sequence comprises a nucleic acid sequence that has about 80% homology, about 85% homology, about 90% homology, about 95% homology, or about 99% homology to a nucleic acid sequence of SEQ ID NO: 13 or SEQ ID NO: 90, and wherein the HMGB1 protein is biologically active. In some embodiments the protein comprises an amino acid sequence of SEQ ID NO: 14 or SEQ ID NO: 28. In one embodiment the nucleotide sequence encoding HMGB1 is codon-optimized to match the nuclear codon usage of the photosynthetic organism. In other embodiments the HMGB1 is expressed at at least 0.5%, at least 1%, at least 1.5%, at least 2.0%, at least 2.5%, or at least 3.0% of total soluble protein.

Also provided are methods of expressing a therapeutic protein in a photosynthetic organism, comprising: a) transforming the photosynthetic organism with a polynucleotide comprising a nucleotide sequence encoding the therapeutic protein, wherein the therapeutic protein is fibronectin domain 10 (10FN3), fibronectin domain 14 (14FN3), interferon beta, proinsulin, or vascular endothelial growth factor (VEGF); and b) expressing the therapeutic protein. In one embodiment the polynucleotide further comprises a 5′ UTR. In another embodiment the 5′ UTR comprises a regulatory region. In yet another embodiment the regulatory region further comprises a promoter. Where the regulatory region comprises a promoter, the promoter can be an endogenous promoter, a psbA or AtpA promoter, a psbD promoter, a constitutive promoter, or an inducible promoter. Where the promoter is an inducible promoter, the inducible promoter can be alight inducible promoter, a nitrate inducible promoter, or a heat responsive promoter. In further embodiments the polynucleotide further comprises a nucleotide sequence (SEQ ID NO: 92) that comprises a nucleic acid sequence coding for a psbA promoter and 5′UTR, on atpA promoter and 5′ UTR, (SEQ ID NO: 63), or a psbD promoter and 5′ UTR (SEQ ID NO: 65) that is upstream of the nucleotide sequence encoding the therapeutic protein. In another embodiment the promoter is operably linked to the expression of the therapeutic protein. In yet another embodiment the polynucleotide further comprises a 3′ UTR. In some embodiments the polynucleotide further comprises a psbA 3′ UTR (SEQ ID NO: 66) or a rbcL 3′ UTR (SEQ ID NO: 67) that is downstream of the nucleotide sequence encoding the therapeutic protein. In one embodiment the 3′ UTR comprises a regulatory region. In some embodiments the photosynthetic organism is a prokaryote. In another embodiment the prokaryote is a cyanobacterium. In other embodiments the photosynthetic organism is a eukaryote. In one embodiment the eukaryote is a vascular plant. In other embodiments the eukaryote is a non-vascular photosynthetic organism. In one embodiment the non-vascular photosynthetic organism is an alga. In yet another embodiment the alga is green alga. Where the organism is a green alga, the green alga can be a Chlorophycean, a Chlamydomonas, C. reinhardtii, C. Reinhardtii 137c, or a psbA deficient C. Reinhardtii strain. In a certain embodiment the method further comprises comprising transforming a plastid with the polynucleotide. In one embodiment the plastid is a chloroplast. In a farther embodiment the chloroplast is an algal chloroplast. In one embodiment the nucleotide sequence encoding the therapeutic protein is codon-optimized to match the codon usage in a plastid of the photosynthetic organism. In an additional embodiment the polynucleotide further comprises a nucleotide sequence encoding a fusion protein fused to the 5′ end of the nucleotide sequence encoding the therapeutic protein. In a further embodiment the fusion protein is mammary-associated serum amyloid (M-SAA). In yet another embodiment the polynucleotide further comprises a proteolytic cleavage site between the nucleotide sequence encoding the fusion protein and the nucleotide sequence encoding the therapeutic protein. In some embodiments the nucleotide sequence encoding a fusion protein encodes for mammary-associated serum amyloid (M-SAA) and the nucleotide sequence encoding the therapeutic protein encodes for 14FN3, 10FN3, or VEGF. In an additional embodiment the polynucleotide further comprises a nucleotide sequence encoding a purification tag. In a further embodiment the tag is a FLAG-tag. In another embodiment the FLAG-tag comprises the amino acid sequence DYKDDDDKS (SEQ ID NO: 60). In certain embodiments the nucleotide sequence encoding the therapeutic protein encodes a human protein. In other embodiments the nucleotide sequence encoding the therapeutic protein encodes an animal protein. In further embodiments the nucleotide sequence comprises a nucleic acid sequence of SEQ ID NO: 3, SEQ ID NO: 5, SEQ ID NO: 7, SEQ ID NO: 9, SEQ ID NO: 11, SEQ ID NO: 82, SEQ ID NO: 83, SEQ ID NO: 84, SEQ ID NO: 86, or SEQ ID NO: 88, or the nucleotide sequence comprises a nucleic acid sequence of SEQ ID NO: 3, SEQ ID NO: 5, SEQ ID NO: 7, SEQ ID NO: 9, SEQ ID NO: 11, SEQ ID NO: 82, SEQ ID NO: 83, SEQ ID NO: 84, SEQ ID NO: 86, or SEQ ID NO: 88 wherein the nucleic acid sequence is modified by deleting at least one nucleic acid, adding at least one nucleic acid, or replacing at least one nucleic acid, and wherein the therapeutic protein is biologically active, or the nucleotide sequence comprises a nucleic acid sequence that has about 80% homology, about 85% homology, about 90% homology, about 95% homology, or about 99% homology to a nucleic acid sequence of SEQ ID NO: 3, SEQ ID NO: 5, SEQ ID NO: 7, SEQ ID NO: 9, SEQ ID NO: 11, SEQ ID NO: 82, SEQ ID NO: 83, SEQ ID NO: 84, SEQ ID NO: 86, or SEQ ID NO: 88, and wherein the therapeutic protein is biologically active. In other embodiments the therapeutic protein comprises an amino acid sequence of SEQ ID NO: 4, SEQ ID NO: 6, SEQ ID NO: 8, SEQ ID NO: 10, SEQ ID NO: 12, SEQ ID NO: 18, SEQ ID NO: 20, SEQ ID NO: 22, SEQ ID NO: 24, or SEQ ID NO: 26. In yet another embodiment the nucleotide sequence encoding the therapeutic protein is codon-optimized to match the nuclear codon usage of the photosynthetic organism. In some embodiments the transformation is by particle bombardment. In other embodiments the therapeutic protein is expressed at at least 0.5%, at least 1%, at least 1.5%, at least 2.0%, at least 2.5%, or at least 3.0% of total soluble protein. Also provided are therapeutic proteins made by the methods disclosed herein.

The present disclosure also provides isolated photosynthetic organisms comprising a polynucleotide comprising a nucleotide sequence encoding a therapeutic protein, wherein the therapeutic protein is fibronectin domain 10 (10FN3), fibronectin domain 14 (14FN3), interferon beta, proinsulin, or vascular endothelial growth factor (VEGF), and wherein the photosynthetic organism is capable of expressing the therapeutic protein. In one embodiment the polynucleotide further comprises a 5′ UTR. In another embodiment the 5′ UTR comprises a regulatory region. In yet another embodiment the regulatory region further comprises a promoter. Where the regulatory regions comprises a promoter, the promoter can be an endogenous promoter, a psbA or AtpA promoter, a psbD promoter, a constitutive promoter, or an inducible promoter. Where the promoter is an inducible promoter, the inducible promoter can be light inducible promoter, a nitrate inducible promoter, or a heat responsive promoter. In other embodiments the polynucleotide further comprises a nucleotide sequence (SEQ ID NO: 92) that comprises a nucleic acid sequence coding for a psbA promoter and 5′UTR, an atpA promoter and 5′ UTR (SEQ ID NO: 63), or a psbD promoter and 5′ UTR (SEQ ID NO: 65) that is upstream of the nucleotide sequence encoding the therapeutic protein. In another embodiment the promoter is operably linked to the expression of the therapeutic protein. In yet another embodiment the polynucleotide further comprises a 3′ UTR. In other embodiments the polynucleotide further comprises a psbA 3′ UTR (SEQ ID NO: 66) or a rbcL 3′ UTR (SEQ ID NO: 67) that is downstream of the nucleotide sequence encoding the therapeutic protein. In a certain embodiment the 3′ UTR comprises a regulatory region. In still another embodiment the photosynthetic organism is a prokaryote. In one embodiment the prokaryote is a cyanobacterium. In other embodiments the photosynthetic organism is a eukaryote. In yet another embodiment the eukaryote is a vascular plant. In a certain embodiment the eukaryote is a non-vascular photosynthetic organism. In one embodiment the non-vascular photosynthetic organism is an alga. In another embodiment the alga is green alga. Where the organism is a green alga, the green alga can be a Chlorophycean, a Chlamydomonas, C. reinhardtii. C. Reinhardtii 137c, or a psbA deficient C. reinhardtii strain. In another embodiment a plastid of the organism is transformed with the polynucleotide. In one embodiment the plastid is a chloroplast. In yet another embodiment the chloroplast is an algal chloroplast. In one embodiment the nucleotide sequence encoding the therapeutic protein is codon-optimized to match the codon usage in a plastid of the photosynthetic organism. In another embodiment the polynucleotide further comprises a nucleotide sequence encoding a fusion protein fused to the 5′ end of the nucleotide sequence encoding the therapeutic protein. In a further embodiment the fusion protein is mammary-associated serum amyloid (M-SAA). In yet another embodiment the polynucleotide further comprises a proteolytic cleavage site between the nucleotide sequence encoding the fusion protein and the nucleotide sequence encoding the therapeutic protein. In some embodiments the nucleotide sequence encoding a fusion protein encodes for mammary-associated serum amyloid (M-SAA) and the nucleotide sequence encoding the therapeutic protein encodes for 14FN3, 10FN3, or VEGF. In another embodiment the polynucleotide further comprises a nucleotide sequence encoding a purification tag. In a further embodiment the tag is a FLAG-tag. In yet another embodiment the FLAG-tag comprises the amino acid sequence DYKDDDDKS (SEQ ID NO: 60). In some embodiments the nucleotide sequence encoding the therapeutic protein encodes a human protein. In other embodiments the nucleotide sequence encoding the therapeutic protein encodes an animal protein, In yet other embodiments the nucleotide sequence comprises a nucleic acid sequence of SEQ ID NO: 3, SEQ ID NO: 5, SEQ ID NO: 7, SEQ ID NO: 9, SEQ ID NO: 11, SEQ ID NO: 82, SEQ ID NO: 83, SEQ ID NO: 84, SEQ ID NO: 86, or SEQ ID NO: 88, or the nucleotide sequence comprises a nucleic acid sequence of SEQ ID NO: 3, SEQ ID NO: 5, SEQ ID NO: 7, SEQ ID NO: 9, SEQ ID NO: 11, SEQ ID NO: 82, SEQ ID NO: 83, SEQ ID NO: 84, SEQ ID NO: 86, or SEQ ID NO: 88 wherein the nucleic acid sequence is modified by deleting at least one nucleic acid, adding at least one nucleic acid, or replacing at least one nucleic acid, and wherein the therapeutic protein is biologically active, or the nucleotide sequence comprises a nucleic acid sequence that has about 80% homology, about 85% homology, about 90% homology, about 95% homology, or about 99% homology to a nucleic acid sequence of SEQ ID NO: 3, SEQ ID NO: 5, SEQ ID NO: 7, SEQ ID NO: 9, SEQ ID NO: 11, SEQ ID NO: 82, SEQ ID NO: 83, SEQ ID NO: 84, SEQ ID NO: 86, or SEQ ID NO: 88, and wherein the therapeutic protein is biologically active. In still other embodiments the therapeutic protein comprises an amino acid sequence of SEQ ID NO: 4, SEQ ID NO: 6, SEQ ID NO: 8, SEQ ID NO: 10, SEQ ID NO: 12, SEQ ID NO: 18, SEQ ID NO: 20, SEQ ID NO: 22, SEQ ID NO: 24, or SEQ ID NO: 26. In an additional embodiment the nucleotide sequence encoding the therapeutic protein is codon-optimized to match the nuclear codon usage of the photosynthetic organism. In other embodiments the therapeutic protein is expressed at at least 0.5%, at least 1%, at least 1.5%, at least 2.0%, at least 2.5%, or at least 3.0% of total soluble protein.

Provided herein are novel methods of expressing a therapeutic protein of interest in a photosynthetic organism, comprising transforming the photosynthetic organism with at least one polynucleotide comprising a nucleotide sequence encoding the therapeutic protein of interest, wherein the therapeutic protein is one or more of fibronectin domain 10 (10FN3), fibronectin domain 14 (14FN3), proinsulin, vascular endothelial growth factor (VEGF), or high-mobility group box 1 or amphoterin (HMGB1), and expressing the therapeutic protein of interest. The polynucleotide can further comprise a 5′ UTR. In one embodiment, the 5′ UTR comprises a regulatory region. In another embodiment, the regulatory region further comprises a promoter. In other embodiments, the promoter is an endogenous promoter, psbA, AtpA, a constitutive promoter, or an inducible promoter. In the case of an inducible promoter, the promoter may be a light inducible promoter, nitrate inducible promoter, or a heat responsive promoter. In yet another embodiment, the promoter is operably linked to the expression of the therapeutic protein. In another embodiment, the polynucleotide further comprises a 3′ UTR. In yet another embodiment, the 3′ UTR comprises a regulatory region. In another embodiment, the photosynthetic organism is a prokaryote. In the case of a prokaryote, the prokaryote may be a cyanobacteria. In yet another embodiment, the photosynthetic organism is a eukaryote. In the case of a eukaryote, the eukaryote may be a vascular plant or a non-vascular photosynthetic organism. In the case of a non-vascular photosynthetic organism, the non-vascular photosynthetic organism may be an alga. In the case of an alga, the alga may be a green alga. In the case of a green alga, the green alga may be a Chlorophycean or a Chlamydomonas. In the case of a Chlamydomonas, the Chlamydomonas may be C. reinhardtii or C. Reinhardtii 137c. In certain embodiments, the method further comprising transforming a plastid with the at least one polynucleotide. In the case of a plastid, the plastid may be a chloroplast. In the case of a chloroplast, the chloroplast may be an algal chloroplast. In one embodiment, the nucleotide sequence encoding the therapeutic protein of interest is codon-optimized to match the codon usage in the plastid of the photosynthetic organism. In another embodiment, the polynucleotide further comprises a nucleotide sequence encoding a fusion partner fused to the amino-terminus of the nucleotide sequence encoding the therapeutic protein. In the case of a fusion partner, the fusion partner may be encoded by the nucleotide sequence for mammary-associated serum amyloid (M-SAA) protein. In yet another embodiment, the polynucleotide further comprises a proteolytic cleavage site between the nucleotide sequence for mammary-associated serum amyloid (M-SAA) protein and the nucleotide sequence encoding the therapeutic protein. In another embodiment, the at least one polynucleotide further comprises a nucleotide sequence encoding a purification tag. In the case of a purification tag, the purification tag may be a FLAG-tag. In one embodiment, the therapeutic protein is a mammalian protein. In another embodiment, the therapeutic protein is a human protein. In other embodiments, the at least one polynucleotide may comprise the nucleotide sequence of one or more of SEQ ID NO: 3, SEQ ID NO: 5, SEQ ID NO: 7 SEQ ID NO: 9, SEQ ID NO: 11, SEQ ID NO: 13, SEQ ID NO: 82, SEQ ID NO: 83, SEQ ID NO: 84, SEQ ID NO: 86, SEQ ID NO: 88, or SEQ ID NO: 90.

In other embodiments, the therapeutic protein may comprise the amino acid sequence of one or more of SEQ ID NO: 4, SEQ ID NO: 6, SEQ ID NO: 8, SEQ ID NO: 10, SEQ ID NO: 12, SEQ ID NO: 14, SEQ ID NO: 18, SEQ ID NO: 20, SEQ ID NO: 22, SEQ ID NO: 24, SEQ ID NO: 26, or SEQ ID NO: 28. In yet another embodiment, the nucleotide sequence encoding the therapeutic protein of interest is codon-optimized to match the codon usage of the photosynthetic organism. In one embodiment, the transformation is by particle bombardment.

Another aspect provides therapeutic proteins made by any of the novel methods described above.

Still another aspect provides an isolated photosynthetic organism comprising at least one polynucleotide comprising a nucleotide sequence encoding a therapeutic protein of interest, wherein the therapeutic protein is one or more of fibronectin domain 10 (10FN3), fibronectin domain 14 (14FN3), proinsulin, vascular endothelial growth factor (VEGF), or high-mobility group box 1 or amphoterin (HMGB1), and wherein the photosynthetic organism is capable of expressing the therapeutic protein of interest. In one embodiment, the polynucleotide further comprises a 5′ UTR. In yet another embodiment, the 5′ UTR comprises a regulatory region. In yet another embodiment, the regulatory region further comprises a promoter. In other embodiments, the promoter may be an endogenous promoter, psbA, AtpA, a constitutive promoter, or an inducible promoter. In the case of an inducible promoter, the inducible promoter may be a light inducible promoter, nitrate inducible promoter, or a heat responsive promoter. In one embodiment, the promoter is operably linked to the expression of the therapeutic protein. In yet another embodiment, the polynucleotide further comprises a 3′ UTR. In certain embodiments, the 3′ UTR comprises a regulatory region. In another embodiment, the photosynthetic organism is a prokaryote. In the case of a prokaryote, the prokaryote may be a cyanobacterium.

In one embodiment, the photosynthetic organism is a eukaryote. In the case of a eukaryote, the eukaryote may be a vascular plant or a non-vascular photosynthetic organism. In the case of a non-vascular photosynthetic organism, the non-vascular photosynthetic organism may be an alga. In the case of an alga, the alga may be a green alga. In the case of a green alga, the green alga may be a Chlorophycean or a Chlamydomonas. In the case of a Chlamydomonas, the Chlamydomonas may be C. reinhardtii or C. Reinhardtii 137c. In another embodiment, a plastid of the organism is transformed with the at least one polynucleotide. In the case of a plastid, the plastid may be a chloroplast. In the case of a chloroplast, the chloroplast may be an algal chloroplast. In yet another embodiment, the nucleotide sequence encoding the therapeutic protein of interest is codon-optimized to match the codon usage in the plastid of the photosynthetic organism. In certain embodiments, the polynucleotide further comprises a nucleotide sequence encoding a fusion partner fused to the amino-terminus of the nucleotide sequence encoding the therapeutic protein. In the case of a fusion partner, the fusion partner may be encoded by the nucleotide sequence for mammary-associated serum amyloid (M-SAA) protein. In yet another embodiment, the polynucleotide further comprises a proteolytic cleavage site between the nucleotide sequence for mammary-associated serum amyloid (M-SAA) protein and the nucleotide sequence encoding the therapeutic protein. In another embodiment, the at least one polynucleotide further comprises a nucleotide sequence encoding a purification tag. In the case of a purification tag, the purification tag may be a FLAG-tag. In one embodiment, the therapeutic protein is a mammalian protein. In another embodiment, the therapeutic protein is a human protein. In other embodiments, the at least one polynucleotide may comprise the nucleotide sequence of one or more of SEQ ID NO: 3, SEQ ID NO: 5, SEQ ID NO: 7, SEQ ID NO: 9, SEQ ID NO: 11, SEQ ID NO: 13, SEQ ID NO: 82, SEQ ID NO: 83, SEQ ID NO: 84, SEQ ID NO: 86, SEQ ID NO: 88, or SEQ ID NO: 90. In other embodiments, the therapeutic protein may comprise the amino acid sequence of one or more of SEQ ID NO: 4, SEQ ID NO: 6, SEQ ID NO: 8, SEQ ID NO: 10, SEQ ID NO: 12, SEQ ID NO: 14, SEQ ID NO: 18, SEQ ID NO: 20, SEQ ID NO: 22, SEQ ID NO: 24, SEQ ID NO: 26, or SEQ ID NO: 28. In yet another embodiment, the nucleotide sequence encoding the therapeutic protein of interest is codon-optimized to match the codon usage of the photosynthetic organism.

### BRIEF DESCRIPTION OF THE DRAWINGS

These and other features, aspects, and advantages of the present invention will become better understood with regard to the following description, appended claims and accompanying figures where:

FIG. 1 shows the introduction of the recombinant genes into the Chlamydomonas reinhardtii chloroplast genome. Schematic diagram of transformation vectors used, including relevant restriction sites. (FIGS. 1A and B) pD1—KanR: Replacement of the endogenous psbA gene with the gene of interest (FIG. 1A), or with the gene of interest fused to the C-terminus of M-SAA (Manuell A. L., et al (2007) Plant Biotechnol J 5:402-412) (FIG. 1B). The kanamycin resistance gene aphA6 under the control of the atpA promoter and 5′ UTR is genetically linked to the gene of interest. The solid portions flanking the gene of interest and resistance gene correspond to regions of the chloroplast genome used for homologous recombination between the insertion plasmid and the C. reinhardtii chloroplast genome. (FIG. 1C) Schematic diagram of p322 (Franklin S., et al. (2002) Plant J 30:733-744) used to transform the genes of interest under the control of the atpA promoter and 5′ UTR and the rbcL 3′ UTR into the intergenic region between psbA exon 5 and the 5S rRNA locus (FIG. 13) (Barnes D., et al. (2005) Mol Genet Genomics 274:625-636). The nucleic acid sequences that were used for cloning are SEQ ID NOs: 1, 3, 5, 7, 9, 11, and 13. All recombinant proteins were C-terminally fused to the 1× FLAG-tag sequence (DYKDDDDKS) (SEQ ID NO: 60) for western blotting and purification.

FIG. 2 shows the identification of gene integration and isolation of homoplasmic strains. PCR was done using whole cell lysates. G: Gene specific PCR to show the presence of the corresponding recombinant gene in the transformants. The following primers were used: PsbA forward reverse primer (SEQ ID NO: 35); AtpA forward reverse primer (SEQ ID NO: 36); gene specific reverse primer EPO (SEQ ID NO: 37); gene specific reverse primer 10FN3 (SEQ ID NO: 38); gene specific reverse primer 14FN3 (SEQ ID NO: 39); gene specific reverse primer interferon beta (SEQ ID NO: 40); gene specific reverse primer proinsulin (SEQ ID NO: 41); gene specific reverse primer VEGF (SEQ ID NO: 42); and gene specific reverse primer for HMGB1 (SEQ ID NO: 43). H: PCR to show homoplasmicity of the clones. Each reaction contains two sets of primers (SEQ ID NOs: 33 and 34), one that amplifies an internal control gene (16S rRNA) to demonstrate that the PCR reactions worked, and can be seen in all lanes (H-C), and the other primer set amplifies the region of the genome that was targeted for integration (psbA: SEQ ID NOs: 29 and 30; (atpA: SEQ ID NOs: 31 and 32) and thus the parent strain shows a band whereas homoplasmic transformants do not (H-I). The ladder shown is 1 kb+ from Invitrogen (Invitrogen, USA). The order of lanes from left to right are: 1 (EPO); 2 (10FN3); 3 (14FN3); 4 (interferon β); 5 (proinsulin); 6 (VEGF); and 7 (HMGB1).

FIG. 3 shows the accumulation of recombinant proteins in transgenic lines. Strains were grown and harvested for western blotting. Equal amounts of total protein were loaded in each lane (20 μg for A and C, 40 μg for B). Western blots were probed with anti-FLAG antibody conjugated to Horse Radish Peroxidase. (A) Protein accumulation in strains when the corresponding genes were expressed from the psbA promoter and UTRs. (B) Protein accumulation when the corresponding genes were expressed from the atpA promoter and UTRs. (C) Protein accumulation of the SAA fusion proteins from the psbA promoter. Short exposure times were routinely used for (A) and (C), while much longer exposure times (several hours) were required to visualize the bands in (B).

FIG. 4 shows the quantitation of protein accumulation. Percent total soluble protein was determined for 14FN3, VEGF, and HMGB1 by loading 10 or 20 μg of soluble lysate from expression strains onto a SDS-PAGE gel alongside a serial dilution of highly pure HMGB1. Western blots were performed using anti-FLAG-HRP antibody.

FIG. 5 shows the analysis of mRNA levels for psbA and atpA constructs. mRNA levels of the seven recombinant genes under the control of the psbA and atpA promoters are represented (psbA promoter is black and atpA promoter is cross hatched). Fold change determined using the Pfaffl method (Pfaffl M. W. (2001) Nucleic Acids Res 29:e45) to take into account differing PCR efficiencies with the different gene-specific primer pairs used. Forward and reverse primers used are as follows: EPO: SEQ ID NOs: 44 and 45; 10FN3: SEQ ID NOs: 46 and 47; 14FN3: SEQ ID NOs: 48 and 49; interferon beta: SEQ ID NOs: 50 and 51; proinsulin: SEQ ID NOs: 52 and 53; VEGF: SEQ ID NOs: 54 and 55; and HMGB1: SEQ ID NOs: 56 and 57.

atpA-EPO yielded the lowest level of mRNA, so all mRNA levels were calculated as fold change relative/compared to atpA-EPO.

FIGS. 6A, 6B, and 6C show affinity purification of algal-expressed therapeutic proteins. Top panel depicts coomassie staining and the boxed bottom panel depicts western blotting of the 14FN3 (FIG. 6A), VEGF (FIG. 6B), and HMGB1 (FIG. 6C) purifications. Lanes from left to right are the following fractions: insoluble fraction (Ins), total soluble protein (TSP), column flow through (Flow), and the eluate (Elu). Equal volumes of Ins, TSP and Flow are loaded per lane. 3 μg of purified protein is loaded on each coomassie gel, while 500 ng of Elu is loaded on each western blot. Right panels correspond to the MADLI-TOF MS results for each purified protein; the y axis represents counts and the x axis represents mass (m/z), The ladder used was Biorad Precision Plus protein marker (Biorad, USA).

FIG. 7 shows the bioactivity of VEGF. (A) VEGF ELISA: concentration of intact VEGF in the purified protein was assessed by comparison with bacteria-derived VEGF in a sandwich ELISA. (B) Competitive binding to the VEGF receptor was assayed by detecting binding of a fixed concentration of algal VEGF to VEGFR-coated wells in the presence of varying concentrations of bacteria-derived VEGF.

FIGS. 8A and 8B show the bioactivity of HMGB1. Graph of the results from the fibroblast chemotaxis assay, as measured by the number of mouse (FIG. 8A) or pig (FIG. 8B) fibroblasts migrating towards the indicated chemokine is shown. Bioactivity of algal-expressed HMGB1 (Scripps) compared to commercial HMGB1 (Bio3), and to the controls mouse VEGF (A) or pig PDGF (B). Data represents mean and standard deviations of each treatment condition.

FIG. 9 shows the expression analysis of unique clones. Six homoplasmic clones for 14FN3, VEGF, and HMGB1 expressed from the psbA promoter and UTRs (pD1-KanR construct) were tested for recombinant protein accumulation by western blot. A small aliquot for each was scraped off a Tris-acetate-phosphate (TAP)/agar plate into lysis buffer, lysed, and 20 μls of total soluble protein was loaded onto SDS-page gel and blotted. Equal volumes but not equal amounts of protein were analyzed.

FIG. 10 shows the accumulation of recombinant proteins under photosynthetic conditions following reintroduction of psbA. 80 μg of total soluble protein from the indicated strains were separated by SDS-PAGE and subjected to western blotting with mouse anti-FLAG-AP. (A) TSP from the psbA knockout strain expressing 14FN3 under control of the psbA promoter and 5′ and 3′ UTRs is shown in lane 5. The psbA cDNA under control of the psbD promoter and 5′ UTR (SEQ ID NO: 65) and the psbA 3′ UTR (SEQ ID NO: 66) was reintroduced into a silent site in the genome. Two independent and homoplasmic lines were grown under photosynthetic conditions (high salt medium (HSM), lane 1 and 2) or heterotrophically (TAP, lanes 3 and 4). (B) Similarly, TSP from the strain expressing HMGB1 under control of the psbA promoter and UTRs in the psbA null background is shown in lane 1. Two independent and homoplasmic lines expressing HMGB1 plus pshbD::psbA were grown autotrophically (HSM, lane 2 and 3) or heterotrophically (TAP, lanes 4 and 5).

FIG. 11 shows the integrity of the isolated total RNA. Total RNA from transgenic algae expressing the transgene under control of the atpA promoter and 5′ UTR (a) or the psbA, promoter and 5′ UTR (p). RNA was subjected to agarose gel electrophoresis and stained with ethidium bromide.

FIG. 12 shows a VEGF receptor-binding assay. ELISA analysis of VEGF-R coated plates demonstrate that algal-expressed VEGF is capable of binding to human VEGF receptor in a dose-dependent manner with similar affinity compared to bacterial-expressed VEGF. The y axis represents absorbance at 450 and the x axis represents relative concentration of the protein added. R&D is commercially available bacterial derived VEGF from R&D Systems (Minneapolis, US). R6 is algal-expressed VEGF.

FIG. 13 is a vector map of p322. This transformation vector integrates a transgene runder the control of the atpA promoter and 5′ UTR and rbcL 3′ UTR between exon 5 of psbA and the 5S rRNA. The backbone of the vector is pBluescript KS+.

FIG. 14 is a vector map of cloning vector pD1-KanR.

FIG. 15 shows a BamHI-HindIII (4.8 kb) insert from BamHI 11/12 that was cloned into the BamHI and HindIII site of pUC18 to make p228. The 4.8 kb fragment comprises a 16S rRNA and a 23Ss rRNA (5′ end). pUC18 comprises a selectable marker for ampicillin resistance. The C. reinhardtii 16S rRNA gene comprises a spr-u-1-6-2 mutation, an A->G change at bp 1123, which causes loss of an AatII restriction site and confers high level spectinomycin resistance. (See Harris et al., Genetics 123, 281-292 (1989); Newman et al., Genetics 126, 875-888 (1990)).

FIG. 16A is a phylogenetic tree showing evolutionary relationships of the HMGB1 gene between different species. The phylogenetic tree was constructed according to the calculation of the best match for the selected sequences. The order of species from top to bottom is: Homo sapiens, Pan troglodytes, Macaca mulatta, Mus musculus, Rattus norvegicus, Canis familiaris, Equus caballus, Bos taurus, Sus scrofa, Xenopus laevis, Danio rerio, and Saimo salar.

FIG. 16B shows functional domains within the HMGB1 amino acid sequence. The full-length HMGB1 contains two homogenous domains (A- and B-box) and an acidic C-terminal tail. The B-box is associated with its properties relevant to proinflammatory activity and the receptor for advanced glycation end products (RAGE) binding, while the A-box is a specific antagonist by which it inhibits the proinflammatory properties of HMGB1. The C-terminal acidic tail is required for the transcription stimulatory function of HMGB1.

FIG. 17 is a HMGB1 amino acid sequence alignment showing evolutionary conservation between diverse species. Sequence homology: black, 100% identical; gray, >75% identical; arrows indicate >50% identical; and white, 0% identical.

FIG. 18 shows HMGB1's affinity for a number of different DNA structures.

FIG. 19 shows HMGB1's interacting proteins from several DNA repair pathways (nucleotide excision repair, mismatch repair, base excision repair, and DNA double-strand break repair).

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

As used in this specification and the appended claims, the singular forms “a”, “an” and “the” include plural reference unless the context clearly dictates otherwise.

Endogenous

An endogenous nucleic acid, nucleotide, polypeptide, or protein as described herein is defined in relationship to the host organism. An endogenous nucleic acid, nucleotide, polypeptide, or protein is one that naturally occurs in the host organism.

Exogenous

An exogenous nucleic acid, nucleotide, polypeptide, or protein as described herein is defined in relationship to the host organism. An exogenous nucleic acid, nucleotide, polypeptide, or protein is one that does not naturally occur in the host organism or is a different location in the host organism.

Nucleotide and amino acid sequences (SEQ ID NOs: 1-92) are useful in the embodiments disclosed herein. If a stop codon is not present at the end of a coding sequence, one of skill in the art would know to insert nucleotides encoding for a stop codon (TAA, TAG, or TGA) at the end of the nucleotide sequence. If an initial start codon (Met) is not present in an amino acid sequence, one of skill in the art would be able to include, at the nucleotide level, an initial ATG, so that the translated polypeptide would have an initial Met.

Additionally, if an enzyme restriction site was needed for cloning purposes at either the 5′ end and/or the 3′ end of a coding sequence, one of skill in the art would be able to engineer in the appropriate restriction site(s).

One of skill in the art would also know how to “link” together sequences, for example, a FLAG-tag with a TEV-FLAG tag. One example of such a linker is the amino acid sequence SGGGGS.

Also included in SEQ ID NOs: 1-91 are primer sequences and affinity tags useful in the embodiments disclosed herein.

The present disclosure relates to novel methods of expressing a therapeutic protein in a photosynthetic organism, and the therapeutic protein produced by the novel method. Also provided are photosynthetic organisms comprising the therapeutic protein.

To examine the versatility of photosynthetic organisms for the production of human protein therapeutics, different recombinant genes, all encoding current or potential human protein therapeutics were expressed using as an exemplary photosynthetic organism, algae.

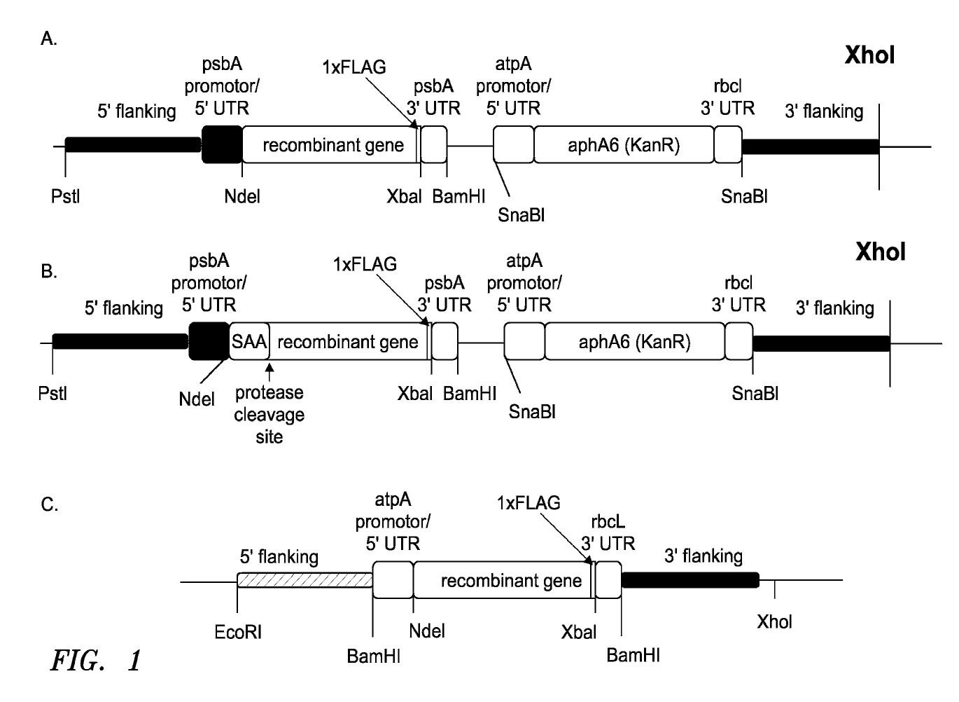

Using three different expression vectors (FIG. 1A, 1B, and 1C), production of four of the seven genes tested was achieved. Of the seven proteins chosen, greater than 50% expressed at levels sufficient for commercial production. Three proteins accumulated to above 2% of total soluble protein, levels sufficient for easy purification, when the genes were driven from the psbA promoter in a psbA deficient strain. The atpA promoter also drove expression of the same three proteins, but to significantly lower levels. A carboxy-terminal fusion of each of the seven therapeutic proteins to the M-SAA protein resulted in the accumulation of the same three proteins that expressed with the psbA promoter alone, as well as an additional recombinant protein that did not express on its own. All of the algal chloroplast-expressed proteins were found to be soluble. Two of the proteins were purified and assayed for bioactivity using standard assays, and both were found to have similar activity to the same protein produced in a more traditional expression system. Together, these results demonstrate how the algal chloroplast is a viable platform for the expression of a diverse set of recombinant human therapeutic proteins.

The proteins chosen for this study are a diverse group of proteins, some of which are already produced as therapeutics, and others that have the potential to become therapeutic proteins in the future (Table 1).

Table 1 shows the codon adaptive index (CAI) values for the native human sequences (SEQ ID NOs: 68 to 74) and the codon optimized sequences compared against the C. Reinhardtii chloroplast codon usage table.

| | | | CAI of |
|---|---|---|---|
| | corresponding | CAI of | codon optimized |
| gene | amino acids | native sequence | sequence |
| EPO | as 28-193 | 0.25 | 0.83 |
| 10FN3 | as 1447-1540 | 0.39 | 0.80 |
| (NP997639.1) | | | |
| 14FN3 | as 1723-1811 | 0.35 | 0.81 |
| (NP997639.1) | | | |
| Inf β | aa 23-187 | 0.33 | 0.84 |
| Proinsulin | as 25-110 | 0.24 | 0.77 |
| VEGF isoform | aa 27-147 | 0.30 | 0.79 |
| 121 | | | |
| HMGB1 | aa 2-185 | 0.40 | 0.82 |
| (NP002119.1) | | | |

SEQ ID NOs: 1 and 2 depict the codon-optimized nucleotide sequence of erythropoietin (EPO) that was used for cloning and the resulting amino acid sequence, respectively.

SEQ ID NOs: 3 and 4 depict the codon-optimized nucleotide sequence of fibronectin domain 10 (10FN3) that was used for cloning and the resulting amino acid sequence, respectively.

SEQ ID NOs: 5 and 6 depict the codon-optimized nucleotide sequence of fibronectin domain 14 (14FN3) that was used for cloning and the resulting amino acid sequence, respectively.

SEQ ID NOs: 7 and 8 depict the codon-optimized nucleotide sequence of interferon beta that was used for cloning and the resulting amino acid sequence, respectively.

SEQ ID NOs: 9 and 10 depict the codon-optimized nucleotide sequence of proinsulin that was used for cloning and the resulting amino acid sequence, respectively.

SEQ ID NOs: 11 and 12 depict the codon-optimized nucleotide sequence of vascular endothelial growth factor (VEGF) that was used for cloning and the resulting amino acid sequence, respectively.

SEQ ID NOs: 13 and 14 depict the codon-optimized nucleotide sequence of high-mobility group box 1 or amphoterin (HMGB1) that was used for cloning and the resulting amino acid sequence, respectively.

It would be beneficial to be able to be produce therapeutic proteins, such as those listed above, in large quantities and at low cost. The first protein is human erythropoietin (EPO) without its signal peptide, a human hormone produced by both the liver and kidney that regulates red blood cell production and also plays an important role in the response of the brain to neural injury and wound healing (Haroon Z. A., et al. (2003) Am J Pathol 163:993-1000; Siren A. L., et al. (2001) Acta Neuropathol 101:271-276). Recombinant EPO is currently produced in mammalian cells and is used in the treatment of anemia (Eschbach J. W., et al. (1989) Ann Intern Med 111:992-1000; Jelkmann W (2007) Eur J Haematol 78:183-205). The second and third proteins are domains ten and fourteen of human fibronectin, respectively. Fibronectin is an extracellular matrix glycoprotein that functions in cell adhesion, migration, growth and differentiation (Pankov R. and Yamada K. M. (2002) J Cell Sci 115:3861-3863).

Fibronectin is comprised of multiple domains and can bind to integrins as well as collagen, fibrin and heparin sulfate proteoglycans. The tenth human fibronectin type III domain (10FN3) is a stable 10 kDa beta-sandwich subunit that has potential to be an antibody mimic (monobody) (Garcia-Ibilcieta D., et al. (2008) Biotechniques 44:559-562; Koide A. and Koide S. (2007) Methods Mol Biol 352:95-109). The fourteenth human fibronectin type III domain (14FN3) is part of the heparin-II/VEGF binding domain (Wijelath E. S., et al. (2006) Circ Res 99:853-860) and is in development as a framework for antibody mimics. The fourth protein is human interferon β1. Interferons improve the integrity of the blood brain barrier and are used in the treatment of Multiple Sclerosis (MS) (Murdoch D. and Lyseng-Williamson K. A. (2005) Drugs 65:1295-1312). A one month supply of interferon β, Avonex (Biogen Idec) or Rebif (EMD Serono and Pfizer), can cost anywhere from $1,600 to more than $2,000 USD (McCormack P. L. and Scott L. J. (2004) CNS Drugs 18:521-546). The fifth protein used in this study is human proinsulin (without its signal peptide), a hormone that regulates blood sugar level. Insulin is used in the treatment of type I diabetes, has a multi-billion dollar market dominated by Eli Lilly (e.g. Humulin) and Novo Nordisk, and was the first genetically engineered drug approved by the FDA. The sixth protein is human vascular endothelial growth factor (VEGF) isoform 121 (without its signal peptide). Patients suffering from pulmonary emphysema have decreased levels of VEGF in their pulmonary arteries. VEGF also has the potential to be a treatment for erectile dysfunction (Strong T. D., et al. (2008) Asian J Androl 10:14-22) and depression Warner-Schmidt J. L. and Duman R. S. (2008) Curr Opin Pharmacol 8:14-19). The seventh and final protein is high mobility group protein B1 (HMGB1) which mediates a number of important functions involved in wound healing including endothelial cell activation, stromagenesis, recruitment and activation of innate immune cells, and dendritic cell maturation (Sun N. K. and Chao C. C. (2005) Chang Gung Med J 28:673-682). It has also been suggested that HMGB1 has the potential to enhance the effectiveness of some anti-cancer therapies if co-administered (Dong Xda E., et al. (2007) J Immunother 30:596-606; Krynetskaia N., et al. (2008) Mol Pharmacol 73:260-269).

HMGB1

The high mobility group BL (HMGB1) protein (previously known as HMG1, or amiphoterin), is a member of the high mobility group family of proteins. This family is separated into three groups: the HMGA (formerly HMG-I/Y) proteins, so named because they contain an A-T hook domain that binds selectively to the minor groove of AT-rich DNA; the HMGB proteins, which contain a DNA-binding B box domain that binds distorted or non-B DNA structures with high affinity and induces severe bends in the DNA; and HMGN proteins (previously named HMG-14/17), which contain a nucleosome binding domain responsible for binding to nucleosomes (Bustin, M., Trends Biochem. Sci. (2001) 26(3):152-153). All of these proteins are so-called “architectural transcription factors” because they act by binding the DNA in a structure dependent manner, and modify transcriptional regulation and chromatin structure (Grosschedl, R., et at., Trends. Genet. (1994) 10(3):94-100). A number of comprehensive reviews have been written about the activity of the HMG family of proteins (Reeves, R. and Adair, J. E., DNA Repair (Amst) (2005) 4(8):926-938; Hock, R., et al., Trends Cell Biol. (2007) 17(2):72-79).

This family of non-histone, chromatin associated nuclear proteins was discovered as specific regulators of gene expression more than 35 years ago (Goodwin, G. H., et al., Eur. J. Biochem. (1973) 38:14-19). HMG proteins are constitutively expressed in the nucleus of eukaryotic cells. They were confirmed to be involved in DNA organization and regulation of transcription. They share functional motifs that bind specific DNA structures and induce conformational changes without specificity for target sequences. They have such structural characteristics as transcripts with long AT-rich 3′ untranslated regions and highly negatively charged carboxy-terminals (Bustin, M., Mol. Cell. Biol. (1999) 19: 5237-5246).

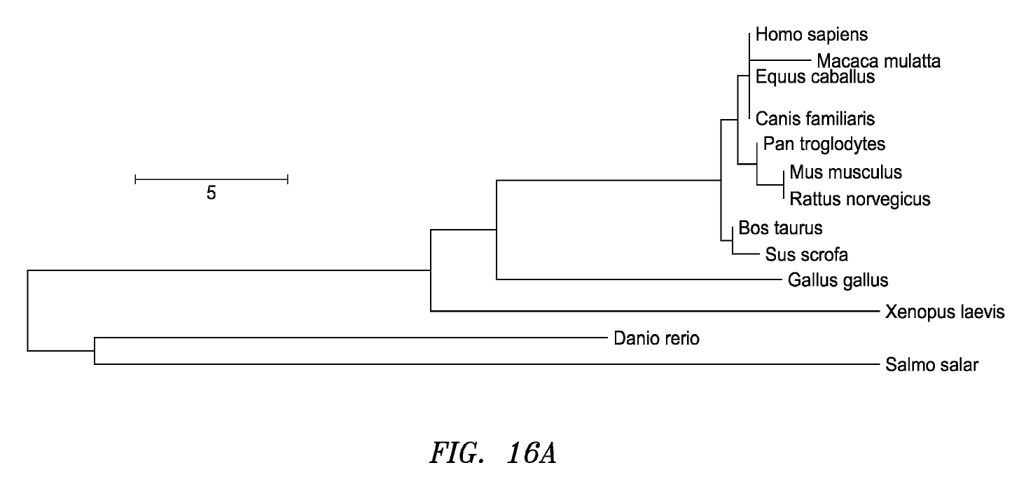

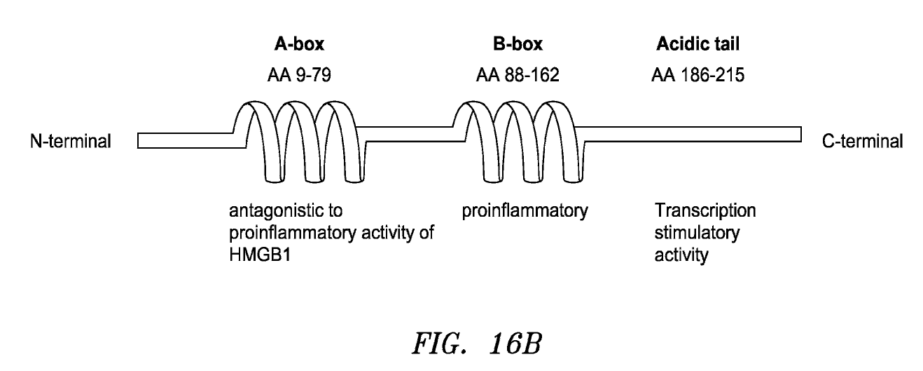

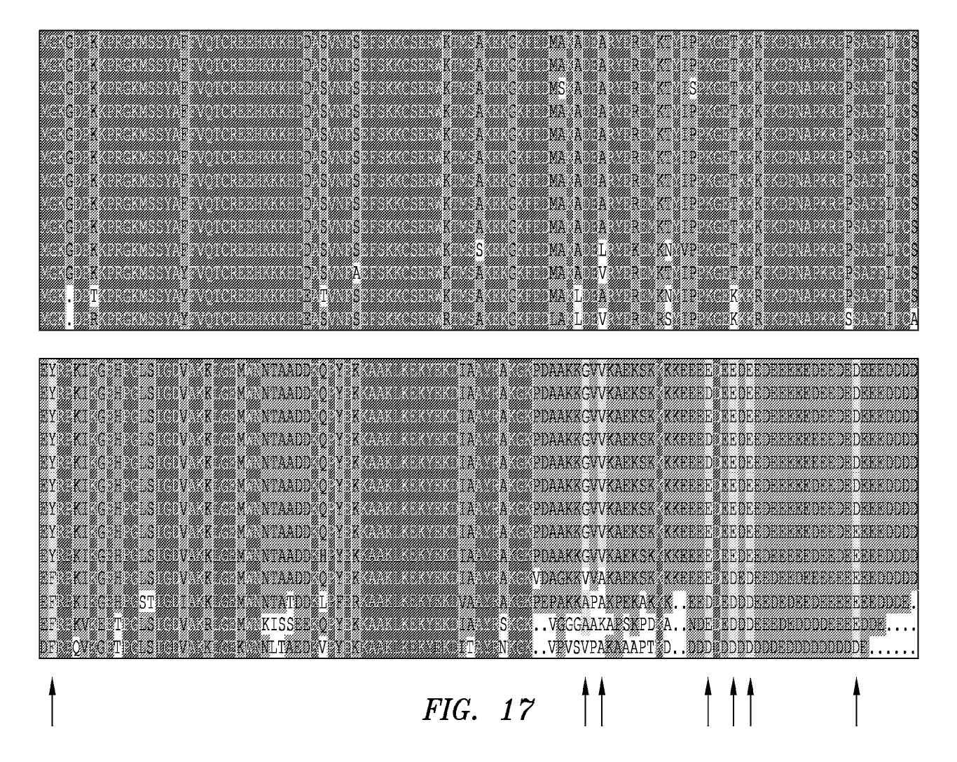

HMGB1 probably originated more than 500 million years ago before the split between the animal and plant kingdoms (FIG. 16A). It is among the most evolutionarily conserved pro-teins in the eukaryotic kingdom and shares 100% amino acid (AA) identity between mice and rats, and 99% AA identity between rodents and humans. The species listed are from top to bottom are as follows: Homo sapiens, Pan troglodytes, Macaca mulatta, Mus musculus, Rattus norvegicus, Canis familiaris, Equus caballus, Bos taurus, Sus scrofa, Gallas gallus, Xenopus laevis, Danio rerio, and Salmo salar. Exemplary amino acid sequences showing high sequence identity are SEQ ID NOs: 77, 78, and 79.

HMGB1 has a concentration of about 106 molecules per cell and is constitutively expressed in quiescent cells, and a large “pool” of performed HMGB1 is stored in the nucleus (Bustin, M., Mol. Cell. Biol. (1999) 19: 5237-5246). As a nuclear protein, HMGB1 is implicated in diverse cellular functions, including the regulation of nucleosomal structure and stability, and transcription factors binding to their cognate DNA sequences (Bustin, M., Mol. Cell. Biol. (1999) 19:5237-5246; Bianchi, M. E., et al., Science (1989) 243:1056-1059; Hill, D. A. and Reeves, R., Nucleic Acids Res. (1997) 25:3523-3531; Hill, D. A., et al., Nucleic Acids Res (1999) 27:2135-2144; Locker, D., et al., Mol Biol (1995) 246:243-247; Stros, M. and Reich, J., Eur. J. Biochem. (1998) 251:427-434). The binding activity of HMGB1 to DNA is regulated by the two 80-amino acid DNA binding domains, the A-box and B-box, with each structurally represented as three α-helices in a characteristic L-shaped fold (Weir, H. M., et al., EMBO J. (1993) 12:1311-1319) (FIG. 16B). In addition to the A- and B-box, there is an acidic tail in the C-terminal of HMGB1. The C-terminal acidic tail is important for the transcription stimulatory function of HMGB1 (Weir, H. M., et al., EMBO J. (1993) 12:1311-1319; Landsman, D. and Bustin, M., Bioessays (1993) 15:539-546; Ueda, T., et al., Biochemistry (2004) 43:9901-9908; Wang, H., et al., Am. J. Respir. Crit. Care Med. (2001) 164:1768-1773). The two boxes bind to the minor groove of chromatin thus modifying the DNA architecture. This facilitates the binding of regulatory proteins of various transcription factors to their cognate sequences, including the steroid/nuclear hormones progesterone (Onate, S. A., et al., Mol. Cell. Biol. (1994) 14:3376-3391) and estrogen (Verrier, C. S., et al., Mol. Endocrinol. (1997) 11:1009-1019; Zhang, C. C., et al., Mol. Endocrinol. (1999) 13:632-643) HOX proteins (Zappavigna, V., et al., EMBO J. (1996) 15:4981-4991), p53, homeobox-containing proteins, recombination activating gene 1/2 (RAG 1/2) proteins and transcription factor II B (Sutrias-Grau, M., et al., J. Biol. Chem. (1999) 274:1628-1634).

HMGB1 is a small (25 kDa) protein who's myriad of intracellular and extracellular roles are mediated by its relatively simple domain structure. As discussed briefly above, HMGB1 contains 3 domains: the A and B box domains, which are characteristic of the HMGB family members and are responsible for binding to and bending of DNA; and a C-terminal 30 amino acid acidic tail (Thomas, J. O. and Travers, A. A., Trends Biochem. Sci. (2001) 26(3):167-174). These domains allow HMGB1 to bind DNA in a structure-specific fashion, and this ability is responsible for its intracellular roles. It has been shown that HMGB1 preferentially binds to non-canonical DNA structures and damaged DNA, and thus affects the repair of damaged DNA. In addition, HMGB1 can be post-translationally modified, particularly acetylated, and this affects both its ability to bind and bend the DNA (Pasheva, E., et al., Biochemistry (2004) 43(10):2935-2940; Ugrinova, I., et al., Biochemistry (2001) 40(48):14655-14660), as well as its subcellular localization (Bonaldi, T., et al., Embo J. (2003) 22(20):5551—-5560).

The high mobility group protein B1 (HMGB1) is a highly abundant protein with roles in several cellular processes, including chromatin structure and transcriptional regulation, as well as an extracellular role in inflammation (Lange, S. S, and Vasquez, K. M., Mol. Carcinog. (2009) 48(7):571-580). HMGB1's most thoroughly defined function is as a protein capable of binding specifically to distorted and damaged DNA, and its ability to induce further bending in the DNA once it is bound. This characteristic in part mediates its function in chromatin structure (binding to the linker region of nucleosomal DNA and increasing the instability of the nucleosome structure) as well as in transcription (bending promoter DNA to enhance the interaction of transcription factors).

HMGB1 is believed to have a role in the nucleotide excision repair (NER) pathway, in both “repair shielding” and “repair enhancing”. In addition, HMGB1 has a role in the mismatch repair (MMR), non-homologous end-joining (NHEJ), and V(D)J recombination pathways, as well as in the base excision repair (BER) pathway. HMGB1 may also be involved in DNA repair, in the context of chromatin.

As an architectural nuclear factor, HMGB1 is capable of binding to the linker region of nucleosomal DNA (Schroter, H. and Bode, J., Eur. J. Biochem. (1982) 127(2):429-436; Nightingale, K., et al., Embo J. (1996) 15(3):548-561) and it competes with histone H1 to modify the dynamics of chromatin structure (Catez, F, et al., Mol. Cell. Biol. (2004) 24(10):4321-4328). In addition, HMGB1 acts as a transcriptional cofactor, enhancing the association of the TBP-TATA complex with the transcriptional start site (Das, D. and Scovell, W. M., J. Biol. Chem. (2001) 276(35):32597-32605). Perhaps the best demonstration of HMGB1's critical role in transcription came in 1999, when Calogero et al. developed HMGB1 knockout mice (Calogero, S., et al., Nat. Genet. (1999) 22(3):276-280), which die shortly after birth from hypoglycemia, and exhibit improper regulation of the glucocorticoid receptor. HMGB1 has also been shown to interact with and enhance the activities of a number of transcription factors implicated in cancer development, including p53 (Jayaraman, L, et al., Genes Dev. (1998) 12(4):462-472), retinoblastoma protein (RB) (Jiao, Y., et al., Acta Pharmacol. Sin. (2007) 28(12):1957-1967) and the estrogen receptor (ER) (Melvin, V. S., et al., J. Biol. Chem. (2004) 279(15):14763-14771). These functions of HMGB1 are mediated by its ability to bind to DNA and induce further bends into the DNA.

In addition to these intracellular roles, in 1999 Wang et al. (Wang, H., et al., Science (1999) 285(5425):248-251) demonstrated that HMGB1 is secreted from activated macrophages, and is a pathogenic mediator in the inflammatory disease. The study of HMGB1's extracellular roles in inflammation has greatly expanded since this discovery, and HMGB1 is now being targeted for therapeutic intervention to treat sepsis and rheumatoid arthritis (Ulloa, L. and Messmer, D., Cytokine Growth Factor Rev. (2006) 17(3): 189-201). In addition, when present in the extracellular matrix, HMGB1's binding to the receptor for advanced glycation end-products (RAGE) may mediate tumor growth, invasion and metastasis (Ellerman, J. E., et al., Clin. Cancer Res. (2007) 13(10):2836-2848).

The therapeutic potential of HMGB1-targeting agents for the treatment of sepsis is discussed in Wang, H., et al., Expert Reviews in Molecular Medicine (2008) 10:1-20, and Wang, H., et al., Shock (2009) 32(4):348-357. Anti-HMGB1 therapies are being developed to treat inflammatory diseases. For example, the role of HMGB1 as a potential mediator of cystic fibrosis airway inflammation is discussed in Gaggar, A., et al., The Open Respiratory Medical Journal (2010) 4:32-38.

Therapeutic proteins are used for the treatment or prevention of a disease or disorder. Therapeutic proteins can be mammalian proteins, for example, human proteins. The therapeutic proteins can be used for veterinarian care of for human care. Therapeutic proteins can be used for to treat companion, domestic, exotic, wildlife and production animals. The therapeutic proteins can be involved in, for example, cell signaling and signal transduction. Examples of therapeutic proteins are antibodies, transmembrane proteins, growth factors, enzymes, or structural proteins. The therapeutic protein can be a protein found in an animal, or in a human, or a derivative of a protein found in an animal or in a human.

The nucleotide sequence encoding a therapeutic protein of interest can be the naturally occurring or wild-type sequence or can be a modified sequence. Types of modifications include, the deletion of at least one nucleic acid, the addition of at least one nucleic acid, or the replacement of at least one nucleic acid. One skilled in the art would know how to make modifications to the nucleotide sequence.

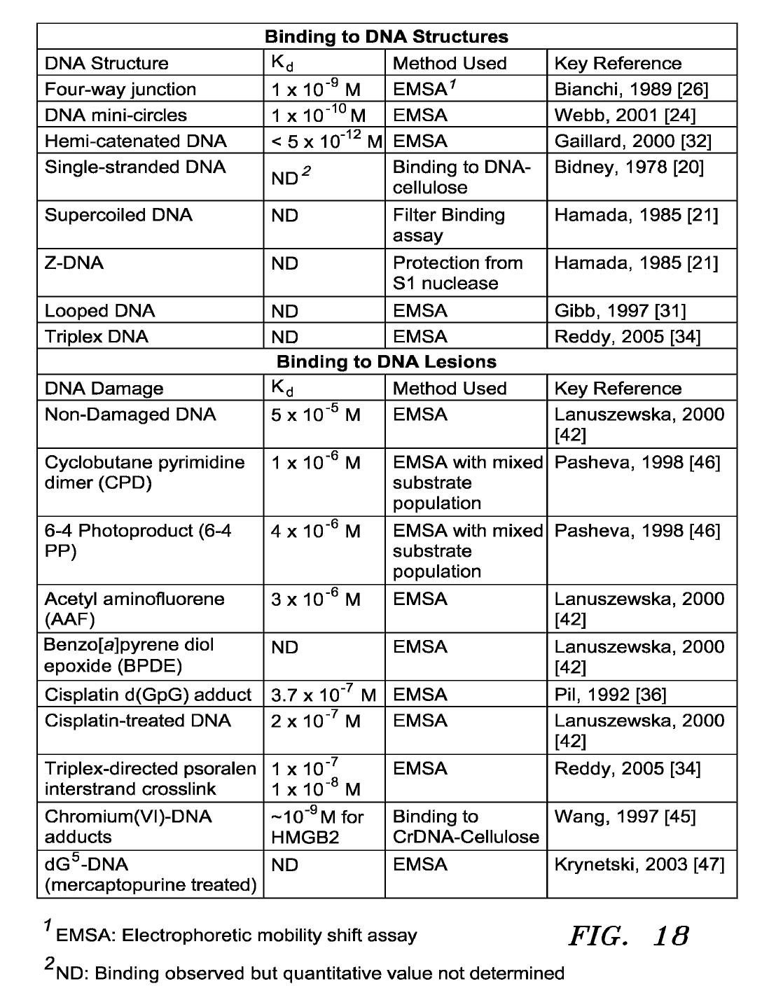

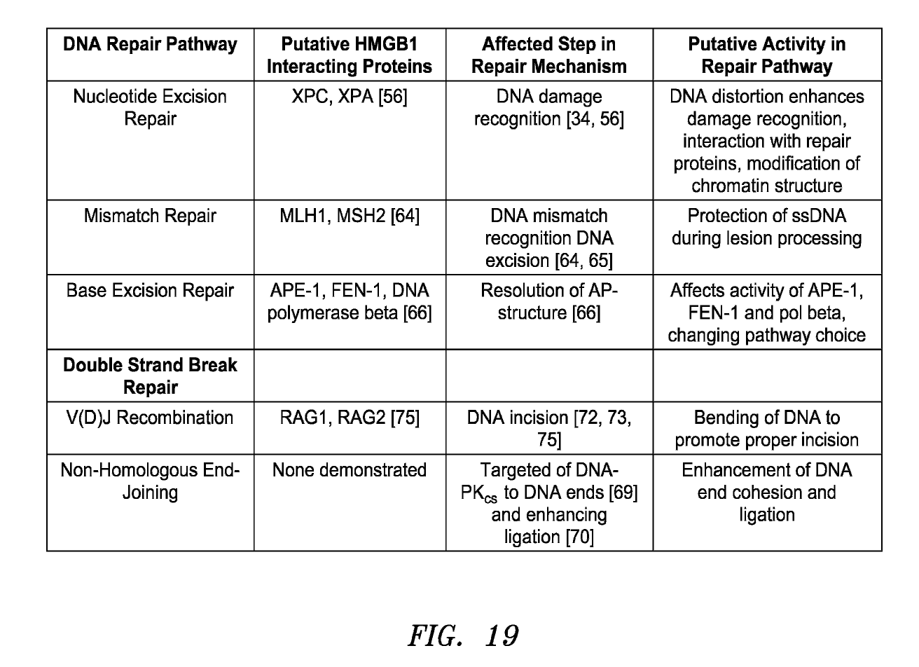

For example, a nucleotide sequence encoding for a HMGB1 protein can be modified by deleting at least one nucleic acid, adding at least one nucleic acid, or replacing at least one nucleic acid, wherein the HMGB1 protein retains its biological activity. Biological activity can be, for example, the ability of the protein to signal an immune response, for example, wound repair. An exemplary assay to test for biological activity is the chemotaxis assay described herein. Examples of HMGB1's roles in DNA repair are shown in FIGS. 18 and 19.

Host Cells or Host Organisms

A host cell can contain a polynucleotide encoding a therapeutic protein of the present disclosure. In some embodiments, a host cell is part of a multicellular organism. In other embodiments, a host cell is cultured as a unicellular organism.

Host organisms can include any suitable host, for example, a microorganism. Microorganisms which are useful for the methods described herein include, for example, photosynthetic bacteria (e.g., cyanobacteria), non-photosynthetic bacteria (e.g., E. coli), yeast (e.g., Saccharomyces cerevisiae), and algae (e.g., microalgae such as Chlamydomonas reinhardtii).

Examples of host organisms that can be transformed with a therapeutic protein of interest (for example, a polynucleotide that encodes a high-mobility group box 1 protein or a VEGF protein) include vascular and non-vascular organisms. The organism can be prokaryotic or eukaryotic. The organism can be unicellular or multicellular, A host organism is an organism comprising a host cell. In other embodiments, the host organism is photosynthetic. A photosynthetic organism is one that naturally photosynthesizes (e.g., an alga) or that is genetically engineered or otherwise modified to be photosynthetic. In some instances, a photosynthetic organism may be transformed with a construct or vector of the disclosure which renders all or part of the photosynthetic apparatus inoperable.

By way of example, a non-vascular photosynthetic microalga species (for example, C. reinhardtii, Nannochloropsis oceania, N. salina, D. salina, H. pluvalis, S. dimorphus, D. viridis, Chlorella sp., and D. tertiolecta) can be genetically engineered to produce a polypeptide of interest, for example a fibronectin domain 10 protein. Production of a fibronectin domain 10 protein in these microalgae can be achieved by engineering the microalgae to express the fibronectin domain 10 protein in the algal chloroplast or nucleus.

In other embodiments the host organism is a vascular plant. Non-limiting examples of such plants include various monocots and dicots, including high oil seed plants such as high oil seed Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carhamnus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, barley, oats, amaranth, potato, rice, tomato, and legumes (e.g., peas, beans, lentils, alfalfa, etc.).

The host cell can be prokaryotic. Examples of some prokaryotic organisms of the present disclosure include, but are not limited to, cyanobacteria (e.g., Synechococcus, Synechocystis, Athrospira, Gleocapsa, Oscillatoria, and, Pseudoanabaena). Suitable prokaryotic cells include, but are not limited to, any of a variety of laboratory strains of Escherichia coli, Lactobacillus sp., Salmonella sp., and Shigella sp. (for example, as described in Carrier et al. (1992) J. Immunol. 148:1176-1181; U.S. Pat. No. 6,447,784; and Sizemore et al. (1995) Science 270:299-302). Examples of Salmonella strains which can be employed in the present disclosure include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Pseudomonas pudita, Pseudomonas aeruginosa, Pseudomonas mevalonii, Rhodobacter sphaeroides, Rhodobacter capsulatus, Rhodospirillum rubrum, and Rhodococcus sp.

In some embodiments, the host organism is eukaryotic (e.g. green algae, red algae, brown algae). In some embodiments, the alga is a green algae, for example, a Chlorophycean. The algae can be unicellular or multicellular. Suitable eukaryotic host cells include, but are not limited to, yeast cells, insect cells, plant cells, fungal cells, and algal cells. Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharonmyces sp., Hansenula polymorpha, Kluyveromyces sp., Kluyveromyces lactis, Candida albicans, Aspergillus nidulans, Aspergillus niger, Aspergilius oryzae, Trichoderma reesei, Chrysosporium lucknowense, Fusarium sp., Fusarium gramineum, Fusarium venenaturn, Neurospora crassa, and Chlamydomonas reinhardtii. In other embodiments, the host cell is a microalga (e.g., Chlamydomonas reinhardtii, Dunalielia salina, Haematococcus pluvialis, Nannochloropsis oceania, N. salina, Scenedesmus dimorphus, Chlorella spp., D. viridis, or D. tertiolecta).

In some instances the organism is a rhodophyte, chlorophyte, heterokontophyte, tribophyte, glaucophyte, chlorarachniophyte, euglenoid, haptophyte, cryptomonad, dinoflagellum, or phytoplankton.

In some instances a host organism is vascular and photosynthetic. Examples of vascular plants include, but are not limited to, angiosperms, gymnosperms, rhyniophytes, or other tracheophytes.

In some instances a host organism is non-vascular and photosynthetic. As used herein, the term “non-vascular photosynthetic organism,” refers to any macroscopic or microscopic organism, including, but not limited to, algae, cyanobacteria and photosynthetic bacteria, which does not have a vascular system such as that found in vascular plants. Examples of non-vascular photosynthetic organisms include bryophtyes, such as marchantiophytes or anthocerotophytes. In some instances the organism is a cyanobacteria. In some instances, the organism is algae (e.g., macroalgae or microalgae). The algae can be unicellular or multicellular algae. For example, the microalgae Chlamydomonas reinhardtii may be transformed with a vector, or a linearized portion thereof, encoding one or more proteins of interest (e.g., VEGF or proinsulin).

Methods for algal transformation are described in U.S. Provisional Patent Application No. 60/142,091. The methods of the present disclosure can be carried out using algae, for example, the microalga, C. reinhardtii. The use of microalgae to express a polypeptide or protein complex according to a method of the disclosure provides the advantage that large populations of the microalgae can be grown, including commercially (Cyanotech Corp.; Kailua-Kona Hi.), thus allowing for production and, if desired, isolation of large amounts of a desired product.

The vectors of the present disclosure may be capable of stable or transient transformation of multiple photosynthetic organisms, including, but not limited to, photosynthetic bacteria (including cyanobacteria), cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptoraonads, dinophyta, dinoflagellata, pyrmnesiophyta, bacillariophyta, xanthophyta, eustigmatophyta, raphidophyta, phaeophyta, and phytoplankton. Other vectors of the present disclosure are capable of stable or transient transformation of, for example, C. reinhardtii, N. oceania, N. salina, D. salina, H. pluvalis, S. dimorphus, D. viridis, or D. tertiolecta.

Examples of appropriate hosts, include but are not limited to: bacterial cells, such as E. coli, Streptornyces, Salmonella typhimurium; fungal cells, such as yeast; insect cells, such as Drosophila S2 and Spodoptera Sf9; animal cells, such as CHO, COS or Bowes melanoma; adenoviruses; and plant cells. The selection of an appropriate host is deemed to be within the scope of those skilled in the art.

Polynucleotides selected and isolated as described herein are introduced into a suitable host cell. A suitable host cell is any cell which is capable of promoting recombination and/or reductive reassortment. The selected polynucleotides can be, for example, in a vector which includes appropriate control sequences. The host cell can be, for example, a higher eukaryotic cell, such as a mammalian cell, or a lower eukaryotic cell, such as a yeast cell, or the host cell can be a prokaryotic cell, such as a bacterial cell. Introduction of a construct (vector) into the host cell can be effected by, for example, calcium phosphate transfection, DEAE-Dextran mediated transfection, or electroporation.

Recombinant polypeptides, including therapeutic proteins, can be expressed in plants, allowing for the production of crops of such plants and, therefore, the ability to conveniently produce large amounts of a desired product, for example, a therapeutic protein. Accordingly, the methods of the disclosure can be practiced using any plant, including, for example, microalga and macroalgae, (such as marine algae and seaweeds), as well as plants that grow in soil.

In one embodiment, the host cell is a plant. The term “plant” is used broadly herein to refer to a eukaryotic organism containing plastids, such as chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, and roots. A part of a plant useful for propagation includes, for example, seeds, fruits, cuttings, seedlings, tubers, and rootstocks.

A method of the disclosure can generate a plant containing genomic DNA (for example, a nuclear and/or plastid genomic DNA) that is genetically modified to contain a stably integrated polynucleotide (for example, as described in Hager and Bock, Appl. Microbiol. Biotechnol. 54:302-310, 2000). Accordingly, the present disclosure further provides a transgenic plant, e.g. C. reinhardtii, which comprises one or more chloroplasts containing a polynucleotide encoding one or more exogenous polypeptides. A photosynthetic organism of the present disclosure comprises at least one host cell that is modified to generate, for example, a therapeutic protein.

Some of the host organisms useful in the disclosed embodiments are, for example, are extremophiles, such as hyperthermophiles, psychrophiles, psychrotrophs, halophiles, barophiles and acidophiles. Some of the host organisms which may be used to practice the present disclosure are halophilic (e.g., Dunaliella salina, D. viridis, or D. tertiolecta). For example, D. salina can grow in ocean water and salt lakes (for example, salinity from 30-300 parts per thousand) and high salinity media (e.g., artificial seawater medium, seawater nutrient agar, brackish water medium, and seawater medium). In some embodiments of the disclosure, a host cell expressing a protein of the present disclosure can be grown in a liquid environment which is, for example, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3 molar or higher concentrations of sodium chloride. One of skill in the art will recognize that other salts (sodium salts, calcium salts, potassium salts, or other salts) may also be present in the liquid environments.

Where a halophilic organism is utilized for the present disclosure, it may be transformed with any of the vectors described herein. For example, D. salina may be transformed with a vector which is capable of insertion into the chloroplast or nuclear genome and which contains nucleic acids which encode a protein (e.g., VEGF or proinsulin). Transformed halophilic organisms may then be grown in high-saline environments (e.g., salt lakes, salt ponds, and high-saline media) to produce the products (e.g., lipids) of interest. Isolation of the products may involve removing a transformed organism from a high-saline environment prior to extracting the product from the organism. In instances where the product is secreted into the surrounding environment, it may be necessary to desalinate the liquid environment prior to any further processing of the product.

The present disclosure further provides compositions comprising a genetically modified host cell. A composition comprises a genetically modified host cell; and will in some embodiments comprise one or more further components, which components are selected based in part on the intended use of the genetically modified host cell. Suitable components include, but are not limited to, salts; buffers; stabilizers; protease-inhibiting agents; cell membrane- and/or cell wall-preserving compounds, e.g., glycerol and dimethylsulfoxide; and nutritional media appropriate to the cell.

For the production of a protein, for example, a fibronectin domain protein, a host cell can be one that has been genetically modified to produce one or more fibronection domain proteins.

Culturing of Cells or Organisms

An organism may be grown under conditions which permit photosynthesis, however, this is not a requirement (e.g., a host organism may be grown in the absence of light). In some instances, the host organism may be genetically modified in such a way that its photosynthetic capability is diminished or destroyed. In growth conditions where a host organism is not capable of photosynthesis (e.g., because of the absence of light and/or genetic modification), typically, the organism will be provided with the necessary nutrients to support growth in the absence of photosynthesis. For example, a culture medium in (or on) which an organism is grown, may be supplemented with any required nutrient, including an organic carbon source, nitrogen source, phosphorous source, vitamins, metals, lipids, nucleic acids, micronutrients, and/or an organism-specific requirement. Organic carbon sources include any source of carbon which the host organism is able to metabolize including, but not limited to, acetate, simple carbohydrates (e.g., glucose, sucrose, and lactose), complex carbohydrates (e.g., starch and glycogen), proteins, and lipids. One of skill in the art will recognize that not all organisms will be able to sufficiently metabolize a particular nutrient and that nutrient mixtures may need to be modified from one organism to another in order to provide the appropriate nutrient mix.

Optimal growth of organisms occurs usually at a temperature of about 20° C. to about 25° C., although some organisms can still grow at a temperature of up to about 35° C. Active growth is typically performed in liquid culture. If the organisms are grown in a liquid medium and are shaken or mixed, the density of the cells can be anywhere from about 1 to 5×10⁸ cells/ml at the stationary phase. For example, the density of the cells at the stationary phase for Chlamydomonas sp. can be about 1 to 5×10⁷ cells/ml; the density of the cells at the stationary phase for Nannochloropsis sp. can be about 1 to 5×10⁸ cells/ml; the density of the cells at the stationary phase for Scenedesmus sp. can be about 1 to 5×10⁸ cells/ml; and the density of the cells at the stationary phase for Chlorella sp. can be about 1 to 5×10⁸ cells/ml. Exemplary cell densities at the stationary phase are as follows: Chlamydomonas sp. can be about 1×10⁷ cells/ml; Nannochloropsis sp. can be about 1×10⁸ cells/ml; Scenedesmus sp. can be about 1×10⁷ cells/ml; and Chlorella sp. can be about 1×10⁸ cells/ml. An exemplary growth rate may yield, for example, a two to four fold increase in cells per day, depending on the growth conditions. In addition, doubling times for organisms can be, for example, 5 hours to 30 hours. The organism can also be grown on solid media, for example, media containing about 1.5% agar, in plates or in slants.

One source of energy is fluorescent light that can be placed, for example, at a distance of about 1 inch to about two feet from the organism. Examples of types of fluorescent lights includes, for example, cool white and daylight. Bubbling with air or CO₂ improves the growth rate of the organism. Bubbling with CO₂ can be, for example, at 1% to 5% CO₂. If the lights are turned on and off at regular intervals (for example, 12:12 or 14:10 hours of light:dark) the cells of some organisms will become synchronized.

Long term storage of organisms can be achieved by streaking them onto plates, sealing the plates with, for example, Parafilm™, and placing them in dim light at about 10° C. to about 18° C. Alternatively, organisms may be grown as streaks or stabs into agar tubes, capped, and stored at about 10° C. to about 18° C. Both methods allow for the storage of the organisms for several months.

For longer storage, the organisms can be grown in liquid culture to mid to late log phase and then supplemented with a penetrating cryoprotective agent like DMSO or MeOH, and stored at less than −130° C. An exemplary range of DMSO concentrations that can be used is 5 to 8%. An exemplary range of MeOH concentrations that can be used is 3 to 9%.

Organisms can be grown on a defined minimal medium (for example, high salt medium (HSM), modified artificial sea water medium (MASM), or F/2 medium) with light as the sole energy source. In other instances, the organism can be grown in a medium (for example, tris acetate phosphate (TAP) medium), and supplemented with an organic carbon source.

Organisms, such as algae, can grow naturally in fresh water or marine water. Culture media for freshwater algae can be, for example, synthetic media, enriched media, soil water media, and solidified media, such as agar. Various culture media have been developed and used for the isolation and cultivation of fresh water algae and are described in Watanabe, M. W. (2005). Freshwater Culture Media. In R. A. Andersen (Ed.), Algal Culturing Techniques (pp. 13-20). Elsevier Academic Press. Culture media for marine algae can be, for example, artificial seawater media or natural seawater media. Guidelines for the preparation of media are described in Harrison, P. J. and Berges, J. A. (2005). Marine Culture Media. In R. A. Andersen (Ed.), Algal Culturing Techniques (pp. 21-33). Elsevier Academic Press.

Organisms may be grown in outdoor open water, such as ponds, the ocean, seas, rivers, waterbeds, marshes, shallow pools, lakes, aqueducts, and reservoirs. When grown in water, the organism can be contained in a halo-like object comprised of lego-like particles. The halo-like object encircles the organism and allows it to retain nutrients from the water beneath while keeping it in open sunlight.

In some instances, organisms can be grown in containers wherein each container comprises one or two organisms, or a plurality of organisms. The containers can be configured to float on water. For example, a container can be filled by a combination of air and water to make the container and the organism(s) in it buoyant. An organism that is adapted to grow in fresh water can thus be grown in salt water (i.e., the ocean) and vice versa. This mechanism allows for automatic death of the organism if there is any damage to the container.

Culturing techniques for algae are well know to one of skill in the art and are described, for example, in Freshwater Culture Media. In R. A. Andersen (Ed.), Algal Culturing Techniques. Elsevier Academic Press.

Because photosynthetic organisms, for example, algae, require sunlight, CO₂ and water for growth, they can be cultivated in, for example, open ponds and lakes. However, these open systems are more vulnerable to contamination than a closed system. One challenge with using an open system is that the organism of interest may not grow as quickly as a potential invader. This becomes a problem when another organism invades the liquid environment in which the organism of interest is growing, and the invading organism has a faster growth rate and takes over the system.

In addition, in open systems there is less control over water temperature, CO₂ concentration, and lighting conditions. The growing season of the organism is largely dependent on location and, aside from tropical areas, is limited to the warmer months of the year. In addition, in an open system, the number of different organisms that can be grown is limited to those that are able to survive in the chosen location. An open system, however, is cheaper to set up and/or maintain than a closed system.

Another approach to growing an organism is to use a semi-closed system, such as covering the pond or pool with a structure, for example, a “greenhouse-type” structure. While this can result in a smaller system, it addresses many of the problems associated with an open system. The advantages of a semi-closed system are that it can allow for a greater number of different organisms to be grown, it can allow for an organism to be dominant over an invading organism by allowing the organism of interest to out compete the invading organism for nutrients required for its growth, and it can extend the growing season for the organism. For example, if the system is heated, the organism can grow year round.

A variation of the pond system is an artificial pond, for example, a raceway pond. In these ponds, the organism, water, and nutrients circulate around a “racetrack.” Paddlewheels provide constant motion to the liquid in the racetrack, allowing for the organism to be circulated back to the surface of the liquid at a chosen frequency. Paddlewheels also provide a source of agitation and oxygenate the system. These raceway ponds can be enclosed, for example, in a building or a greenhouse, or can be located outdoors.

Raceway ponds are usually kept shallow because the organism needs to be exposed to sunlight, and sunlight can only penetrate the pond water to a limited depth. The depth of a raceway pond can be, for example, about 4 to about 12 inches. In addition, the volume of liquid that can be contained in a raceway pond can be, for example, about 200 liters to about 600,000 liters.

The raceway ponds can be operated in a continuous manner, with, for example, CO₂ and nutrients being constantly fed to the ponds, while water containing the organism is removed at the other end.

If the raceway pond is placed outdoors, there are several different ways to address the invasion of an unwanted organism. For example, the pH or salinity of the liquid in which the desired organism is in can be such that the invading organism either slows down its growth or dies.

Also, chemicals can be added to the liquid, such as bleach, or a pesticide can be added to the liquid, such as glyphosate. In addition, the organism of interest can be genetically modified such that it is better suited to survive in the liquid environment. Any one or more of the above strategies can be used to address the invasion of an unwanted organism.

Alternatively, organisms, such as algae, can be grown in closed structures such as photobioreactors, where the environment is under stricter control than in open systems or semi-closed systems. A photobioreactor is a bioreactor which incorporates some type of light source to provide photonic energy input into the reactor. The term photobioreactor can refer to a system closed to the environment and having no direct exchange of gases and contaminants with the environment. A photobioreactor can be described as an enclosed, illuminated culture vessel designed for controlled biomass production of phototrophic liquid cell suspension cultures. Examples of photobioreactors include, for example, glass containers, plastic tubes, tanks, plastic sleeves, and bags. Examples of light sources that can be used to provide the energy required to sustain photosynthesis include, for example, fluorescent bulbs, LEDs, and natural sunlight. Because these systems are closed everything that the organism needs to grow (for example, carbon dioxide, nutrients, water, and light) must be introduced into the bioreactor.

Photobioreactors, despite the costs to set up and maintain them, have several advantages over open systems, they can, for example, prevent or minimize contamination, permit axenic organism cultivation of monocultures (a culture consisting of only one species of organism), offer better control over the culture conditions (for example, pH, light, carbon dioxide, and temperature), prevent water evaporation, lower carbon dioxide losses due to out gassing, and permit higher cell concentrations.

On the other hand, certain requirements of photobioreactors, such as cooling, mixing, control of oxygen accumulation and biofouling, make these systems more expensive to build and operate than open systems or semi-closed systems.

Photobioreactors can be set up to be continually harvested (as is with the majority of the larger volume cultivation systems), or harvested one batch at a time (for example, as with polyethlyene bag cultivation) A batch photobioreactor is set up with, for example, nutrients, an organism (for example, algae), and water, and the organism is allowed to grow until the batch is harvested. A continuous photobioreactor can be harvested, for example, either continually, daily, or at fixed time intervals.

High density photobioreactors are described in, for example, Lee, et al., Biotech. Bioengineering 44:1161-1167, 1994. Other types of bioreactors, such as those for sewage and waste water treatments, are described in, Sawayama, et al., Appl. Micro. Biotech., 41:729-731, 1994. Additional examples of photobioreactors are described in, U.S. Appl. Publ. No. 2005/0260553, U.S. Pat. No. 5,958,761, and U.S. Pat. No. 6,083,740. Also, organisms, such as algae may be mass-cultured for the removal of heavy metals (for example, as described in Wilkinson, Biotech. Letters, 11:861-864, 1989), hydrogen (for example, as described in U.S. Patent Application Publication No. 2003/0162273), and pharmaceutical compounds from a water, soil, or other source or sample. Organisms can also be cultured in conventional fermentation bioreactors, which include, but are not limited to, batch, fed-batch, cell recycle, and continuous fermentors. Additional methods of culturing organisms and variations of the methods described herein are known to one of skill in the art.

Organisms can also be grown near ethanol production plants or other facilities or regions (e.g., cities and highways) generating CO₂. As such, the methods herein contemplate business methods for selling carbon credits to ethanol plants or other facilities or regions generating CO₂ while making fuels or fuel products by growing one or more of the organisms described herein near the ethanol production plant, facility, or region.

The organism of interest, grown in any of the systems described herein, can be, for example, continually harvested, or harvested one batch at a time.

CO₂ can be delivered to any of the systems described herein, for example, by bubbling in CO₂ from under the surface of the liquid containing the organism. Also, sparges can be used to inject CO₂ into the liquid. Spargers are, for example, porous disc or tube assemblies that are also referred to as Bubblers, Carbonators, Aerators, Porous Stones and Diffusers.

Nutrients that can be used in the systems described herein include, for example, nitrogen (in the form of NO₃⁻ or NH₄⁺), phosphorus, and trace metals (Fe, Mg, K, Ca, Co, Cu, Mn, Mo, Zn, V, and B). The nutrients can come, for example, in a solid form or in a liquid form. If the nutrients are in a solid form they can be mixed with, for example, fresh or salt water prior to being delivered to the liquid containing the organism, or prior to being delivered to a photobioreactor.

Organisms can be grown in cultures, for example large scale cultures, where large scale cultures refers to growth of cultures in volumes of greater than about 6 liters, or greater than about 10 liters, or greater than about 20 liters. Large scale growth can also be growth of cultures in volumes of 50 liters or more, 100 liters or more, or 200 liters or more. Large scale growth can be growth of cultures in, for example, ponds, containers, vessels, or other areas, where the pond, container, vessel, or area that contains the culture is for example, at lease 5 square meters, at least 10 square meters, at least 200 square meters, at least 500 square meters, at least 1,500 square meters, at least 2,500 square meters, in area, or greater.

Chlamydomonas sp., Nannochloropsis sp., Scenedesmus sp., and Chlorella sp. are exemplary algae that can be cultured as described herein and can grow under a wide array of conditions.

One organism that can be cultured as described herein is a commonly used laboratory species C. reinhardtii. Cells of this species are haploid, and can grow on a simple medium of inorganic salts, using photosynthesis to provide energy. This organism can also grow in total darkness if acetate is provided as a carbon source. C. reinhardtii can be readily grown at room temperature under standard fluorescent lights. In addition, the cells can be synchronized by placing them on a light-dark cycle. Other methods of culturing C. reinhardtii cells are known to one of skill in the art.

Polynucleotides and Polypeptides

Also provided are isolated polynucleotides encoding a protein, for example, a high-mobility group box 1 protein, described herein. As used herein “isolated polynucleotide” means a polynucleotide that is free of one or both of the nucleotide sequences which flank the polynucleotide in the naturally-occurring genome of the organism from which the polynucleotide is derived. The term includes, for example, a polynucleotide or fragment thereof that is incorporated into a vector or expression cassette; into an autonomously replicating plasmid or virus; into the genomic DNA of a prokaryote or eukaryote; or that exists as a separate molecule independent of other polynucleotides. It also includes a recombinant polynucleotide that is part of a hybrid polynucleotide, for example, one encoding a polypeptide sequence.

The novel proteins of the present disclosure can be made by any method known in the art. The protein may be synthesized using either solid-phase peptide synthesis or by classical solution peptide synthesis also known as liquid-phase peptide synthesis. Using Val-Pro-Pro, Enalapril and Lisinopril as starting templates, several series of peptide analogs such as X-Pro-Pro, X-Ala-Pro, and X-Lys-Pro, wherein X represents any amino acid residue, may be synthesized using solid-phase or liquid-phase peptide synthesis. Methods for carrying out liquid phase synthesis of libraries of peptides and oligonucleotides coupled to a soluble oligomeric support have also been described. Bayer, Ernst and Mutter, Manfred, Nature 237:512-513 (1972); Bayer, Ernst, et al., J. Am. Chem. Soc. 96:7333-7336 (1974); Bonora, Gian Maria, et al., Nucleic Acids Res. 18:3155-3159 (1990). Liquid phase synthetic methods have the advantage over solid phase synthetic methods in that liquid phase synthesis methods do not require a structure present on a first reactant which is suitable for attaching the reactant to the solid phase. Also, liquid phase synthesis methods do not require avoiding chemical conditions which may cleave the bond between the solid phase and the first reactant (or intermediate product). In addition, reactions in a homogeneous solution may give better yields and more complete reactions than those obtained in heterogeneous solid phase/liquid phase systems such as those present in solid phase synthesis.

In oligomer-supported liquid phase synthesis the growing product is attached to a large soluble polymeric group. The product from each step of the synthesis can then be separated from unreacted reactants based on the large difference in size between the relatively large polymer-attached product and the unreacted reactants. This permits reactions to take place in homogeneous solutions, and eliminates tedious purification steps associated with traditional liquid phase synthesis. Oligomer-supported liquid phase synthesis has also been adapted to automatic liquid phase synthesis of peptides. Bayer, Ernst, et al., Peptides: Chemistry, Structure, Biology, 426-432.

For solid-phase peptide synthesis, the procedure entails the sequential assembly of the appropriate amino acids into a peptide of a desired sequence while the end of the growing peptide is linked to an insoluble support. Usually, the carboxyl terminus of the peptide is linked to a polymer from which it can be liberated upon treatment with a cleavage reagent. In a common method, an amino acid is bound to a resin particle, and the peptide generated in a stepwise manner by successive additions of protected amino acids to produce a chain of amino acids. Modifications of the technique described by Merrifield are commonly used. See, e.g., Merrifield, J. Am. Chem. Soc. 96: 2989-93 (1964). In an automated solid-phase method, peptides are synthesized by loading the carboxy-terminal amino acid onto an organic linker (e.g., PAM, 4-oxymethylphenylacetamidomethyl), which is covalently attached to an insoluble polystyrene resin cross-linked with divinyl benzene. The terminal amine may be protected by blocking with t-butyloxycarbonyl. Hydroxyl- and carboxyl-groups are commonly protected by blocking with O-benzyl groups. Synthesis is accomplished in an automated peptide synthesizer, such as that available from Applied Biosystems (Foster City, Calif.). Following synthesis, the product may be removed from the resin. The blocking groups are removed by using hydrofluoric acid or trifluoromethyl sulfonic acid according to established methods. A routine synthesis may produce 0.5 mmole of peptide resin. Following cleavage and purification, a yield of approximately 60 to 70% is typically produced. Purification of the product peptides is accomplished by, for example, crystallizing the peptide from an organic solvent such as methyl-butyl ether, then dissolving in distilled water, and using dialysis (if the molecular weight of the subject peptide is greater than about 500 daltons) or reverse high pressure liquid chromatography (e.g., using a C¹⁸ column with 0.1% trifluoroacetic acid and acetonitrile as solvents) if the molecular weight of the peptide is less than 500 daltons. Purified peptide may be lyophilized and stored in a dry state until use. Analysis of the resulting peptides may be accomplished using the common methods of analytical high pressure liquid chromatography (HPLC) and electrospray mass spectrometry (ES-MS).

In other cases, a therapeutic protein, for example, a fibronectin domain protein, is produced by recombinant methods. For production of any of the proteins described herein, host cells transformed with an expression vector containing the polynucleotide encoding such a protein can be used. The host cell can be a higher eukaryotic cell, such as a mammalian cell, or a lower eukaryotic cell such as a yeast or algal cell, or the host can be a prokaryotic cell such as a bacterial cell. Introduction of the expression vector into the host cell can be accomplished by a variety of methods including calcium phosphate transfection, DEAE-dextran mediated transfection, polybrene, protoplast fusion, liposomes, direct microinjection into the nuclei, scrape loading, biolistic transformation and electroporation. Large scale production of proteins from recombinant organisms is a well established process practiced on a commercial scale and well within the capabilities of one skilled in the art.

It should be recognized that the present disclosure is not limited to transgenic cells, organisms, and plastids containing a protein or proteins as disclosed herein, but also encompasses such cells, organisms, and plastids transformed with additional nucleotide sequences. These additional sequences may be contained in a single vector either operatively linked to a single promoter or linked to multiple promoters, e.g. one promoter for each sequence. Alternatively, the additional coding sequences may be contained in a plurality of additional vectors. When a plurality of vectors are used., they can be introduced into the host cell or organism simultaneously or sequentially.

Additional embodiments provide a plastid, and in particular a chloroplast, transformed with a polynucleotide encoding a protein of the present disclosure. The protein may be introduced into the genome of the plastid using any of the methods described herein or otherwvise known in the art. The plastid may be contained in the organism in which it naturally occurs. Alternatively, the plastid may be an isolated plastid, that is, a plastid that has been removed from the cell in which it normally occurs. Methods for the isolation of plastids are known in the art and can be found, for example, in Maliga et al., Methods in Plant Molecular Biology, Cold Spring Harbor Laboratory Press, 1995; Gupta and Singh, J. Biosci., 21:819 (1996); and Camara et al., Plant Physiol., 73:94 (1983). The isolated plastid transformed with a protein of the present disclosure can be introduced into a host cell. The host cell can be one that naturally contains the plastid or one in which the plastid is not naturally found.

Also within the scope of the present disclosure are artificial plastid genomes, for example chloroplast genomes, that contain nucleotide sequences encoding any one or more of the proteins of the present disclosure. Methods for the assembly of artificial plastid genomes can be found in co-pending U.S. patent application Ser. No. 12/287,230 filed Oct. 6, 2008, published as U.S. Publication No. 2009/0123977 on May 14, 2009, and U.S. patent application Ser. No. 12/384,893 filed Apr. 8, 2009, published as U.S. Publication No. 2009/0269816 on Oct. 29, 2009, each of which is incorporated by reference in its entirety.

Introduction of Polynucleotide into a Host Organism or Cell

To generate a genetically modified host cell, a polynucleotide, or a polynucleotide cloned into a vector, is introduced stably or transiently into a host cell, using established techniques, including, but not limited to, electroporation, calcium phosphate precipitation, DEAE-dextran mediated transfection, and liposome-mediated transfection. For transformation, a polynucleotide of the present disclosure will generally further include a selectable marker, e.g., any of several well-known selectable markers such as neomycin resistance, ampicillin resistance, tetracycline resistance, chloramphenicol resistance, and kanamycin resistance.

A polynucleotide or recombinant nucleic acid molecule described herein, can be introduced into a cell (e.g., alga cell) using any method known in the art. A polynucleotide can be introduced into a cell by a variety of methods, which are well known in the art and selected, in part, based on the particular host cell. For example, the polynucleotide can be introduced into a cell using a direct gene transfer method such as electroporation or microprojectile mediated (biolistic) transformation using a particle gun, or the “glass bead method,” or by pollen-mediated transformation, liposome-mediated transformation, transformation using wounded or enzyme-degraded immature embryos, or wounded or enzyme-degraded embryogenic callus (for example, as described in Potrykus, Ann. Rev. Plant. Physiol. Plant Mol. Biol. 42:205-225, 1991).

As discussed above, microprojectile mediated transformation can be used to introduce a polynucleotide into a cell (for example, as described in Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed, into a cell using a device such as the BIOLISTIC PD-1000 particle gun (BioRad; Hercules Calif.). Methods for the transformation using biolistic methods are well known in the art (for example, as described in Christou, Trends in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (for example, as described in Duan et al., Nature Biotech. 14:494-498, 1996; and Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, and the glass bead agitation method.

The basic techniques used for transformation and expression in photosynthetic microorganisms are similar to those commonly used for E. coli, Saccharomyces cerevisiae and other species. Transformation methods customized for a photosynthetic microorganisms, e.g., the chloroplast of a strain of algae, are known in the art. These methods have been described in a number of texts for standard molecular biological manipulation (see Packer & Glaser, 1988, “Cyanobacteria”, Meth. Enzymol., Vol. 167; Weissbach & Weissbach, 1988, “Methods for plant molecular biology,” Academic Press, New York, Sambrook, Fritsch & Maniatis, 1989, “Molecular Cloning: A laboratory manual,” 2nd edition Cold Spring Harbor Laboratory Press, Cold Spring Harbor, N.Y.; and Clark M S, 1997, Plant Molecular Biology, Springer, N.Y.). These methods include, for example, biolistic devices (See, for example, Sanford, Trends In Biotech. (1988) δ: 299-302, U.S. Pat. No. 4,945,050; electroporation (Fromm et al., Proc. Nat'l. Acad. Sci. (USA) (1985) 82: 5824-5828); use of a laser beam, electroporation, microinjection or any other method capable of introducing DNA into a host cell.

Plastid transformation is a routine and well known method for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome. In some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation (Svab et al., Proc. Natl. Acad. Sci., USA 87:8526-8530, 1990), and can result in stable homoplasmic transformants, at a frequency of approximately one per 100 bombardments of target leaves.

A further refinement in chloroplast transformation/expression technology that facilitates control over the timing and tissue pattern of expression of introduced DNA coding sequences in plant plastid genomes has been described in PCT International Publication WO 95/16783 and U.S. Pat. No. 5,576,198. This method involves the introduction into plant cells of constructs for nuclear transformation that provide for the expression of a viral single subunit RNA polymerase and targeting of this polymerase into the plastids via fusion to a plastid transit peptide. Transformation of plastids with DNA constructs comprising a viral single subunit RNA polymerase-specific promoter specific to the RNA polymerase expressed from the nuclear expression constructs operably linked to DNA coding sequences of interest permits control of the plastid expression constructs in a tissue and/or developmental specific manner in plants comprising both the nuclear polymerase construct and the plastid expression constructs. Expression of the nuclear RNA polymerase coding sequence can be placed under the control of either a constitutive promoter, or a tissue- or developmental stage-specific promoter, thereby extending this control to the plastid expression construct responsive to the plastid-targeted, nuclear-encoded viral RNA polymerase.

When nuclear transformation is utilized, the protein can be modified for plastid targeting by employing plant cell nuclear transformation constructs wherein DNA coding sequences of interest are fused to any of the available transit peptide sequences capable of facilitating transport of the encoded proteins into plant plastids, and driving expression by employing an appropriate promoter. Targeting of the protein can be achieved by fusing DNA encoding plastid, e.g., chloroplast, leucoplast, amyloplast, etc., transit peptide sequences to the 5′ end of the DNA encoding the protein. The sequences that encode a transit peptide region can be obtained, for example, from plant nuclear-encoded plastid proteins, such as the small subunit (SSU) of ribulose bisphosphate carboxylase, EPSP synthase, plant fatty acid biosynthesis related genes including fatty acyl-ACP thioesterases, acyl carrier protein (ACP), stearoyl-ACP desaturase, β-ketoacyl-ACP synthase and acyl-ACP thioesterase, or LHCPII genes, etc. Plastid transit peptide sequences can also be obtained from nucleic acid sequences encoding carotenoid biosynthetic enzymes, such as GGPP synthase, phytoene synthase, and phytoene desaturase. Other transit peptide sequences are disclosed in Von Heijne et al., (1991) Plant Mol. Biol. Rep. 9: 104; Clark et al. (1989) J. Biol. Chem. 264: 17544; della-Cioppa et al. (1987) Plant Physiol. 84: 965; Romer et al. (1993) Biochem. Biophys. Res. Commun. 196: 1414; and Shah et al. (1986) Science 233: 478. Another transit peptide sequence is that of the intact ACCase from Chlamydomonas (genbank EDO96563, amino acids 1-33). The encoding sequence for a transit peptide effective in transport to plastids can include all or a portion of the encoding sequence for a particular transit peptide, and may also contain portions of the mature protein encoding sequence associated with a particular transit peptide. Numerous examples of transit peptides that can be used to deliver target proteins into plastids exist, and the particular transit peptide encoding sequences useful in the present disclosure are not critical as long as delivery into a plastid is obtained. Proteolytic processing within the plastid then produces the mature protein. This technique has proven successful with enzymes involved in polyhydroxyalkanoate biosynthesis (Nawrath et al. (1994) Proc. Natl. Acad. Sci. USA 91: 12760), and neomycin phosphotransferase II (NPT-II) and CP4 EPSPS (Padgette et al. (1995) Crop Sci. 35: 1451), for example.

Of interest are transit peptide sequences derived from enzymes known to be imported into the leucoplasts of seeds. Examples of enzymes containing useful transit peptides include those related to lipid biosynthesis (e.g., subunits of the plastid-targeted dicot acetyl-CoA carboxylase, biotin carboxylase, biotin carboxyl carrier protein, a-carboxy-transferase, and plastid-targeted monocot multifunctional acetyl-CoA carboxylase (Mw, 220,000); plastidic subunits of the fatty acid synthase complex (e.g., acyl carrier protein (ACP), malonyl-ACP synthase, KASI, KASII, and KASIII); steroyl-ACP desaturase; thioesterases (specific for short, medium, and long chain acyl ACP); plastid-targeted acyl transferases (e.g., glycerol-3-phosphate and acyl transferase); enzymes involved in the biosynthesis of aspartate family amino acids; phytoene synthase; gibberellic acid biosynthesis (e.g., ent-kaurene synthases 1 and 2); and carotenoid biosynthesis (e.g., lycopene synthase).

In some embodiments, an alga is transformed with a nucleic acid which encodes a therapeutic protein of interest, for example, 10FN3, 14FN3, proinsulin, VEGF, or HMGB1.

In one embodiment, a transformation may introduce a nucleic acid into a plastid of the host alga (e.g., chloroplast). In another embodiment, a transformation may introduce a nucleic acid into the nuclear genome of the host alga. In still another embodiment, a transformation may introduce nucleic acids into both the nuclear genome and into a plastid.

Transformed cells can be plated on selective media following introduction of exogenous nucleic acids. This method may also comprise several steps for screening. A screen of primary transformants can be conducted to determine which clones have proper insertion of the exogenous nucleic acids. Clones which show the proper integration may be propagated and re-screened to ensure genetic stability. Such methodology ensures that the transformants contain the genes of interest. In many instances, such screening is performed by polymerase chain reaction (PCR); however, any other appropriate technique known in the art may be utilized. Many different methods of PCR are known in the art (e.g., nested PCR, real time PCR). For any given screen, one of skill in the art will recognize that PCR components may be varied to achieve optimal screening results. For example, magnesium concentration may need to be adjusted upwards when PCR is performed on disrupted alga cells to which (which chelates magnesium) is added to chelate toxic metals. Following the screening for clones with the proper integration of exogenous nucleic acids, clones can be screened for the presence of the encoded protein(s) and/or products. Protein expression screening can be performed by Western blot analysis and/or enzyme activity assays. Transporter and/or product screening may be performed by any method known in the art, for example ATP turnover assay, substrate transport assay, HPLC or gas chromatography.

The expression of the therapeutic protein can be accomplished by inserting a polynucleotide sequence (gene) encoding the protein or enzyme into the chloroplast or nuclear genome of a microalgae. The modified strain of microalgae can be made homoplasmic to ensure that the polynucleotide will be stably maintained in the chloroplast genome of all descendents. A microalga is homoplasmic for a gene when the inserted gene is present in all copies of the chloroplast genome, for example. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% or more of the total soluble plant protein. The process of determining the plasmic state of an organism of the present disclosure involves screening transformants for the presence of exogenous nucleic acids and the absence of wild-type nucleic acids at a given locus of interest.

Vectors

Construct, vector and plasmid are used interchangeably throughout the disclosure. Nucleic acids encoding the proteins described herein can be contained in vectors, including cloning and expression vectors. A cloning vector is a self-replicating DNA molecule that serves to transfer a DNA segment into a host cell. Three common types of cloning vectors are bacterial plasmids, phages, and other viruses. An expression vector is a cloning vector designed so that a coding sequence inserted at a particular site will be transcribed and translated into a protein. Both cloning and expression vectors can contain nucleotide sequences that allow the vectors to replicate in one or more suitable host cells. In cloning vectors, this sequence is generally one that enables the vector to replicate independently of the host cell chromosomes, and also includes either origins of replication or autonomously replicating sequences.

In some embodiments, a polynucleotide of the present disclosure is cloned or inserted into an expression vector using cloning techniques know to one of skill in the art. The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2nd Ed., Cold Spring Harbor Press, (1989) and Ausubel et al., Short Protocols in Molecular Biology, 2nd Ed., John Wiley & Sons (1992).

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, and herpes simplex virus), PI-based artificial chromosomes, yeast plasmids, yeast artificial chromosomes, and any other vectors specific for specific hosts of interest (such as E. coli and yeast). Thus, for example, a polynucleotide encoding VEGF can be inserted into any one of a variety of expression vectors that are capable of expressing the protein. Such vectors can include, for example, chromosomal, nonchromosomal and synthetic DNA sequences.

Suitable expression vectors include chromosomal, non-chromosomal and synthetic DNA sequences, for example, SV 40 derivatives; bacterial plasmids; phage DNA; baculovirus; yeast plasmids; vectors derived from combinations of plasmids and phage DNA; and viral DNA such as vaccinia, adenovirus, fowl pox virus, and pseudorabies. In addition, any other vector that is replicable and viable in the host may be used. For example, vectors such as Ble2A, Arg7/2A, and SEnuc357 can be used for the expression of a protein.

Numerous suitable expression vectors are known to those of skill in the art. The following vectors are provided by way of example; for bacterial host cells: pQE vectors (Qiagen), pBluescript plasmids, pNH vectors, lambda-ZAP vectors (Stratagene), pTrc99a, pKK223-3, pDR540, and pRIT2T (Pharmacia); for eukaryotic host cells: pXT1, pSG5 (Stratagene), pSVK3, pBPV, pMSG, pET21a-d(+) vectors (Novagen), and pSVLSV40 (Pharmacia). However, any other plasmid or other vector may be used so long as it is compatible with the host cell.

The expression vector, or a linearized portion thereof, can comprise one or more exogenous nucleotide sequences. Examples of exogenous nucleotide sequences that can be transformed into a host include nucleic acid sequences that code for mammalian genes, such as human genes. Example of human genes useful in the disclosed embodiments are growth factors, such as VEGF, or insulin. In some instances, an exogenous sequence is flanked by two sequences that have homology to sequences contained in the host organism to be transformed.

Homologous sequences are, for example, those that have at least 50%, at least 60%, at least 70%, at least 80%, at least 90%, at least 95%, at least 98%, or at least 99% sequence identity to a reference amino acid sequence or nucleotide sequence, for example, the amino acid sequence or nucleotide sequence that is found in the host cell from which the protein is naturally obtained from or derived from.

A nucleotide sequence can also be homologous to a codon-optimized gene sequence. For example, a nucleotide sequence can have, for example, at least 50%, at least 60%, at least 70%, at least 80%, at least 90%, at least 95%, at least 98%, or at least 99% nucleic acid sequence identity to the codon-optimized gene sequence.

An exogenous nucleotide sequence comprising a nucleic acid encoding a therapeutic protein can be flanked by two homologous sequences, one on each side. The first and second homologous sequences enable recombination of the exogenous sequence into the genome of the host organism to be transformed. The first and second homologous sequences can be at least 100, at least 200, at least 300, at least 400, at least 500, or at least 1500 nucleotides in length.

In some embodiments, about 0.5 to about 1.5 kb flanking nucleotide sequences of chloroplast genomic DNA may be used. In other embodiments about 0.5 to about 1.5 kb flanking nucleotide sequences of nuclear genomic DNA may be used, or about 2.0 to about 5.0 kb may be used.

In some embodiments, the vector may comprise nucleotide sequences that are codon-biased for expression in the organism being transformed. In another embodiment, a gene of interest, for example, a therapeutic gene, may comprise nucleotide sequences that are codon-biased for expression in the organism being transformed. In addition, the nucleotide sequence of a tag may be codon-biased or codon-optimized for expression in the organism being transformed.

A polynucleotide sequence may comprise nucleotide sequences that are codon biased for expression in the organism being transformed. The skilled artisan is well aware of the “codon-bias” exhibited by a specific host cell in usage of nucleotide codons to specify a given amino acid. Without being bound by theory, by using a host cell's preferred codons, the rate of translation may be greater. Therefore, when synthesizing a gene for improved expression in a host cell, it may be desirable to design the gene such that its frequency of codon usage approaches the frequency of preferred codon usage of the host cell. In some organisms, codon bias differs between the nuclear genome and organelle genomes, thus, codon optimization or biasing may be performed for the target genome (e.g., nuclear codon biased or chloroplast codon biased). In some embodiments, codon biasing occurs before mutagenesis to generate a polypeptide. In other embodiments, codon biasing occurs after mutagenesis to generate a polynucleotide. In yet other embodiments, codon biasing occurs before mutagenesis as well as after mutagenesis. Codon bias is described in detail herein.

In some embodiments, a vector comprises a polynucleotide operably linked to one or more control elements, such as a promoter and/or a transcription terminator. A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading phase. Linking is achieved by ligation at restriction enzyme sites. If suitable restriction sites are not available, then synthetic oligonucleotide adapters or linkers can be used as is known to those skilled in the art. Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

A vector in some embodiments provides for amplification of the copy number of a polynucleotide. A vector can be, for example, an expression vector that provides for expression of a therapeutic protein in a host cell, e.g., a prokaryotic host cell or a eukaryotic host cell.

A polynucleotide or polynucleotides can be contained in a vector or vectors. For example, where a second (or more) nucleic acid molecule is desired, the second nucleic acid molecule can be contained in a vector, which can, but need not be, the same vector as that containing the first nucleic acid molecule. The vector can be any vector useful for introducing a polynucleotide into a genome and can include a nucleotide sequence of genomic DNA (e.g., nuclear or plastid) that is sufficient to undergo homologous recombination with genomic DNA, for example, a nucleotide sequence comprising about 400 to about 1500 or more substantially contiguous nucleotides of genomic DNA.

A regulatory or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, a hairpin structure, an RNAase stability element, an initiation (start) codon, a splicing signal for intron excision and maintenance of a correct reading frame, a STOP codon, an amber or ochre codon, and an IRES. A regulatory element can include a promoter and transcriptional and translational stop signals. Elements may be provided with linkers for the purpose of introducing specific restriction sites facilitating ligation of the control sequences with the coding region of a nucleotide sequence encoding a polypeptide. Additionally, a sequence comprising a cell compartmentalization signal (i.e., a sequence that targets a polypeptide to the cytosol, nucleus, chloroplast membrane or cell membrane) can be attached to the polynucleotide encoding a protein of interest. Such signals are well known in the art and have been widely reported (see, e.g., U.S. Pat. No. 5,776,689).

In a vector, a nucleotide sequence of interest is operably linked to a promoter recognized by the host cell to direct mRNA synthesis. Promoters are untranslated sequences located generally 100 to 1000 base pairs (bp) upstream from the start codon of a structural gene that regulate the transcription and translation of nucleic acid sequences under their control.

Promoters useful for the present disclosure may come from any source (e.g., viral, bacterial, fungal, protist, and animal). The promoters contemplated herein can be specific to photosynthetic organisms, non-vascular photosynthetic organisms, and vascular photosynthetic organisms (e.g., algae, flowering plants). In some instances, the nucleic acids above are inserted into a vector that comprises a promoter of a photosynthetic organism, e.g., algae. The promoter can be a constitutive promoter or an inducible promoter. A promoter typically includes necessary nucleic acid sequences near the start site of transcription, (e.g., a TATA element). Common promoters used in expression vectors include, but are not limited to, LTR or SV40 promoter, the E. coli lac or trp promoters, and the phage lambda PL promoter. Non-limiting examples of promoters are endogenous promoters such as the psbA and atpA promoter. Other promoters known to control the expression of genes in prokaryotic or eukaryotic cells can be used and are known to those skilled in the art. Expression vectors may also contain a ribosome binding site for translation initiation, and a transcription terminator. The vector may also contain sequences useful for the amplification of gene expression.

A “constitutive” promoter is, for example, a promoter that is active under most environmental and developmental conditions. Constitutive promoters can, for example, maintain a relatively constant level of transcription.

An “inducible” promoter is a promoter that is active under controllable environmental or developmental conditions. For example, inducible promoters are promoters that initiate increased levels of transcription from DNA under their control in response to some change in the environment, e.g. the presence or absence of a nutrient or a change in temperature.

Examples of inducible promoters/regulatory elements include, for example, a nitrate-inducible promoter (for example, as described in Bock et al., Plant Mol. Bio. 17:9 (1991)), or a light-inducible promoter, (for example, as described in Feinbaum et al., Mol. Gen. Genet. 226:449 (1991); and Lam and Chua, Science 248:471 (1990)), or a heat responsive promoter (for example, as described in Muller et al., Gene 111: 165-73 (1992)).

In some embodiments, a polynucleotide of the present disclosure includes a nucleotide sequence encoding a therapeutic protein of the present disclosure, where the nucleotide sequence encoding the polypeptide is operably linked to an inducible promoter. Inducible promoters are well known in the art. Suitable inducible promoters include, but are not limited to, the pL of bacteriophage λ; Placo; Ptrp; Ptac (Ptrp-lac hybrid promoter); an isopropyl-beta-D-thiogalactopyranoside (IPTG)-inducible promoter, e.g., a lacZ promoter; a tetracycline-inducible promoter; an arabinose inducible promoter, e.g., P_{BAD} (for example, as described in Guzman et al. (1995) J. Bacteriol. 177:4121-4130); a xylose-inducible promoter, e.g., Pxyl (for example, as described in Kim et al. (1996) Gene 181:71-76); a GAL1 promoter; a tryptophan promoter; a lac promoter; an alcohol-inducible promoter, e.g., a methanol-inducible promoter, an ethanol-inducible promoter; a raffinose-inducible promoter; and a heat-inducible promoter, e.g., heat inducible lambda P_{L} promoter and a promoter controlled by a heat-sensitive repressor (e.g., C1857-repressed lambda-based expression vectors; for example, as described in Hoffmann et al. (1999) FEMS Microbiol Lett. 177(2):327-34).

In some embodiments, a polynucleotide of the present disclosure includes a nucleotide sequence encoding a therapeutic protein of the present disclosure, where the nucleotide sequence encoding the polypeptide is operably linked to a constitutive promoter. Suitable constitutive promoters for use in prokaryotic cells are known in the art and include, but are not limited to, a sigma70 promoter, and a consensus sigma70 promoter.

Suitable promoters for use in prokaryotic host cells include, but are not limited to, a bacteriophage T7 RNA polymerase promoter; a trip promoter; a lac operon promoter; a hybrid promoter, e.g., a lac/tac hybrid promoter, a tac/tre hybrid promoter, a trp/lac promoter, a T7/lac promoter; a trc promoter; a tac promoter; an araBAD promoter; in vivo regulated promoters, such as an ssaG promoter or a related promoter (for example, as described in U.S. Patent Publication No. 20040131637), a pagC promoter (for example, as described in Pulkkinen and Miller, J. Bacteriol., 1991: 173(1): 86-93; and Alpuche-Aranda et al., PNAS, 1992; 89(21): 10079-83), a nirB promoter (for example, as described in Harborne et al. (1992) Mol. Micro. 6:2805-2813; Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (for example, GenBank Accession Nos. AX798980, AX798961, and AX798183); a stationary phase promoter, e.g., a dps promoter, an spy promoter; a promoter derived from the pathogenicity island SPI-2 (for example, as described in WO96/17951); an actA promoter (for example, as described in Shetron-Rama et al. (2002) infect. Immun. 70:1087-1096); an rpsM promoter (for example, as described in Valdivia and Falkow (1996). Mol. Microbiol. 22:367-378); a tet promoter (for example, as described in Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); and an SP6 promoter (for example, as described in Melton et al. (1984) Nucl. Acids Res. 12:7035-7056).

In yeast, a number of vectors containing constitutive or inducible promoters may be used. For a review of such vectors see, Current Protocols in Molecular Biology, Vol. 2, 1988, Ed. Ausubel, et al., Greene Publish. Assoc. & Wiley Interscience, Ch. 13; Grant, et al., 1987, Expression and Secretion Vectors for Yeast, in Methods in Enzymology, Eds. Wu & Grossman, 31987, Acad. Press, N.Y., Vol. 153, pp. 516-544; Glover, 1986, DNA Cloning, Vol. II, IRL Press, Wash., D.C., Ch. 3; Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (for example, as described in Cloning in Yeast, Ch. 3, R. Rothstein In: DNA Cloning Vol. 11, A Practical Approach, Ed. DM Glover, 1986, IRL Press, Wash., D.C.). Alternatively, vectors may be used which promote integration of foreign DNA sequences into the yeast chromosome.

Non-limiting examples of suitable eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

A vector utilized in the practice of the disclosure also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, and sequences that encode a selectable marker. As such, the vector can contain, for example, one or more cloning sites such as a multiple cloning site, which can, but need not, be positioned such that an exogenous polynucleotide can be inserted into the vector and operatively linked to a desired element.

The vector also can contain a prokaryote origin of replication (ori), for example, an E. coli ori or a cosmid ori, thus allowing passage of the vector into a prokaryote host cell, as well as into a plant chloroplast. Various bacterial and viral origins of replication are well known to those skilled in the art and include, but are not limited to the pBR322 plasmid origin, the 2u plasmid origin, and the SV40, polyoma, adenovirus, VSV, and BPV viral origins.

A regulatory or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, an IRES. Additionally, an element can be a cell compartmentalization signal (i.e., a sequence that targets a polypeptide to the cytosol, nucleus, chloroplast membrane or cell membrane). In some aspects of the present disclosure, a cell compartmentalization signal (e.g., a cell membrane targeting sequence) may be ligated to a gene and/or transcript, such that translation of the gene occurs in the chloroplast. In other aspects, a cell compartmentalization signal may be ligated to a gene such that, following translation of the gene, the protein is transported to the cell membrane. Cell compartmentalization signals are well known in the art and have been widely reported (see, e.g., U.S. Pat. No. 5,776,689).

A vector, or a linearized portion thereof, may include a nucleotide sequence encoding a reporter polypeptide or other selectable marker. The term “reporter” or “selectable marker” refers to a polynucleotide (or encoded polypeptide) that confers a detectable phenotype.

A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation (for example, as described in Giacomin, Plant Sci. 116:59-72, 1996; Scikantha, J. Bacteriol. 178:121, 1996; Gerdes, FEBS Lett. 389:44-47, 1996; and Jefferson, EMBO J. 6:3901-3907, 1997, fl-glucuronidase).

A selectable marker (or selectable gene) generally is a molecule that, when present or expressed in a cell, provides a selective advantage (or disadvantage) to the cell containing the marker, for example, the ability to grow in the presence of an agent that otherwise would kill the cell. The selection gene can encode for a protein necessary for the survival or growth of the host cell transformed with the vector.

A selectable marker can provide a means to obtain, for example, prokaryotic cells, eukaryotic cells, and/or plant cells that express the marker and, therefore, can be useful as a component of a vector of the disclosure. The selection gene or marker can encode for a protein necessary for the survival or growth of the host cell transformed with the vector. One class of selectable markers are native or modified genes which restore a biological or physiological function to a host cell (e.g., restores photosynthetic capability or restores a metabolic pathway). Other examples of selectable markers include, but are not limited to, those that confer antimetabolite resistance, for example, dihydrofolate reductase, which confers resistance to methotrexate (for example, as described in Reiss, Plant Physiol. (Life Sci. Adv.) 13:143-149, 1994); neomycin phosphotransferase, which confers resistance to the aminoglycosides neomycin, kananmycin and paromycin (for example, as described in Herrera-Estrella, EMBO J. 2:987-995, 1983), hygro, which confers resistance to hygromycin (for example, as described in Marsh, Gene 32:481-485, 1984), trpB, which allows cells to utilize indole in place of tryptophan; hisD, which allows cells to utilize histinol in place of histidine (for example, as described in Hartman, Proc. Natl. Acad. Sci., USA 85:8047, 1988); mannose-6-phosphate isomerase which allows cells to utilize mannose (for example, as described in PCT Publication Application No. WO 94/20627); ornithine decarboxylase, which confers resistance to the ornithine decarboxylase inhibitor, 2-(difluoromethyl)-DL-ornithine (DFMO; for example, as described. In McConlogue, 1987, In: Current Communications in Molecular Biology, Cold Spring Harbor Laboratory ed.); and deaminase from Aspergillus terreus, which confers resistance to Blasticidin S (for example, as described in Tamrnura, Biosci. Biotechnol. Biochem. 59:2336-2338, 1995). Additional selectable markers include those that confer herbicide resistance, for example, phosphinothricin acetyltransferase gene, which confers resistance to phosphinothricin (for example, as described in White et al., Nucl. Acids Res. 18:1062, 1990; and Spencer et al., Theor. Appl. Genet. 79:625-631, 1990), a mutant EPSPV-synthase, which confers glyphosate resistance (for example, as described in Hinchee et al., BioTechnology 91:915-922, 1998), a mutant acetolactate synthase, which confers imidazolione or sulfonylurea resistance (for example, as described in Lee et al., EMBO J. 7:1241-1248, 1988), a mutant psbA, which confers resistance to atrazine (for example, as described in Smeda et al., Plant Physiol. 103:911-917, 1993), or a mutant protoporphyrinogen oxidase (for example, as described in U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells; tetramycin or ampicillin resistance for prokaryotes such as E. coli; and bleomycin, gentamycin, glyphosate, hygromycin, kanamycin, methotrexate, phleomycin, phosphinotricin, spectinomycin, dtreptomycin, streptomycin, sulfonamide and sulfonylurea resistance in plants (for example, as described in Maliga et al., Methods in Plant Molecular Biology, Cold Spring Harbor Laboratory Press, 1995, page 39). The selection marker can have its own promoter or its expression can be driven by a promoter driving the expression of a polypeptide of interest. The promoter driving expression of the selection marker can be a constitutive or an inducible promoter.

Reporter genes greatly enhance the ability to monitor gene expression in a number of biological organisms. Reporter genes have been successfully used in chloroplasts of higher plants, and high levels of recombinant protein expression have been reported. In addition, reporter genes have been used in the chloroplast of C. reinhardtii. In chloroplasts of higher plants, β-glucuronidase (uidA, for example, as described in Staub and Maliga, EMBO J. 12:601-606, 1993), neomycin phosphotransferase (nptII, for example, as described in Carrer et al., Mol. Gen. Genet. 241:49-56, 1993), adenosyl-3-adenyltransf-erase (aadA, for example, as described in Svab and Maliga, Proc. Natl. Acad. Sci., USA 90:913-917, 1993), and the Aequorea victoria GFP (for example, as described in Sidorov et al., Plant J. 19:209-216, 1999) have been used as reporter genes (for example, as described in Heifetz, Biocherie 82:655-666, 2000). Each of these genes has attributes that make them useful reporters of chloroplast gene expression, such as ease of analysis, sensitivity, or the ability to examine expression in situ. Based upon these studies, other exogenous proteins have been expressed in the chloroplasts of higher plants such as Bacillus thuringiensis Cry toxins, conferring resistance to insect herbivores (for example, as described in Kota et al., Proc. Natl. Acad. Sci., USA 96:1840-1845, 1999), or human somatotropin (for example, as described in Staub et al., Natl. Biotechnol. 18:333-338, 2000), a potential biopharmaceutical. Several reporter genes have been expressed in the chloroplast of the eukaryotic green alga, C. reinhardtii, including aadA (for example, as described in Goldschmidt-Clermont, Nucl. Acids Res. 19:4083-4089 1991; and Zerges and Rochaix, Mol. Cell Biol. 14:5268-5277, 1994), uidA (for example, as described in Sakamoto et al., Proc. Natl. Acad. Sci., USA 90:477-501, 1993; and Ishikura et al., J. Biosci. Bioeng. 87:307-314 1999), Renilla luciferase (for example, as described in Minko et al., Mol. Gen. Genet. 262:421-425, 1999) and the amino glycoside phosphotransferase from Acinetobacter baumanii, aphA6 (for example, as described in Bateman and Purton, Mol. Gen. Genet. 263:404-410, 2000).

In one embodiment the protein described herein is modified by the addition of an N-terminal strep tag epitope to aid in the detection of protein expression. In another embodiment, the protein described herein is modified at the C-terminus by the addition of a Flag-tag epitope to aid in the detection of protein expression, and to facilitate protein purification.

Affinity tags can be attached to proteins so that they can be purified from their crude biological source using an affinity technique. These include, for example, chitin binding protein (CBP), maltose binding protein (MBP), and glutathione-5-transferase (GST). The poly(His) tag is a widely-used protein tag that binds to metal matrices. Some affinity tags have a dual role as a solubilization agent, such as MBP and GST. Chromatography tags are used to alter chromatographic properties of the protein to afford different resolution across a particular separation technique. Often, these consist of polyanionic amino acids, such as a FLAG-tag. Epitope tags are short peptide sequences which are chosen because high-affinity antibodies can be reliably produced in many different species. These are usually derived from viral genes, which explain their high immunoreactivity. Epitope tags include, but are not limited to, V5-tag, c-myc-tag, and HA-tag. These tags are particularly useful for western blotting and immunoprecipitation experiments, although they also find use in antibody purification. Fluorescence tags can be used to give a visual readout of a protein. GFP and its variants are the most commonly used fluorescence tags. More advanced applications of GFP include using it as a folding reporter (fluorescent if folded, colorless if not).

In one embodiment, a therapeutic protein describe herein can be fused at the amino-terminus to the carboxy-terminus of a highly expressed protein (a fusion partner). A fusion partner may enhance the expression of the therapeutic gene. Engineered processing sites, for example, protease, proteolytic, or tryptic processing or cleavage sites, can be used to liberate the therapeutic protein from the fusion partner, allowing for the purification of the desired therapeutic protein. Examples of fusion partners that can be fused to a therapeutic gene are a sequence encoding the mammary-associated serum amyloid (M-SAA) protein, a sequence encoding the large and/or small subunit of ribulose bisphosphate carboxylase, a sequence encoding the glutathione S-transferase (GST) gene, a sequence encoding a thioredoxin (TRX) protein, a sequence encoding a maltose-binding protein (MBP), a sequence encoding any one or more of E. coli proteins NusA, NusB, NusG, or NusE, a sequence encoding a ubiqutin (Ub) protein, a sequence encoding a small ubiquitin-related modifier (SUMO) protein, a sequence encoding a cholera toxin B subunit (CTB) protein, a sequence of consecutive histidine residues linked to the 3′ end of a sequence encoding the MBP-encoding malE gene, the promoter and leader sequence of a galactokinase gene, and the leader sequence of the ampicillinase gene.

In some instances, the vectors of the present disclosure will contain elements such as an E. coli or S. cerevisiae origin of replication. Such features, combined with appropriate selectable markers, allows for the vector to be “shuttled” between the target host cell and a bacterial and/or yeast cell. The ability to passage a shuttle vector of the disclosure in a secondary host may allow for more convenient manipulation of the features of the vector. For example, a reaction mixture containing the vector and inserted polynucleotide(s) of interest can be transformed into prokaryote host cells such as E. coli, amplified and collected using routine methods, and examined to identify vectors containing an insert or construct of interest. If desired, the vector can be further manipulated, for example, by performing site-directed mutagenesis of the inserted polynucleotide, then again amplifying and selecting vectors having a mutated polynucleotide of interest. A shuttle vector then can be introduced into plant cell chloroplasts, wherein a polypeptide of interest can be expressed and, if desired, isolated according to a method of the disclosure.

Knowledge of the chloroplast or nuclear genome of the host organism, for example, C. reinhardtii, is useful in the construction of vectors for use in the disclosed embodiments. Chloroplast vectors and methods for selecting regions of a chloroplast genome for use as a vector are well known (see, for example, Bock, J. Mol. Biol. 312:425-438, 2001; Staub and Maliga, Plant Cell 4:39-45, 1992; and Kavanagh et al., Genetics 152:1111-1122, 1999, each of which is incorporated herein by reference). The entire chloroplast genome of C. reinhardtii is available to the public on the world wide web, at the URL “biology.duke.edu/chlamy_genome/-chloro.html” (see “view complete genome as text file” link and “maps of the chloroplast genome” link; J. Maul, J. W. Lilly, and D. B. Stern, unpublished results; revised Jan. 28, 2002; to be published as GenBank Ace. No. AF396929; and Maul, J. E., et al. (2002) The Plant Cell, Vol. 14 (2659-2679)). Generally, the nucleotide sequence of the chloroplast genomic DNA that is selected for use is not a portion of a gene, including a regulatory sequence or coding sequence. For example, the selected sequence is not a gene that if disrupted, due to the homologous recombination event, would produce a deleterious effect with respect to the chloroplast. For example, a deleterious effect on the replication of the chloroplast genome or to a plant cell containing the chloroplast. In this respect, the website containing the C. reinhardtii chloroplast genome sequence also provides maps showing coding and non-coding regions of the chloroplast genome, thus facilitating selection of a sequence useful for constructing a vector (also described in Maul, I. E., et al. (2002) The Plant Cell, Vol. 14 (2659-2679)). For example, the chloroplast vector, p322, is a clone extending from the Eco (Eco RI) site at about position 143.1 kb to the Xho (Xho I) site at about position 148.5 kb (see, world wide web, at the URL “biology.duke.edu/chlamy_genome/chloro.html”, and clicking on “maps of the chloroplast genome” link, and “140-150 kb” link; also accessible directly on world wide web at URL “biology.duke.edu/chlam-y/chloro/chlorol40.html”).

In addition, the entire nuclear genome of C. reinhardtii is described in Merchant, S. S., et al., Science (2007), 318(5848):245-250, thus facilitating one of skill in the art to select a sequence or sequences useful for constructing a vector.

For expression of the therapeutic polypeptide in a host, an expression cassette or vector may be employed. The expression vector will comprise a transcriptional and translational initiation region, which may be inducible or constitutive, where the coding region is operably linked under the transcriptional control of the transcriptional initiation region, and a transcriptional and translational termination region. These control regions may be native to the gene, or may be derived from an exogenous source. Expression vectors generally have convenient restriction sites located near the promoter sequence to provide for the insertion of nucleic acid sequences encoding exogenous proteins. A selectable marker operative in the expression host may be present in the vector.

The nucleotide sequences disclosed herein may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

The description herein provides that host cells may be transformed with vectors. One of skill in the art will recognize that such transformation includes transformation with circular vectors, linearized vectors, linearized portions of a vector, or any combination of the above. Thus, a host cell comprising a vector may contain the entire vector in the cell (in either circular or linear form), or may contain a linearized portion of a vector of the present disclosure.

Therapeutic Protein Expression

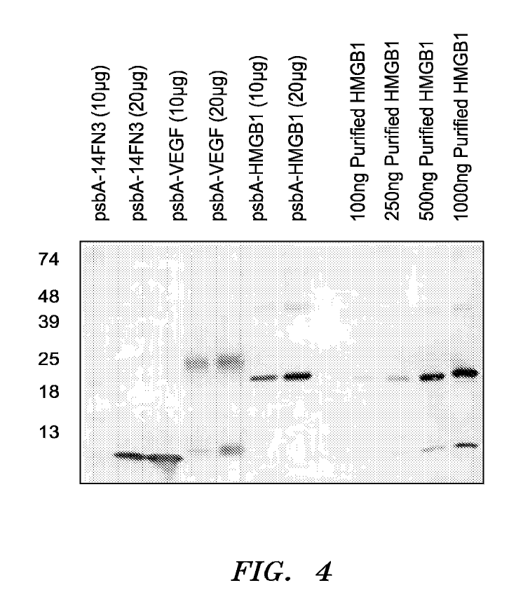

To determine percent total soluble protein, immunoblot signals from known amounts of purified protein can be compared to that of a known amount of total soluble protein lysate (for example, FIG. 4). Other techniques for measuring percent total soluble protein are known to one of skill in the art. For example, an ELISA assay or protein mass spectrometry (for example, as described in Varghese, R. S. and Ressom, H. W., Methods Mol. Bio. (2010) 694:139-150) can also be used to determine percent total soluble protein.

In some embodiments, the therapeutic compound is produced in a genetically modified host cell at a level that is at least about 0.5%, at least about 1%, at least about 1.5%, at least about 2%, at least about 2.5%, at least about 3%, at least about 3.5%, at least about 4%, at least about 4.5, or at least about 5% of the total soluble protein produced by the cell. In other embodiments, the therapeutic compound is produced in a genetically modified host cell at a level that is at least about 0.15%, at least about 0.1%, or at least about 1% of the total soluble protein produced by the cell. In other embodiments, the therapeutic compound is produced in a genetically modified host cell at a level that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, or at least about 70% of the total soluble protein produced by the cell.

Codon Optimization

As discussed above, one or more codons of an encoding polynucleotide can be “biased” or “optimized” to reflect the codon usage of the host organism. For example, one or more codons of an encoding polynucleotide can be “biased” or “optimized” to reflect chloroplast codon usage (Table A) or nuclear codon usage (Table B). Most amino acids are encoded by two or more different (degenerate) codons, and it is well recognized that various organisms utilize certain codons in preference to others. “Biased” or codon “optimized” can be used interchangeably throughout the specification. Codon bias can be variously skewed in different plants, including, for example, in alga as compared to tobacco. Generally, the codon bias selected reflects codon usage of the plant (or organelle therein) which is being transformed with the nucleic acids of the present disclosure.

A polynucleotide that is biased for a particular codon usage can be synthesized de novo, or can be genetically modified using routine recombinant DNA techniques, for example, by a site directed mutagenesis method, to change one or more codons such that they are biased for chloroplast codon usage.

Such preferential codon usage, which is utilized in chloroplasts, is referred to herein as “chloroplast codon usage.” Table A (below) shows the chloroplast codon usage for C. reinhardtii (see U.S. Patent Application Publication No.: 2004/0014174, published Jan. 22, 2004).

TABLE A
Chloroplast Codon Usage in Chlamydomonas reinhardtii

| | | | |
|---|---|---|---|
| UUU 34.1*(348**) | UCU 19.4(198) | UAU 23.7(242) | UGU 8.5(87) |
| UUC 14.2(145) | UCC 4.9(50) | UAC 10.4(106) | UGC 2.6(27) |
| UUA 72.8(742) | UCA 20.4(208) | UAA 2.7(28) | UGA 0.1(1) |
| UUG 5.6(57) | UCG 5.2(53) | UAG 0.7(7) | UGG 13.7(140) |
| CUU 14.8(151) | CCU 14.9(152) | CAU 11.1(113) | CGU 25.5(260) |
| CUC 1.0(10) | CCC 5.4(55) | CAC 8.4(86) | CGC 5.1(52) |
| CUA 6.8(69) | CCA 19.3(197) | CAA 34.8(355) | CGA 3.8(39) |
| CUG 7.2(73) | CCG 3.0(31) | CAG 5.4(55) | CGG 0.5(5) |
| AUU 44.6(455) | ACU 23.3(237) | AAU 44.0(449) | AGU 16.9(172) |
| AUC 9.7(99) | ACC 7.8(80) | AAC 19.7(201) | AGC 6.7(68) |
| AUA 8.2(84) | ACA 29.3(299) | AAA 61.5(627) | AGA 5.0(51) |
| AUG 23.3(238) | ACG 4.2(43) | AAG 11.0(112) | AGG 1.5(15) |
| GUU 27.5(280) | GCU 30.6(312) | GAU 23.8(243) | GGU 40.0(408) |
| GUC 4.6(47) | GCC 11.1(113) | GAC 11.6(118) | GGC 8.7(89) |
| GUA 26.4(269) | GCA 19.9(203) | GAA 40.3(411) | GGA 9.6(98) |
| GUG 7.1(72) | GCG 4.3(44) | GAG 6.9(70) | GGG 4.3(44) |

*Frequency of codon usage per 1,000 codons.
**Number of times observed in 36 chloroplast coding sequences (10,193 codons).

The chloroplast codon bias can, but need not, be selected based on a particular organism in which a synthetic polynucleotide is to be expressed. The manipulation can be a change to a codon, for example, by a method such as site directed mutagenesis, by a method such as PCR using a primer that is mismatched for the nucleotide(s) to be changed such that the amplification product is biased to reflect chloroplast codon usage, or can be the de novo synthesis of polynucleotide sequence such that the change (bias) is introduced as a consequence of the synthesis procedure.

In addition to utilizing chloroplast codon bias as a means to provide efficient translation of a polypeptide, it will be recognized that an alternative means for obtaining efficient translation of a polypeptide in a chloroplast is to re-engineer the chloroplast genome (e.g., a C. reinhardtii chloroplast genome) for the expression of tRNAs not otherwise expressed in the chloroplast genome. Such an engineered algae expressing one or more exogenous tRNA molecules provides the advantage that it would obviate a requirement to modify every polynucleotide of interest that is to be introduced into and expressed from a chloroplast genome; instead, algae such as C. reinhardtii that comprise a genetically modified chloroplast genome can be provided and utilized for efficient translation of a polypeptide according to any method of the disclosure. Correlations between tRNA abundance and codon usage in highly expressed genes is well known (for example, as described in Franklin et al., Plant J. 30:733-744, 2002; Dong et al., J. Mol. Biol. 260:649-663, 1996; Duret, Trends Genet. 16:287-289, 2000; Goldman et. al., J. Mol. Biol. 245:467-473, 1995; and Komar et. al., Biol. Chem. 379:1295-1300, 1998). In E. coli, for example, re-engineering of strains to express under-utilized tRNAs resulted in enhanced expression of genes which utilize these codons (see Novy et al., in Novations 12:1-3, 2001). Utilizing endogenous tRNA genes, site directed mutagenesis can be used to make a synthetic tRNA gene, which can be introduced into chloroplasts to complement rare or unused tRNA genes in a chloroplast genome, such as a C. reinhardtii chloroplast genome.

Generally, the chloroplast codon bias selected for purposes of the present disclosure, including, for example, in preparing a synthetic polynucleotide as disclosed herein reflects chloroplast codon usage of a plant chloroplast, and includes a codon bias that, with respect to the third position of a codon, is skewed towards A/T, for example, where the third position has greater than about 66% AT bias, or greater than about 70% AT bias. In one embodiment, the chloroplast codon usage is biased to reflect alga chloroplast codon usage, for example, C. reinhardtii, which has about 74.6% AT bias in the third codon position. An exemplary preferred codon usage in the chloroplasts of algae has been described in US 2004/0014174.

Table B exemplifies codons that are preferentially used in algal nuclear genes. The nuclear codon bias can, but need not, be selected based on a particular organism in which a synthetic polynucleotide is to be expressed. The manipulation can be a change to a codon, for example, by a method such as site directed mutagenesis, by a method such as PCR using a primer that is mismatched for the nucleotide(s) to be changed such that the amplification product is biased to reflect nuclear codon usage, or can be the de novo synthesis of polynucleotide sequence such that the change (bias) is introduced as a consequence of the synthesis procedure.

In addition to utilizing nuclear codon bias as a means to provide efficient translation of a polypeptide, it will be recognized that an alternative means for obtaining efficient translation of a polypeptide in a nucleus is to re-engineer the nuclear genome (e.g., a C. reinhardtii nuclear genome) for the expression of tRNAs not otherwise expressed in the nuclear genome. Such an engineered algae expressing one or more exogenous tRNA molecules provides the advantage that it would obviate a requirement to modify every polynucleotide of interest that is to be introduced into and expressed from a nuclear genome; instead, algae such as C. reinhardtii that comprise a genetically modified nuclear genome can be provided and utilized for efficient translation of a polypeptide according to any method of the disclosure. Correlations between tRNA abundance and codon usage in highly expressed genes is well known (for example, as described in Franklin et al., Plant J. 30:733-744, 2002; Dong et al., J. Mol. Biol. 260:649-663, 1996; Duret, Trends Genet. 16:287-289, 2000; Goldman et. Al., J. Mol. Biol. 245:467-473, 1995; and Komar et. Al., Biol. Chem. 379:1295-1300, 1998). In E. coli, for example, re-engineering of strains to express underutilized tRNAs resulted in enhanced expression of genes which utilize these codons (see Novy et al., in Novations 12:1-3, 2001). Utilizing endogenous tRNA genes, site directed mutagenesis can be used to make a synthetic tRNA gene, which can be introduced into the nucleus to complement rare or unused tRNA genes in a nuclear genome, such as a C. reinhardtii nuclear genome.

Generally, the nuclear codon bias selected for purposes of the present disclosure, including, for example, in preparing a synthetic polynucleotide as disclosed herein, can reflect nuclear codon usage of an algal nucleus and includes a codon bias that results in the coding sequence containing greater than 60% G/C content.

TABLE B
fields: [triplet] [frequency: per thousand] ([number])
Coding GC 66.30% 1^{st} letter GC 64.80% 2^{nd} letter GC 47.90% 3^{rd} letter GC 86.21%
Nuclear Codon Usage in Chlamydomonas reinhardtii

| | | | |
|---|---|---|---|
| UUU 5.0 (2110) | UCU 4.7 (1992) | UAU 2.6 (1085) | UGU 1.4 (601) |
| UUC 27.1 (11411) | UCC 16.1 (6782) | UAC 22.8 (9579) | UGC 13.1 (5498) |
| UUA 0.6 (247) | UCA 3.2 (1348) | UAA 1.0 (441) | UGA 0.5 (227) |
| UUG 4.0 (1673) | UCG 16.1 (6763) | UAG 0.4 (183) | UGG 13.2 (5559) |
| CUU 4.4 (1869) | CCU 8.1 (3416) | CAU 2.2 (919) | CGU 4.9 (2071) |
| CUC 13.0 (5480) | CCC 29.5 (12409) | CAC 17.2 (7252) | CGC 34.9 (14676) |
| CUA 2.6 (1086) | CCA 5.1 (2124) | CAA 4.2 (1780) | CGA 2.0 (841) |
| CUG 65.2 (27420) | CCG 20.7 (8684) | CAG 36.3 (15283) | CGG 11.2 (4711) |
| AUU 8.0 (3360) | ACU 5.2 (2171) | AAU 2.8 (1157) | AGU 2.6 (1089) |
| AUC 26.6 (11200) | ACC 27.7 (11663) | AAC 28.5 (11977) | AGC 22.8 (9590) |
| AUA 1.1 (443) | ACA 4.1 (1713) | AAA 2.4 (1028) | AGA 0.7 (287) |
| 0AUG 25.7 (10796) | ACG 15.9 (6684) | AAG 43.3 (18212) | AGG 2.7 (1150) |
| GUU 5.1 (2158) | GCU 16.7 (7030) | GAU 6.7 (2805) | GGU 9.5 (3984) |
| GUC 15.4 (6496) | GCC 54.6 (22960) | GAC 41.7 (17519) | GGC 62.0 (26064) |
| GUA 2.0 (857) | GCA 10.6 (4467) | GAA 2.8 (1172) | GGA 5.0 (2084) |
| GUG 46.5 (19558) | GCG 44.4 (18688) | GAG 53.5 (22486) | GGG 9.7 (4087) |

Table C lists the codon selected at each position for backtranslating the protein to a DNA sequence for synthesis. The selected codon is the sequence recognized by the tR NA encoded in the Chlamydomonas chloroplast genome when present; the stop codon (TAA) is the codon most frequently present in the chloroplast encoded genes. If an undesired restriction site is created, the next best choice according to the regular Chlamydomonas chloroplast usage table that eliminates the restriction site is selected.

TABLE C

| Amino acid | Codon utilized |
|---|---|
| F | TTC |
| L | TTA |
| I | ATC |
| V | GTA |
| S | TCA |
| P | CCA |
| T | ACA |
| A | GCA |
| Y | TAC |
| H | CAC |
| Q | CAA |
| N | AAC |
| K | AAA |
| D | GAC |
| E | GAA |
| C | TGC |
| R | CGT |
| G | GGC |
| W | TGG |
| M | ATG |
| STOP | TAA |

Percent Sequence Identity

One example of an algorithm that is suitable for determining percent sequence identity or sequence similarity between nucleic acid or polypeptide sequences is the BLAST algorithm, which is described, e.g., in Altschul et al., J. Mol. Biol. 215:403-410 (1990). Software for performing BLAST analysis is publicly available through the National Center for Biotechnoiogy Information. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (as described, for example, in Henikoff & Henikoff (1989) Proc. Natl. Acad. Sci. USA, 89:10915). In addition to calculating percent sequence identity, the BLAST algorithm also can perform a statistical analysis of the similarity between two sequences (for example, as described in Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, less than about 0.01, or less than about 0.001.

### EXAMPLES

The following examples are intended to provide illustrations of the application of the present invention. The following examples are not intended to completely define or otherwise limit the scope of the invention.

One of skill in the art will appreciate that many other methods known in the art may be substituted in lieu of the ones specifically described or referenced herein.

### Example 1

### Introduction of Recombinant Genes into the C. Reinhardtii Chloroplast Genome

The C. reinhardtii chloroplast genome shows a high AT content and noted codon bias (Franklin S., et al., (2002) Plant J 30:733-744; Mayfield S. P. and Schultz J. (2004) Plant J 37:449-458). To achieve protein expression, the genes of interest were first converted to match the codon usage of C. reinhardtii by synthesizing each of the seven genes in a codon-bias optimized for the C. reinhardtii chloroplast (see Table 2). A codon bias threshold of greater than 10% of codons normally used for that amino acid was chosen and the genes were assembled via overlapping oligonucleotides. A FLAG-tag epitope (SEQ ID NO: 60) was added to the C-terminal end of each protein sequence to allow detection by western blot and to facilitate protein purification. SEQ ID NOs: 1, 3, 5, 7, 9, 11, and 13 were used for cloning; the 5′ end of each sequence was engineered to contain an NdeI site and the 3′ end of each sequence was engineered to contain an XbaI site.

A range of endogenous promoters and UTRs were previously examined for recombinant protein expression in wild-type C. reinhardtii 137c, with the atpA and psbA promoters and UTRs showing the good expression (Barnes D., et al. (2005) Mol Genet Genomics 274:625-636). Expression from the psbA promoter has very good potential when the endogenous psbA gene product, D1, is absent (Manuell A. L., et al (2007) Plant Biotechnol J 5:402-412; Surzycki R., et al. (2009) Biologicals 37:133-138), probably due to the interruption of a negative feedback loop (Minai L., et al. (2006) Plant Cell 18:159-175). Expression from this promoter is also increased by an increase in light intensity (Manuell A. L., et al (2007) Plant Biotechnol J 5:402-412). Thus, both the atpA and the psbA promoters were chosen for the expression analysis of the recombinant genes described herein. For psbA expression the genes were cloned into the transformation vector pD1-KanR under the control of the psbA promoter and 5′ UTR and psbA 3′ UTR (SEQ D NO: 66)(FIG. 1A). SEQ ID NO: 92 contains within the sequence, the psbA promoter and 5′ UTR. SEQ ID NO: 92 is nucleotide sequence 137,316 to 138,792 of the Chlamydomonas reinhardtii genome (Gen Bank No. BK000554; Maul, J. E., et al., Plant Cell (2002) 2659-2679) and is shown in FIGS. 1A and 1B as the “5′flanking” and “psbA promoter/5′ UTR”.

The pD1-KanR vector also contains a kanamycin resistance gene (aphA6) under the control of the atpA promoter and 5′UTR (SEQ ID NO: 63) and the rbcL 3′UTR (SEQ ID NO: 67), which is cloned downstream of the psbA expression site, and is used for the selection of transformants (FIG. 1). This expression cassette contains homology to the psibA region of the C. reinhardtii chloroplast genome and thus after transformation will replace the psbA locus (and gene) by homologous recombination (Manuell A. L., et al (2007) Plant Biotechnol J 5:402-412). The resulting transformants are resistant to the antibiotic kanamycin and are psbA deficient.

Expression of the seven genes was also tested using the atpA promoter and 5′ UTR (SEQ ID NO: 63) and the rbcL 3′ UTR (SEQ ID NO: 67)(FIG. 1C). The genes were cloned into the p322 plasmid and therefore integrated into a silent site in the inverted repeat just downstream of the pshA locus (Franklin S., et al. (2002) Plant J 30:733-744). These constructs were co-transformed with the p228 plasmid conferring resistance to spectinomycin (Franklin S., et al. (2002) Plant J 30:733-744). All of the genes were also cloned into the psbA::SAA fusion plasmid, which was designed to fuse the protein of interest to the carboxy terminus of the well expressed mammalian protein M-SAA (Manuell A. L., et al (2007) Plant Biotechnol J 15:402-412). A protease cleavage site (Thrombin) between SAA and the protein of interest was engineered so that SAA could be removed during downstream processing (FIG. 1B). As in the pD1-KanR vector, the SAA-fusion constructs are under the control of the psbA promoter and UTRs, replace the endogenous psbA locus, and contain the atpA::aphA6 kanamycin resistance gene for selection.

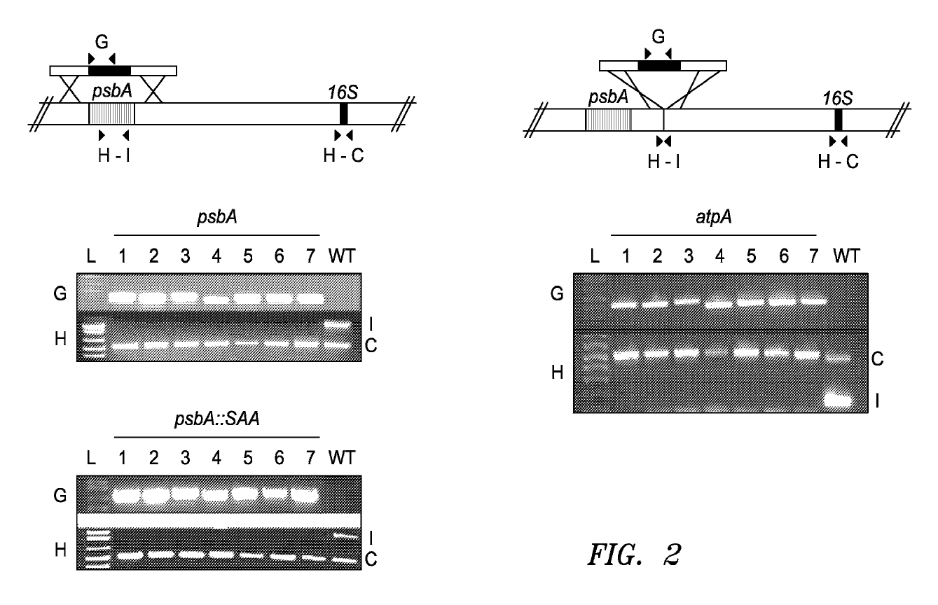

All constructs were transformed by particle bombardment into C. reinhardtii wild type strain 137c (mt+). Primary transformants were selected on media containing either kanamycin (for pD1-KanR) or spectinomycin (p228) and screened for integration and homoplasmicity by PCR (FIG. 2). Each of the seven genes, in all three constructs, was stably integrated into the chloroplast genome (G, FIG. 2). Homoplasmic cell lines, in which all copies of the chloroplast genome contained the recombinant gene, were isolated through multiple rounds of streaking for single colonies under antibiotic resistance selection. Colony PCR screening was used to confirm that strains were homoplasmic for the correct gene integration (H-I, FIG. 2). Efficiency of transformation (number of gene positive colonies/number of colonies) with the construct containing the kanamycin cassette in cis with the gene of interest was much greater than that seen with other co-transformation protocols.

### Example 2

### Accumulation of Recombinant Proteins in Transgenic Chloroplast

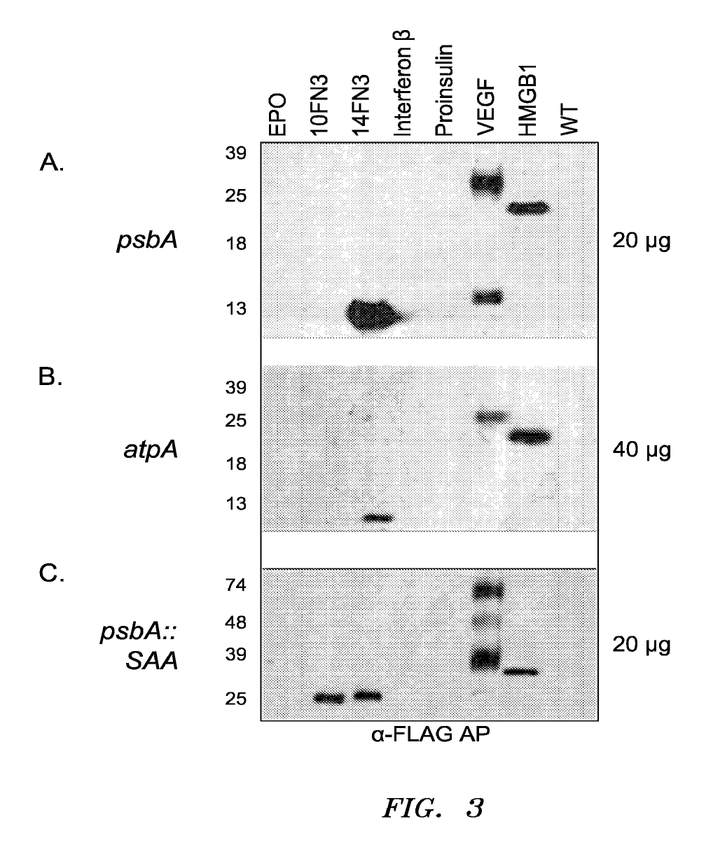

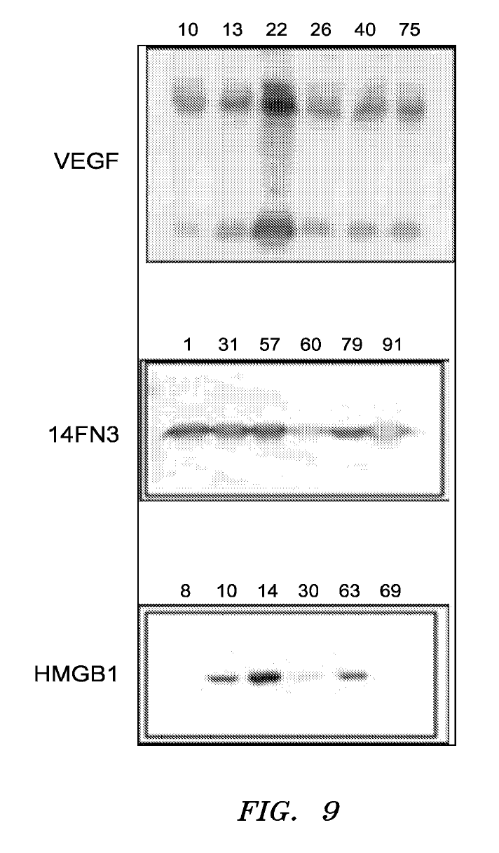

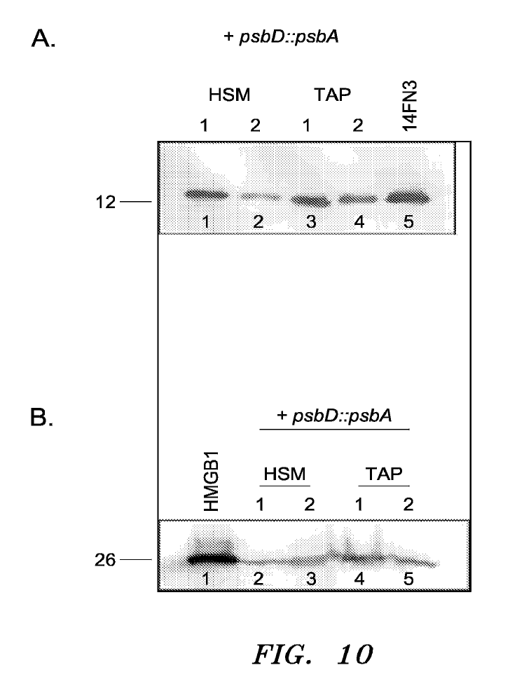

Six homoplasmic cell lines for each of the recombinant genes were isolated and approximate protein expression levels were determined by western blotting. Protein expression was relatively consistent for each of the homoplasmic lines isolated for each gene (FIG. 9), and only one transgenic line for each protein was characterized in detail, and shown in FIG. 3. Proteins 14FN3, VEGF, and HMGB1 show significant expression when the corresponding gene is expressed from the psbA promoter (FIG. 3A). Moreover, all three proteins were soluble. Expression from this promoter was also induced by a shift from dark or dim light into bright light, as has previously been reported for other recombinant genes expressed from the psbA promoter (Barnes D., et al. (2005) Mol Genet Genomics 274:625-636; Manuell A. L., et al (2007) Plant Biotechnol J 5:402-412). Native VEGF is active as a dimer (Potgens A. J., et al. (1994) J Biol Chem 269:32879-32885) and even under the denaturing conditions of SDS-PAGE Chlamydomonas expressed VEGF appears to show dimerization (FIG. 3 and FIG. 9), suggesting proper protein folding. 14FN3, VEGF, and HMGB1 accumulated to approximately 3%, 2% and 2.5%, respectively, of total soluble protein when expressed using the psbA promoter (FIG. 4). This represents a level of expression high enough to allow for relatively easy purification of the proteins. When the psbA gene under the control of the psbD promoter was reintroduced by particle bombardment into the 31HB silent site (Manuell A. L., et al (2007) Plant Biotechnol J 5:402-412), protein levels were only slightly reduced while photosynthesis was completely restored (FIG. 10). Thus, high levels of recombinant protein expression are maintained under photosynthetic growth conditions.

To test whether the remaining proteins were expressed at levels below that detectable by western blotting of total soluble protein, immunoprecipitations were performed from 50 ml liquid cultures using anti-FLAG chromatography resin (Sigma). Using this technique, low levels of expression of 10FN3 and proinsulin were observed, but no detectable protein accumulation for interferon β or EPO.

When each of the genes was expressed from the alpA promoter, the same three proteins (14FN3, VEGF, and HMGB1) accumulated, however to significantly lower levels than when expressed from the psbA promoter (FIG. 3B). Under the atpA promoter, 14FN3, VEGF, and HMGB1 accumulated to approximately 0.15%, 0.1% and 1% of total soluble protein. Thus, both promoters support expression of the same three proteins, but the psbA promoter and UTRs drives recombinant protein accumulation up to twenty times greater than that from the atpA promoter and 5′ UTR.

### Example 3

### Plasmid Construction

Codon optimization for C. reinhardtii chloroplast expression was performed using software specifically designed for polymerase cycling assembly (PCA)-based de-novo gene synthesis. This program generates gene sequences by the simultaneous optimization of multiple parameters: normalization of the codon distribution to that of the C. reinhardtii chloroplast (data obtained from http://www.kazusa.or.jp/codon (Nakamura Y., et al. (2000) Nucleic Acids Res 28:292)); uniformity of physical properties of the output oligonucleotides (GC content, melting temperature, length); and avoidance of unfavorable mRNA structures. The seven genes were assembled by PCA using sense and antisense oligonucleotides ranging in length from 51 to 63 bases, sharing eighteen base pairs of overlapping sequence homology (Minshull J., et al. (2004) Methods 32:416-427). The number of oligonucleotides used ranged from eight oligos for proinsulin to sixteen oligos for HMGB1.

Each gene was constructed with an NdeI restriction site at the 5′ end and a XbaI site at the 3′ end of the coding region (SEQ ID NOs: 1, 3, 5, 7, 9, 11, and 13), along with a C-terminal TEV protease recognition site (ENLYFQG) (SEQ ID NO: 62) and a FLAG-tag (SEQ ID NO: 60). The genes were directionally cloned into the pD1-KanR vector, constructed by the addition of the Kanamycin resistance gene aphA6 (Acinetobacter baumannii) into the unique BamHI site in the psbA vector described previously (Manuell A. L., et al (2007) Plant Biotechnol J 5:402-412). The coding sequence of aphA6 was ordered in C. reinhardtii chloroplast codon bias (nucleotide sequence is SEQ ID NO: 75; amino acid sequence is SEQ ID NO:76) from DNA2.0 (www.dna20.com; DNA2.0 Headquarter, 1430 O'Brien Drive, Suite E, Menlo Park, Calif. 94025, USA) flanked by an atpA 5′ promoter and UTR (SEQ ID NO: 63) and a rbcL 3′ UTR (SEQ ID NO: 67) (Barnes D., et al. (2005) Mol Genet Genonmics 274:625-636). For constructs containing the atpA promoter, the genes of interest were cloned into the unique NdeI/XbaI restriction sites in p322 vector (Franklin S., et al. (2002) Plant J 30:733-744).

CAI values were determined with the CAI calculator (http://genomes.urv.cat/CAIcal/ Puigbo, P., et al., (2008) CAIcal: a combined asses codon usage adaptation. Biology Direct, 3:38) using the C. reinhardtii chloroplast codon usage table (http://www.kazusa.or.jp/codon/cgi-bin/showcodon.cgi?species=3055.chloroplast; reproduced below). CAI values range from 0 to 1, with 1 being if a gene always uses the most frequently used codon of a reference set (Puigbo P., et al. (2008) BMC Bioinformatics 9:65).

TABLE 2
CODON USAGE TABLE -
chloroplast Chlamydomonas reinhardtii [gbpln]: 93 CDS's
(26731 codons)
fields: [triplet] [frequency: per thousand] ([number])

| | | | |
|---|---|---|---|
| UUU 33.4(894) | UCU 17.0(455) | UAU 24.6(657) | UGU 7.6(203) |
| UUC 17.1(456) | UCC 2.8(74) | UAC 10.0(266) | UGC 1.5(39) |
| UUA 77.7(2078) | UCA 22.0(588) | UAA 2.9(78) | UGA 0.1(3) |
| UUG 4.3(114) | UCG 4.0(107) | UAG 0.4(12) | UGG 13.5(361) |
| CUU 14.3(383) | CCU 15.5(414) | CAU 10.1(270) | CGU 32.4(866) |
| CUC 1.0(28) | CCC 3.4(90) | CAC 8.8(235) | CGC 4.1(110) |
| CUA 6.4(170) | CCA 23.6(630) | CAA 38.4(1026) | CGA 3.4(90) |
| CUG 3.7(99) | CCG 2.4(63) | CAG 4.1(110) | CGG 0.5(14) |
| AUU 51.4(1374) | ACU 24.4(651) | AAU 42.1(1126) | AGU 16.0(428) |
| AUC 8.2(219) | ACC 5.1(135) | AAC 17.7(472) | AGC 5.4(144) |
| AUA 6.9(184) | ACA 32.4(865) | AAA 69.1(1847) | AGA 5.3(143) |
| AUG 22.3(596) | ACG 3.9(103) | AAG 6.2(167) | AGG 0.9(23) |
| GUU 29.3(783) | GCU 34.0(908) | GAU 25.3(676) | GGU 44.0(1177) |
| GUC 2.5(68) | GCC 5.9(159) | GAC 9.8(263) | GGC 6.4(172) |
| GUA 26.0(696) | GCA 20.7(554) | GAA 41.1(1098) | GGA 8.6(229) |
| GUG 5.6(149) | GCG 3.3(88) | GAG 5.7(152) | GGG 3.7(99) |

Coding GC 33.72% 1st letter GC 44.40% 2nd letter GC 37.35% 3rd letter GC 19.40%.

### Example 4

### C. reinhardtii Strains, Transformations and Growth Conditions

For chloroplast transformations, C. reinhardtii wt strain 137c (mt+) was grown to late logarithmic phase in the presence of 40 mM 5-fluorodeoxyyuridine in TAP (Tris-acetate-phosphate) medium (Gorman D. S. and Levine R. P. (1965) Proc Natl Acad Sci U S A 54:1665-1669) at 23° C. under constant illumination of 5000 lux on a rotary shaker. Cells were harvested by centrifugation and resuspended in TAP medium. Approximately 5×10⁷ cells/plate were spread onto TAP/agar plates containing the appropriate antibiotic. Chloroplast transformations were performed by particle bombardment (Boynton J. E., et al. (1988) Science 240:1534-1538) using DNA-coated gold particles (S550d, Seashell Technologies, San Diego, Calif.). The following bombardment parameters were used with the PDS-1000/He system (BioRad, Hercules, Calif.): chamber vacuum of 27-28 inches Hg, target distance of 9 cm, helium pressure of 1350 psi, and approximately 1 mg of 0.55 μm gold coated with 3 μg of DNA per transformation plate. Transformations with the psbA promoter were carried out using kanamycin selection (100 μg/ml in TAP agar, 150 μg/ml for propagation after transformation), for which resistance was conferred by the kanamycin resistance gene aphA6 expressed from the same construct under the control of the atpA promoter/5′ UTR and rbcl 3′ UTR. The recombinant genes under the control of the atpA promoter were co-transformed with the plasmid p228 (Chlamydomonas Stock Center, Duke University, Durham, N.C., USA), which contains a point mutation in the 16S rRNA gene that confers spectinomycin resistance. Chloroplast transgenic lines were identified by growth on media containing spectinomycin (150 μg/ml TAP agar).

### Example 5

### PCR Screening of Transformants

Primary transformants were screened for the presence of the gene of interest using promoter specific forward primers (SEQ ID NOs: 35 and 36) and gene specific reverse primers (SEQ ID NOs: 37-43). Cells were resuspended in Tris-EDTA solution and heated to 95° C. for 10 minutes. The cell lysate was then used as a template for PCR under standard conditions using Taq polymerase (NEB, Ipswich, Mass.). For assessing homoplasmicity of clones, the PCR primers 5′-GGAAGGGGAGGACGTAGGTACATAAA-3′ (SEQ ID NO: 29) and 5′-TTAGAACGTGTTTTGTTCCCAAT-3′ (SEQ ID NO: 30) were used for constructs incorporated at the psbA site and 5′-CCCATAAATAAAAGTTTCAATTGG-3′ (SEQ ID NO: 31) and 5′-CGGTGGTTATTCCAGGCCAAACTTATG-3′ (SEQ ID NO: 32) for constructs incorporated at the p322 site. Each set of primers anneal to regions that are disrupted upon gene insertion through homologous recombination. Thus, the loss of a PCR product indicates proper gene integration into all copies of the chloroplast genome. For these reactions, a second set of control primers were used (5′-CCGAACTGAGGTTGGGTTTA-3′ (SEQ ID NO: 33) and 5′-GGGGGAGCGAATAGGATTAG-3′ (SEQ ID NO: 34)). These primers amplify a region of the genome away from the recombinant gene insertion site and serve as a positive control in the multiplex PCR. Results of the screen are shown in FIG. 2.

### Example 6

### Western Blotting

Whole cell samples were resuspended in lysis buffer (Franklin S., et al. (2002) Plant J 30:733-744) and lysed by sonication. Total soluble protein was isolated by centrifugation and denatured by the addition of SDS-PAGE loading buffer (Laemelli) followed by incubation at 60° C. for 10 minutes. When protein determination was required, a sample was taken prior to the addition of SDS-PAGE sample buffer and protein concentration determined using the Bio-Rad DC protein assay as per the manufacturer's instructions (Bio-Rad). Proteins were separated on 12% or 16% SDS-page gels at 120-150 volts unless otherwise stated and transferred to nitrocellulose membrane at 200 mAmps for 1.5 hours. After blocking with 5% milk, membranes were probed with an anti-FLAG monoclonal antibody conjugated to HRP (A8592, Sigma, St. Louis, Mo.) or to alkaline phosphatase (A9469, Sigma). Western blot results are shown in FIG. 3.

### Example 7

### Protein Purification

One to two liters of algal cells grown to late log phase in TAP media were collected by centrifugation at 5000×G for 10 minutes. The cell paste was resuspended to a volume of 40-100 ml per liter of culture in lysis buffer, 50 mM Tris pH 8.0, 400 mM NaCl, 0.1% Tween 20, 1 mM phenylmethylsulfonylfluoride (PMSF), and lysed by sonication. The lysate was clarified by centrifuigation at 30,000×G for 20 minutes, and the supernatant was collected. One to two mls of anti-FLAG M2 resin (Sigma) was added to the clarified lysate, and rotated end over end at 4° C. for four hours. The anti-FLAG beads were collected by filtration in a Bio-Rad Econo-pac column, and washed extensively with lysis buffer. The protein was eluted from the resin using lysis buffer containing 100 micrograms per ml FLAG peptide (Sigma) or with 100 mM glycine pH 3.5, 400 mM NaCl and neutralized with Tris pH 7.9 to final a concentration of 50 mM. After adding five column volumes of elution buffer, the column was incubated at 4° C. overnight, under rotation. Fractions were collected and assayed by SDS-PAGE followed by western blot and coomassie staining to determine the fractions that contained the recombinant protein. Fractions containing recombinant protein were pooled and concentrated using an Amicon Ultra centrifugal filter with a molecular weight cut-off of 5 kDa (Millipore, Billerica, Mass.). After concentration, samples were checked again by SDS-PAGE, and concentrations were determined using the BCA protein assay (Bio-Rad) with bovine serum albumin as a standard.

### Example 8

### RT-Quantitative PCR Analysis of mRNA Accumulation

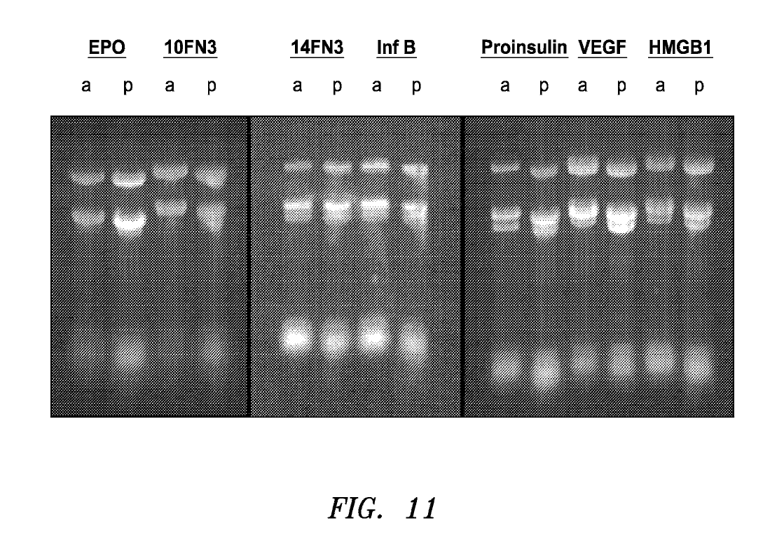

Cells were grown in 50 ml liquid TAP under 1000 lux of light illumination until mid to late log phase. 10 mis of cells were harvested by centrifugation and total RNA was purified using the Plant RNA Reagent (Invitrogen, Carlsbad, Calif.) as per the manufacturer's instructions for small scale purification. RNA integrity was monitored by agarose gel electrophoresis (FIG. 11). 10 μg of total RNA was treated with DNase to remove any contaminating genomic DNA (Ambion DNA-free, Austin, Tex.). 400 ng of DNase-treated total RNA was then used for first strand cDNA synthesis using Bio-Rad's iScript cDNA Synthesis kit (following the manufacturer's instructions). The thermocycling conditions used are as follows: 5 minutes at 25° C., 30 minutes at 42° C., 5 minutes at 85° C., hold at 4° C. Reactions were also carried out in the absence of reverse transcriptase as a control for genomic DNA contamination. cDNAs were either diluted 1:10 for the qPCR experimental reactions, or diluted 1:4 and then subjected to a 4-fold serial dilution to determine PCR efficiencies for each primer pair (SEQ ID NOs: 44-57). 6.5 μl of diluted cDNA was used in a 25 μl qPCR reaction using Bio-Rad SYBR Green Supermix and 0.5 μM oligonucleotides. Real-time qPCR was carried out in triplicate for each sample in a Bio-Rad My iQ thermocycler performing 40 cycles of a two-step protocol with an annealing/extension temperature of 55° C., followed by a melt curve to monitor for primer dimers. For all qPCR reactions, rbcL was used as the control gene (primers are SEQ ID NOs: 58 and 59). Relative mRNA levels were determined using the Pfaffl method (ratio=Ê−dCt_{target}/Ê−dCt_{control}) (Pfaffl M. W. (2001) Nucleic Acids Res 29:e45).

In an exemplary embodiment, the general versatility of algae as a platform for therapeutic protein production was tested by examining the expression of seven recombinant human proteins in the chloroplast of Chlamydomonas reinhardtii. The seven proteins are either presently sold as therapeutics, or have the potential to become human therapeutics, and each protein was tested using three different expression strategies. Protein expression and mRNA levels were quantitatively compared for each protein. Some level of expression was observed for five of the seven proteins, and three of the proteins accumulated to levels above 1% of total soluble protein, levels sufficient for easy purification. Another protein accumulated to these same high levels when fused to a stable and highly expressed protein, M-SAA, and an additional protein could be detected by immunoprecipitation, but accumulated at very low levels. Importantly, all four of the highly expressed proteins were soluble, with no evidence that any of them were found in insoluble aggregates or inclusion bodies. While the highest levels of recombinant protein accumulation were found to occur in the non-photosynthetic psbA null background, restoration of photosynthesis by reintroduction of the psbA gene under the control of the psbD promoter (Manuell A. L., et al (2007) Plant Biotechnol J 5:402-412), resulted in photosynthetic strains with recombinant protein levels only slightly reduced compared to the non-photosynthetic strains. Thus, high levels of recombinant protein accumulation can be achieved under photosynthetic growth conditions, which is required for some of the economic advantages algae holds over other expression systems. Although this is a small number of proteins, it is a diverse set of protein types, and this level of success is much greater then the 20% to 30% success rate of human and viral proteins expressed and were soluble in bacteria Alzari P. M., et al., (2006) Acta Crystallogr D Biol Crystallogr 62:1103-1113), and equivalent to the 45% (Banci L., et al. (2006) Acta Crystalogr D Biol Crystallogr 62:1208-1217) to 58% (Aricescu A. R., et al. (2006) Acta Crystallogr D Biol Crystallogr 62:1114-1124) success rate reported for recombinant protein expression in other eukaryotic systems.

VEGF and HMGB1 accumulated to 3% and 2.5% respectively, and these proteins were purified using affinity chromatography to the FLAG epitope added to the carboxy terminus of each protein. Using standard bioactivity assays both proteins were found to have similar activity as the same proteins expressed in bacteria, the system presently used for production of these two proteins for therapeutic use. The yields reported here are close to the yields reported for therapeutic proteins expressed from the chloroplast of higher plants. For example, human serum albumin was reported to accumulate to 11% TSP (Fernandez-San Millan A., et al. (2003) Plant Biotechnol J 1:71-79), somatotropin to 7% TSP (Staub J. M., et al. (2000) Nat Biotechnol 18:333-338), interferon gamma to 6% TSP (Leelavathi S, and Reddy, V. S. (2003) Molecular Breeding 11:49-58), and a CTB-proinsulin fusion to 16% TSP (Ruhlman T., et al. (2007) Plant Biotechnol J 5:495-510). These data confirm that algal chloroplasts are able to produce bioactive proteins, and that the proteins can be easily purified from algal extracts.

Some proteins accumulate in algae while others do not. Protein expression is variable in all expression platforms and algae are not unique in that regard. The greater than 50% expression found in these studies is actually much higher then the percent of protein expressed in bacterial systems (Alzari P. M., et al., (2006) Acta Crystallogr D Biol Crystallagr 62:1103-1113), and comparable with the best expression reported for other eukaryotic systems (Aricescu A. R., et al. (2006) Acta Crystallogr D Biol Crystallogr 62:1114-1124; Banci L., et al. (2006) Acta Crystallogr D Biol Crystal/ogr 62:1208-1217). RT-qPCR analysis revealed that mRNA transcripts accumulated for all recombinant genes tested, and there was a poor correlation between transcript level and protein accumulation, suggesting that transcription and mRNA accumulation may not determine the level of recombinant protein accumulation in algae. It was also observed that the proteins that expressed with the atpA promoter and UTR were the same proteins that expressed with the psbA promoter, suggesting that failure to accumulate these proteins is not determined by the promoter or UTRs used. Thus, either the proteins that express poorly are highly unstable, or their coding regions somehow precluded translation of the chimeric mRNAs.

Although accumulation of recombinant proteins in algae at 2% to 3% of total soluble protein is sufficient for economic production, higher levels of accumulation would obviously reduce cost even more, and would also likely improve protein purification efficiency. The data described herein also shows that the psbA promoter/UTR showed better protein expression for all the proteins tested compared to the atpA promoter/UTR, and that this increase does not correlate directly with increased mRNA accumulation. This data suggest that translation of chimeric mRNAs containing the psbA UTR is better than translation of mRNAs with the same coding but containing the atpA UTR. Thus, it is possible that altering UTRs may further improve protein translation as a way to increase protein accumulation.

### Example 9

### Fusion to M-SAA Protein Increases Accumulation of Fibronectin Domain 10

One possible explanation for the lack of protein accumulation of 10FN3, proinsulin, interferon β, and EPO is protein instability. Fusion of poorly expressed proteins to a well-expressed and stable protein has been shown to increase the accumulation of the former in many expression systems, including bacteria (Butt T. R., et al., (2005) Protein Expr Purif 43:1-9; De Marco V., et al. (2004) Biochem Biophys Res Conmmun 322:766-771; Pryor K D and Leiting B (1997) Protein Expr Purif 10:309-319; Sachdev D and Chirgwin J M (2000) Methods Enzymol 326:312-321; Wang C., et al. (1999) Biochem J 338 (Pt 1):77-81) and plant and algal chloroplasts (Streatfield, S. J. (2007) Plant Biotechnol J 5:2-15; Muto M., et al. (2009) BMC Biotechnol 9:26). Indeed, expression of human proinsulin in E. coli and yeast was facilitated by the construction of fusion proteins (Chan S. J., et al. (1981) Proc Natl Acad Sci U S A 78:5401-5405; Stepien P. P., et al. (1983) Gene 24:289-297). Human proinsulin fusions have been expressed from the plant nuclear genomes of potato tubers at 0.1% TSP (Arakawa T., et al. (1998) Nat Biotechnol 16:934-938) and Arabidopsis at 0.1% total seed protein (Nykiforuk C. L., et al. (2006) Plant Biotechnol J 4:77-85), and in the chloroplast of tobacco and lettuce at ˜16% and ˜2.5% TSP, respectively (Ruhiman T., et al., (2007) Plant Biotechnol J 5:495-510). To test whether this same effect could work in algal chloroplasts, each of the recombinant genes was cloned as a fusion partner to the gene encoding the mammary-associated serum amyloid protein (M-SAA).

It has been shown that expression of a mammalian serum amyloid protein (M-SAA) in C. reinhardtii chloroplast to about 10% of TSP was obtained by using the psbA promoter and UTRs in a targeted psbA knockout strain (Manuell A. L., et al (2007) Plant Biotechnol J 5:402-412). When the psbA gene was reintroduced elsewhere in the genome under the control of the psbD promoter, photosynthesis was restored while M-SAA protein levels were only slightly reduced, showing that photosynthetic competent algae can produce high levels of recombinant proteins (Manuell A. L., et al (2007) Plant Biotechnol J 5:402-412). Furthermore, the purified protein was found to have bioactivity similar to the authentic, naturally occurring protein, demonstrating the usefulness of the system as a robust platform for the production of recombinant proteins.

M-SAA fusion constructs were placed under the control of the psbA promoter and UTRs (FIG. 1B) and transformed as above, selecting for transformants by kanamycin resistance. Western blots of total soluble protein revealed that fusions of 14FN3, VEGF and HMGB1 to NM-SAA led to significant protein accumulation; the same three proteins that accumulated using the psbA and atpA promoters alone. In addition, the fusion of fibronectin 10FN3 to M-SAA enabled significant protein accumulation to occur, and expression levels similar to those achieved with the three other proteins was observed (FIG. 3C). Interestingly, the expression of HMGB1, which was substantial without fusion to M-SAA, actually showed decreased accumulation when fused to the SAA protein, from 2.5% to approximately 1% of total soluble protein.

### Example 10

### Accumulation of Recombinant mRNAs from Different Promoters

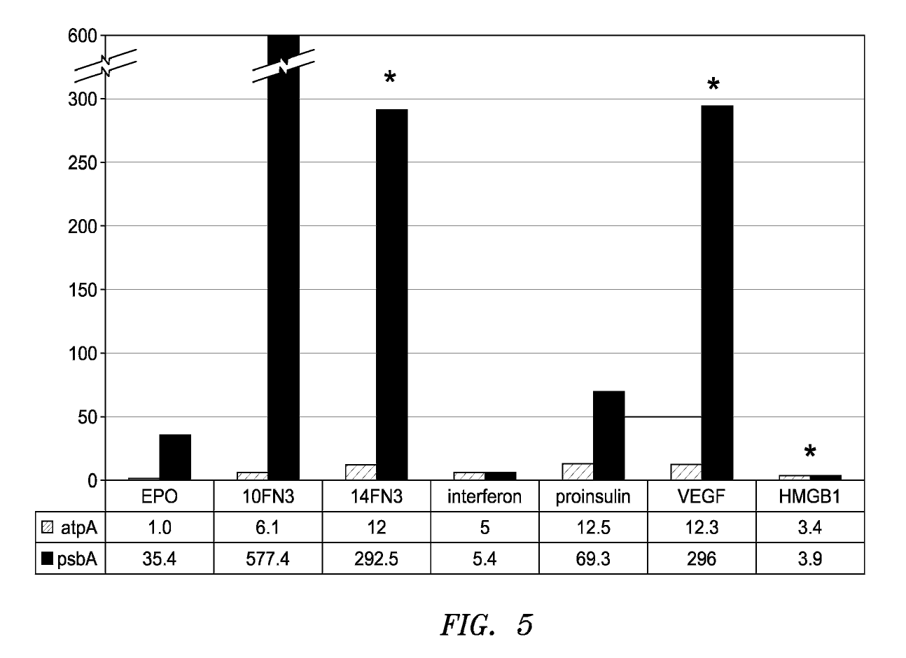

The regulation of endogenous chloroplast gene expression primarily occurs at the level of translation (Zerges W (2000) Translation in chloroplasts. Biochimie 82:583-601). To address the relationship between translation and transcription in recombinant gene expression, reverse transcriptase quantitative PCR (RT-qPCR) was used to determine the level of recombinant mRNAs for each of the seven genes under the control of the psbA and atpA promoters (FIG. 5). Total RNA was isolated from liquid cultures grown in 1000 lux of light illumination (FIG. 11). Following cDNA synthesis, SYBR green based qPCR was performed using gene-specific primers (SEQ ID NOs: 44-59). qPCR signals were detectable for all 14 constructs, indicating that stable mRNA transcripts accumulated for all of the recombinant genes (FIG. 5). In general, the psbA promoter yielded higher levels of mRNA transcript than the atpA promoter. Interestingly, while 14FN3, VEGF, and HMGB1 protein accumulated to approximately equal levels (3%, 2% and 2.5% respectively of total soluble protein; FIG. 4), the psbA-HMGB1 mRNA transcript was found to be approximately 75-fold less abundant than psbA-14FN3 and psbA-VEGF mRNA transcripts (FIG. 5). Overall there was a poor correlation between mRNA accumulation and protein accumulation, as has been reported for endogenous chloroplast genes (Eberhard et al., 2002; Nickelsen, 2003; Zerges, 2000). However in this study, in no case was the lack of protein accumulation caused by a lack of mRNA accumulation.

### Example 11

### Purification of Bio-Active Recombinant Proteins from Chlamydomonas

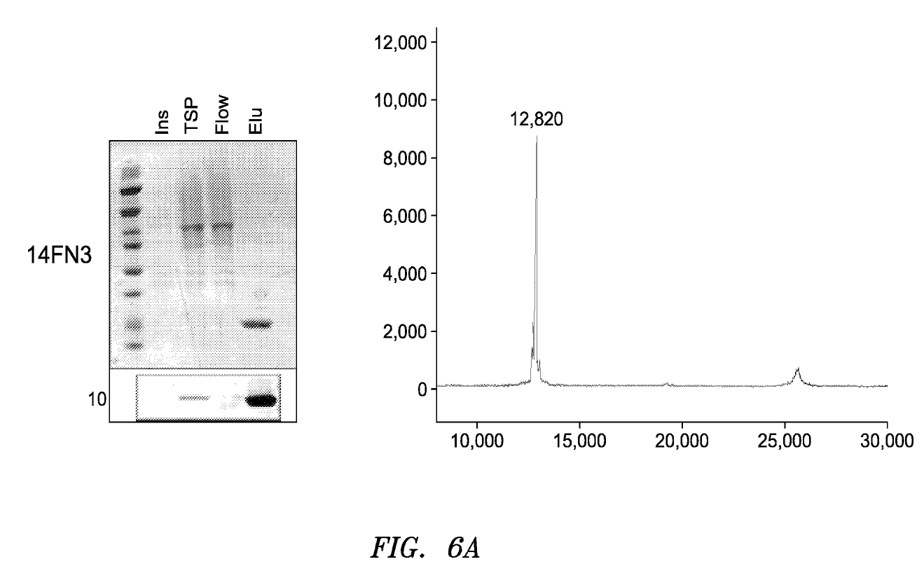

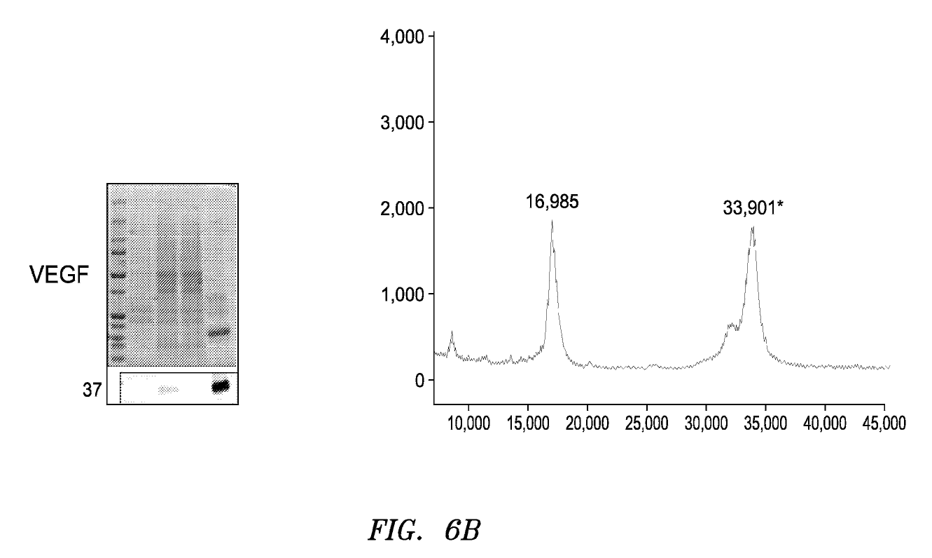

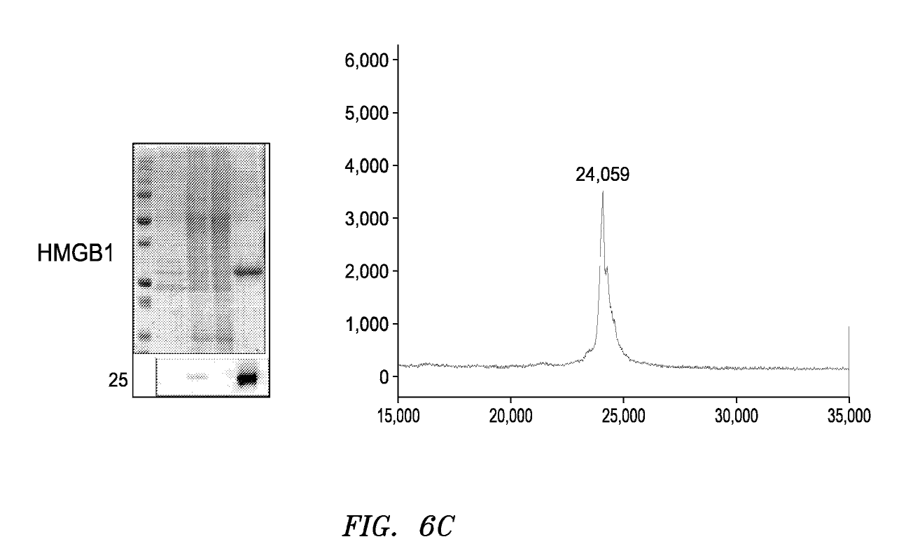

To become a viable protein production platform, algal chloroplasts must not only express recombinant proteins, but those proteins must be biologically active in a highly purified state. 14FN3, VEGF, and 1HMGB1 were affinity purified using FLAG affinity chromatography to the C-terminal FLAG epitope. Western blotting of samples taken throughout the purification processes indicates that all detectable protein was found in the soluble fractions of the cell lysates (TSP, FIGS. 6A-C). Thus, most, if not all, of the recombinant protein is soluble. Furthermore, the FLAG-tagged proteins efficiently bound to the resin as indicated by little to no detectable protein in the column flow-through, allowing for ease of purification and good recovery (Flow, FIGS. 6A-C). Finally, coomassie staining of purified 14FN3 and HMGB1 each revealed a single predominant band at approximately the predicted molecular weight. BioRad Precision Plus ladder was used (BioRad, USA). Coomassie staining of purified VEGF revealed a single predominant band around 16 kDa, the predicted size of the monomer, with a faint band at approximately 30 kDa, the expected mass of the VEGF dimer. 14FN3 has a predicted molecular mass of 12,820 mass units. Algal-expressed 14FN3 has a mass average of 12,820 and appears predominantly as a single peak in matrix-assisted laser desorption-ionization time-of-flight mass spectrometry (MALDI-TOF MS) analysis (FIG. 6A). HMGB1 aa13-169 has a predicted molecular mass of 24,036 mass units. The MALDI-TOF MS analysis of algal-expressed HMGB1 shows predominately a single peak at 24,059, just 23 mass units off the predicted value (FIG. 6C). Two peaks appear in the VEGF MALDI-TOF MS analysis (FIG. 6B), a mass average value of 16,985 and 33,901. The predicted value of a VEGF monomer is 17,064, and 34,128 for the dimer. Thus the two peaks mostly likely represent the monomer and dimer of VEGF. Together, these data show that the algal-expressed proteins accumulate in a soluble form, can be purified to high purity, and are largely unmodified.

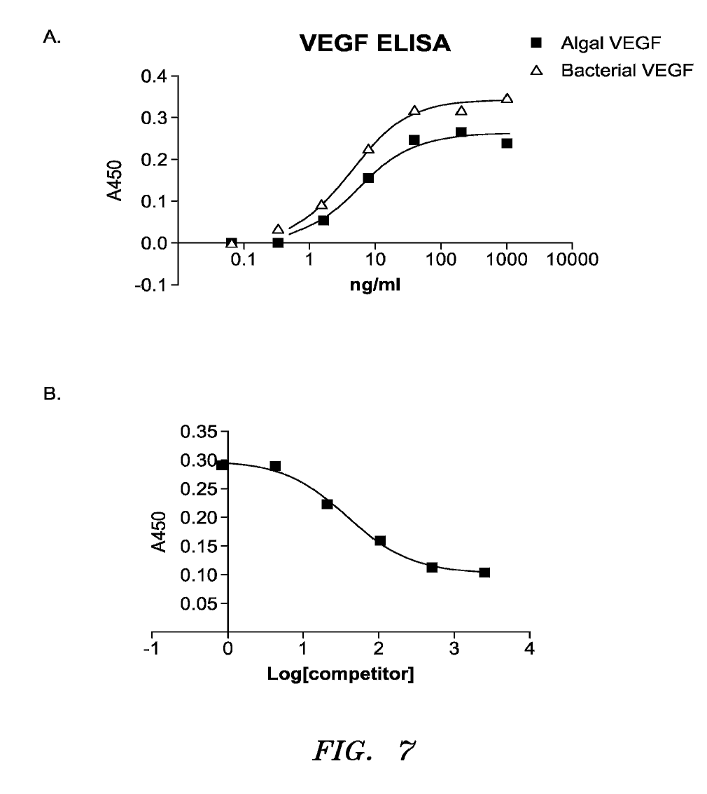

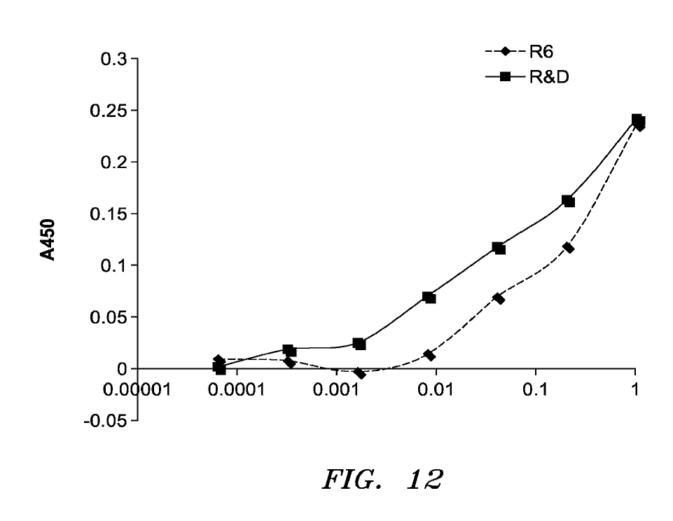

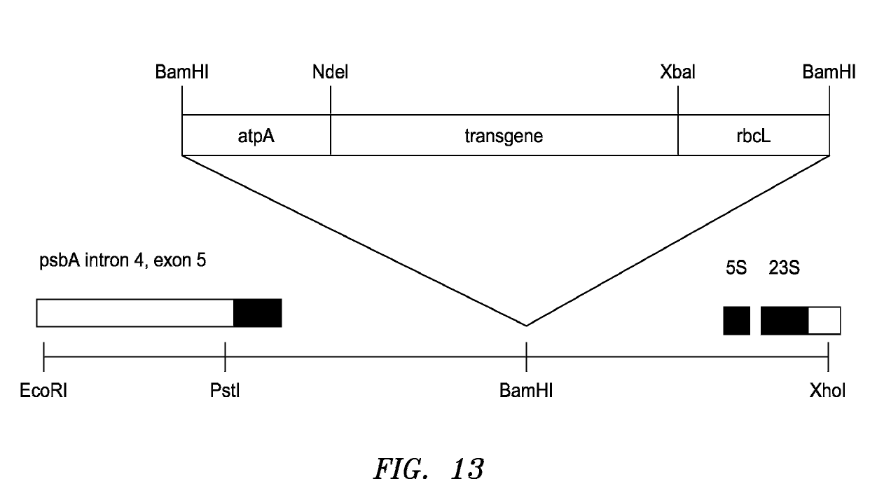

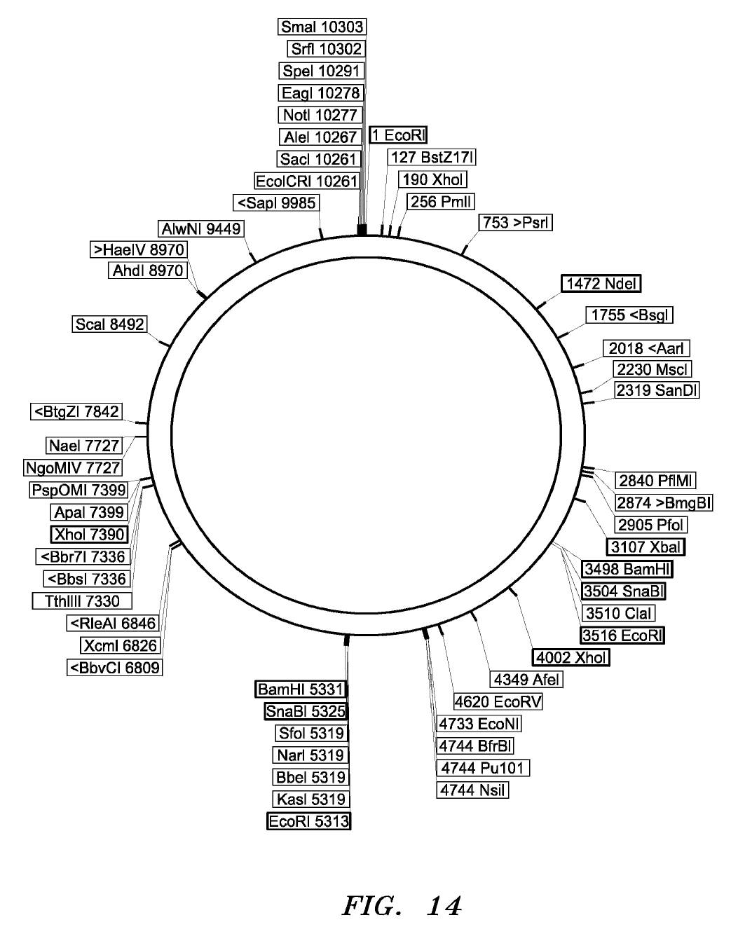

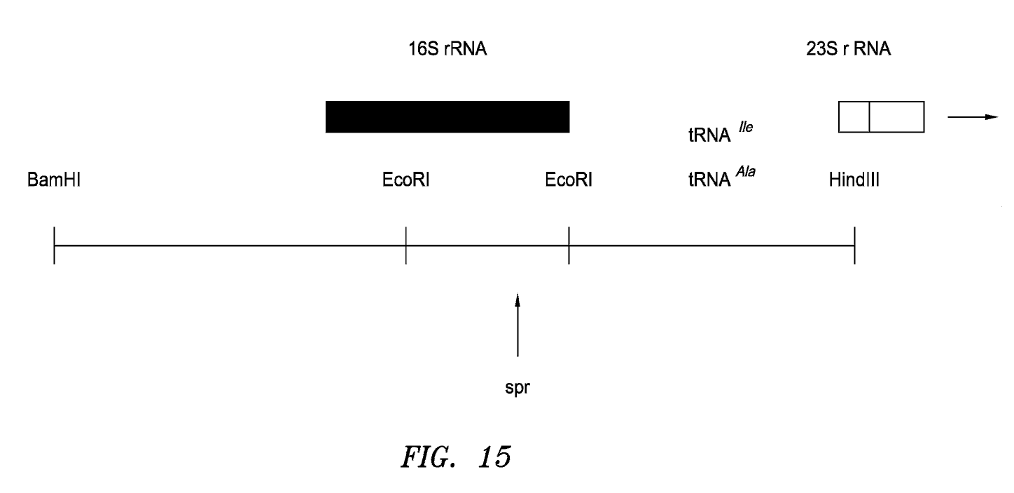

To test for bioactivity, purified VEGF and HMGB1 were assayed using a VEGF receptor-binding assay and a fibroblast chemotaxis assay, respectively. As potential antibody mimics, there is no bioactivity assay for 10FN3 or 14FN3. The most important characteristic of these recombinant proteins is that they are soluble, which we have shown to be the case when expressed in the chloroplast (FIG. 3 and FIGS. 6A-C). To determine whether algal-produced VEGF is biologically active, a sandwich ELISA was first performed to demonstrate antigenic integrity, an indicator of correct folding, and was used to establish effective concentration by reference to commercially available VEGF produced in E. coli (FIG. 7A). Algal-expressed VEGF was then compared to bacterial-expressed VEGF in a VEGF-receptor binding assay. Algal-expressed VEGF exhibited dose-dependent binding to the VEGF receptor, albeit with slightly lower affinity as compared to the control bacterial-expressed VEGF (FIG. 12; R6 is algal-expressed VEGF). This may be due to the presence of a small proportion of misfolded or truncated VEGF in the protein preparation. To determine VEGF bioactivity, a VEGF-receptor binding competition assay was used. VEGF derived from bacteria was able to compete with VEGF derived from algae for VEGF receptor binding (FIG. 7B). Bacterial VEGF displaced algal VEGF (200 ng/ml) from VEGFR with an IC50 of ˜40 ng/ml, consistent with a shared binding-site and broadly similar affinity. Overall, the data demonstrates that algal chloroplasts have the capability to express bioactive VEGF.

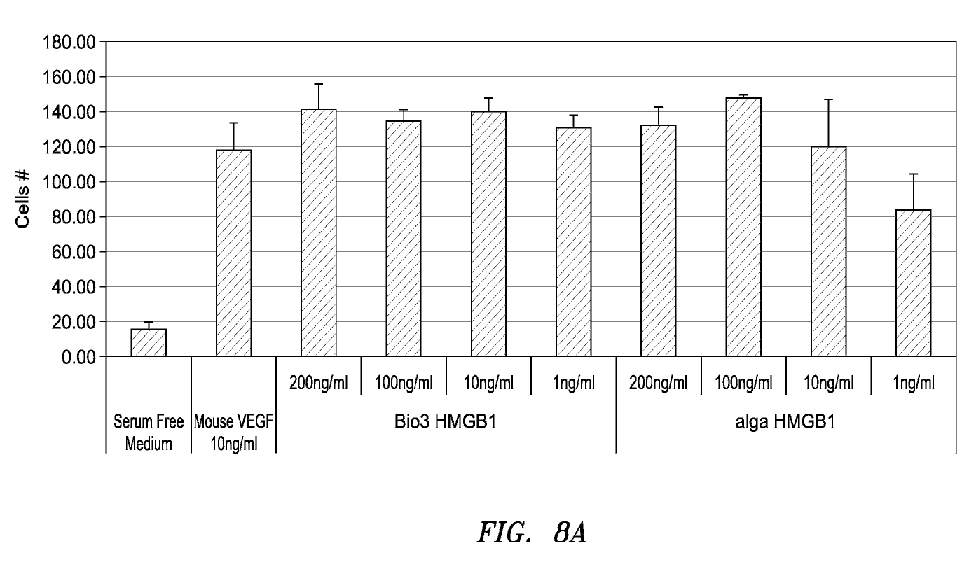

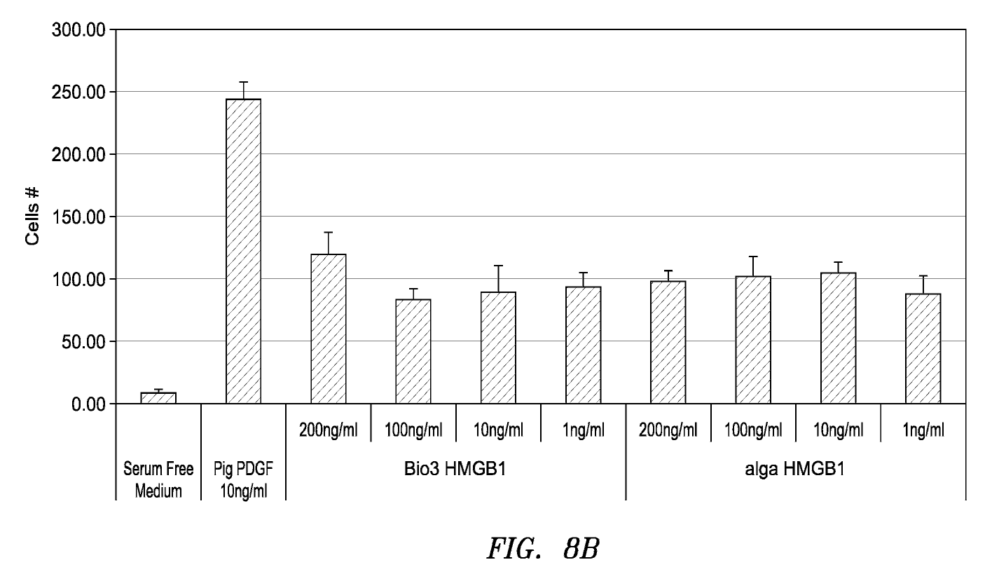

To determine whether algal-produced HMGB1 is biologically active, purified HMGB1 (˜1 mg) was sent to BioQuant (San Diego, Calif.), an independent contract research organization for bioactivity analysis using a mouse (A) or pig (B) fibroblast chemotaxis assay (FIGS. 8A and 8B). The algal expressed HMGB1 showed similar bioactivity to commercial bacterial-expressed HMGB1 (Bio3 HMGB1; HMGBiotech, Italy).

Taken together, these data indicate that high quantities of highly purified and bioactive human therapeutic proteins can be expressed in and purified from the chloroplast of C. reinhardtii.

### Example 12

### Activity Assays

VEGF Activity Assay

VEGF concentration was determined by ELISA. Maxisorp plates were coated with monoclonal anti-human VEGF (R&D Systems, Minneapolis, MIiN). After blocking with BSA, serial dilutions of VEGF purified from algae and commercial bacteria-derived VEGF (R&D Systems) were applied. After washing, bound VEGF was detected using a biotinylated polyclonal anti-human VEGF antibody (R&D Systems), alkaline-phosphatase-conjugated streptavidin and pNPP substrate (Sigma). Readings from uncoated wells were subtracted to give specific binding. (See FIG. 12.)

Binding of VEGF to receptor was assessed in a similar way, by coating Maxisorp plates with a human VEGF-R2(KDR):Fc fusion protein (R&D Systems), applying VEGF and detecting bound protein using biotinylated anti-VEGF. (See FIG. 7A.) For competition assays, a serial dilution of bacteria-derived VEGF was applied along with a constant concentration of algae-derived VEGF. Bound algae-derived VEGF was detected using HRP-conjugated anti-FLAG antibody (Sigma). (See FIG. 7B.)

HMGB1 Activity Assay

HMGB1 bioactivity was assessed using an in vitro analysis of the chemotactic effect of algal-expressed HMGB1. The relative ability of mouse and pig fibroblasts to migrate toward NIH3T3 conditioned media complemented with 10 ng/ml VEGF, PDGF or HMGB1 was assessed using a modified Boyden chamber (NeuroProbe, Inc., Gaithersburg, Md.). The cells were placed in the apical chambers of the apparatus and the media containing the chemotactic factors were placed in the basal chambers. A PVP membrane with 8 micron pores coated with 1 mg/ml collagen IV separated the apical and basal chambers. Cells were incubated for 24 hours at 37° C. The cells that migrated onto the basal surface of the membrane were stained and quantified using a microscope.

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-206. (canceled)

207. An isolated photosynthetic organism transformed with a polynucleotide comprising a first nucleotide sequence encoding a therapeutic protein, wherein the therapeutic protein is fibronectin domain 14 (14FN3), fibronectin domain 10 (10FN3), high-mobility group box 1 (HMGB1) protein, interferon beta, proinsulin, or vascular endothelial growth factor (VEGF), and wherein the photosynthetic organism is capable of expressing the therapeutic protein.

208. The organism of claim 207, wherein the organism is a cyanobacteria.

209. The organism of claim 207, wherein the organism is an alga.

210. The organism of claim 209, wherein the alga is Chlamydomonas reinhardtii.

211. The organism of claim 207, wherein the first nucleotide sequence encoding the therapeutic protein is codon-optimized to match the codon usage in a chloroplast of the organism.

212. The organism of claim 207, wherein the polynucleotide further comprises a second nucleotide sequence encoding a fusion protein, and the second nucleotide sequence is fused to the 5′ end of the first nucleotide sequence encoding the therapeutic protein.

213. The organism of claim 212, wherein the fusion protein is mammary-associated serum amyloid (M-SAA).

214. The organism of claim 213, wherein the polynucleotide further comprises a third nucleotide sequence encoding a proteolytic cleavage site between the second nucleotide sequence encoding the fusion protein and the first nucleotide sequence encoding the therapeutic protein.

215. The organism of claim 207, wherein the first nucleotide sequence comprises a nucleic acid sequence of SEQ ID NO: 83, SEQ ID NO: 82, SEQ ID NO: 90, SEQ ID NO: 84, SEQ ID NO: 86, or SEQ ID NO: 88.

216. The organism of claim 207, wherein the first nucleotide sequence comprises a nucleic acid sequence that has about 80% homology, about 85% homology, about 90% homology, about 95% homology, or about 99% homology to a nucleic acid sequence of SEQ ID NO: 83, SEQ ID NO: 82, SEQ ID NO: 90, SEQ ID NO: 84, SEQ ID NO: 86, or SEQ ID NO: 88, and wherein the therapeutic protein is biologically active.

217. The organism of claim 207, wherein the therapeutic protein is expressed at at least 0.5%, at least 1%, at least 1.5%, at least 2.0%, at least 2.5%, or at least 3.0% of total soluble protein.

218. A method of expressing a therapeutic protein in a photosynthetic organism, comprising:
transforming the photosynthetic organism with a polynucleotide comprising a first nucleotide sequence encoding the therapeutic protein, wherein the therapeutic protein is fibronectin domain 14 (14FN3), fibronectin domain 10 (10FN3), high-mobility group box 1 (HMGB1) protein, interferon beta, proinsulin, or vascular endothelial growth factor (VEGF), and expressing the therapeutic protein.

219. The method of claim 218, wherein the organism is a cyanobacteria.

220. The method of claim 218, wherein the organism is an alga.

221. The method of claim 218, wherein the first nucleotide sequence encoding the therapeutic protein is codon-optimized to match the codon usage in a chloroplast of the organism.

222. The method of claim 218, wherein the first nucleotide sequence comprises a nucleic acid sequence of SEQ ID NO: 83, SEQ ID NO: 82, SEQ ID NO: 90, SEQ ID NO: 84, SEQ ID NO: 86, or SEQ ID NO: 88.

223. The method of claim 218, wherein the first nucleotide sequence comprises a nucleic acid sequence that has about 80% homology, about 85% homology, about 90% homology, about 95% homology, or about 99% homology to a nucleic acid sequence of SEQ ID NO: 83, SEQ ID NO: 82, SEQ ID NO: 90, SEQ ID NO: 84, SEQ ID NO: 86, or SEQ ID NO: 88, and wherein the therapeutic protein is biologically active.

224. The method of claim 218, wherein the transformation is by particle bombardment.

225. The method of claim 218, wherein the therapeutic protein is expressed at at least 0.5%, at least 1%, at least 1.5%, at least 2.0%, at least 2.5%, or at least 3.0% of total soluble protein.

226. A therapeutic protein made by the method of claim 218.

227. The organism of claim 207, wherein a chloroplast of said organism is transformed with said polynucleotide.

228. The organism of claim 227, wherein said organism is homoplasmic.

229. The organism of claim 207, wherein the polynucleotide further comprises a second nucleotide sequence encoding a fusion protein, and the second nucleotide sequence is fused to the 3′ end of the first nucleotide sequence encoding the therapeutic protein.


SEQUENCE LISTING (92 sequences)

SEQ ID NO: 1 - DNA, 600 bp, Artificial Sequence
  codon-optimized EPO sequence
catatggtaccagctccacctcgtttaatttgtgactctcgtgtattagaacgttatttattagaagcaaaagaggcagaaaatattactactggttgtgcagaacattgttcattaaatgaaaacattacagttccagatacaaaagttaatttttacgcttggaaacgtatggaagtaggacaacaagcagtagaagtatggcaaggtttagctttattatcagaagcagttttaagaggtcaagcattattagtaaattcatcacaaccttgggaaccattacaattacatgttgataaagctgtttcaggtcttagatctttaactactttattacgtgctcttggagctcaaaaagaagctatttcacctccagacgctgcaagtgctgcacctcttcgtacaatcactgctgatacattccgtaaattatttcgtgtttactcaaattttcttcgtggtaaattaaaattatatactggtgaagcatgtcgtacaggtgatcgtggtaccggtgaaaacttatactttcaaggctcaggtggcggtggaagtgattacaaagatgatgatgataaaggaaccggttaatctaga

SEQ ID NO: 2 - protein, 196 aa, Artificial Sequence
  modified protein sequence
MVPAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGTGENLYFQGSGGGGSDYKDDDDKGTG

SEQ ID NO: 3 - DNA, 384 bp, Artificial Sequence
  codon-optimized 10FN3 sequence
catatggtaccagtaagtgatgttccacgtgatcttgaagtagtagcagcaactccaacttcattattaatttcatgggatgcacctgctgttacagtacgttattaccgtattacttatggtgagactggtggtaactctccagttcaagaatttactgttcctggttcaaaatcaacagcaacaatttcaggattaaaaccaggtgttgattatactattacagtatatgcagttacaggtcgtggtgattcaccagcttcatcaaaacctatttcaatcaattatcgtacaggtaccggtgaaaacttatactttcaaggctcaggtggcggtggaagtgattacaaagatgatgatgataaaggaaccggttaatctaga

SEQ ID NO: 4 - protein, 124 aa, Artificial Sequence
  modified protein sequence
MVPVSDVPRDLEVVAATPTSLLISWDAPAVTVRYYRITYGETGGNSPVQEFTVPGSKSTATISGLKPGVDYTITVYAVTGRGDSPASSKPISINYRTGTGENLYFQGSGGGGSDYKDDDDKGTG

SEQ ID NO: 5 - DNA, 369 bp, Artificial Sequence
  codon-optimized 14FN3 sequence
catatggtaccaaatgttagtcctcctcgtagagctagagttacagatgcaacagaaacaacaattacaatttcttggcgtacaaaaactgaaactatcactggttttcaagtagatgcagttccagcaaatggtcaaacacctattcaacgtacaatcaaaccagacgttagatcatatactattacaggtttacaaccaggtacagattataaaatttatttatatacattaaatgacaacgctcgtagttcacctgtagttattgatgcttcaactggtaccggtgaaaacttatactttcaaggctcaggtggcggtggaagtgattacaaagatgatgatgataaaggaaccggttaatctaga

SEQ ID NO: 6 - protein, 119 aa, Artificial Sequence
  modified protein sequence
MVPNVSPPRRARVTDATETTITISWRTKTETITGFQVDAVPANGQTPIQRTIKPDVRSYTITGLQPGTDYKIYLYTLNDNARSSPVVIDASTGTGENLYFQGSGGGGSDYKDDDDKGTG

SEQ ID NO: 7 - DNA, 597 bp, Artificial Sequence
  codon-optimized interferon beta sequence
catatggtaccatcttataatttattaggatttttacaacgtagttctaactttcaatgtcaaaaattattatggcaattaaatggtcgtttagaatactgcttaaaagaccgtatgaattttgatattccagaagaaattaaacaattacaacaatttcaaaaagaggatgctgctttaacaatttatgaaatgttacaaaacatttttgctatttttcgtcaagattcatcatcaacaggttggaacgaaactattgttgaaaaccttttagcaaatgtttatcaccaaatcaatcacttaaaaacagtattagaagaaaaattagaaaaagaagattttacaagaggtaaattaatgtcatcattacatttaaaacgttattacggtcgtattttacattatttaaaagctaaagaatattcacattgtgcttggacaattgttcgtgttgaaattcttcgtaatttctattttattaaccgtttaacaggatacttaagaaacggtaccggtgaaaacttatactttcaaggctcaggtggcggtggaagtgattacaaagatgatgatgataaaggaaccggttaatctaga

SEQ ID NO: 8 - protein, 195 aa, Artificial Sequence
  modified protein sequence
MVPSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGTGENLYFQGSGGGGSDYKDDDDKGTG

SEQ ID NO: 9 - DNA, 360 bp, Artificial Sequence
  codon-optimized proinsulin sequence
catatggtaccatttgtaaatcaacatttatgtggaagtcacttagttgaagcattatatttagtttgtggtgagcgtggtttcttttatacaccaaaaacacgtcgtgaagctgaagacttacaagttggtcaagttgagttaggaggaggacctggtgctggttctttacaacctttagctcttgaaggttcattacaaaaacgtggtattgttgaacaatgttgcacaagtatttgtagtttatatcaattagaaaattattgtaacggtaccggtgaaaacttatactttcaaggctcaggtggcggtggaagtgattacaaagatgatgatgataaaggaaccggttaatctaga

SEQ ID NO: 10 - protein, 116 aa, Artificial Sequence
  modified protein sequence
MVPFVNQHLCGSHLVEALYLVCGERGFFYTPKTRREAEDLQVGQVELGGGPGAGSLQPLALEGSLQKRGIVEQCCTSICSLYQLENYCNGTGENLYFQGSGGGGSDYKDDDDKGTG

SEQ ID NO: 11 - DNA, 465 bp, Artificial Sequence
  codon-optimized VEGF sequence
catatggtaccagctccaatggctgaaggtggaggtcaaaaccaccacgaagtagtaaaatttatggacgtataccaaagatcatactgtcacccaattgaaactttagtagatatttttcaagaataccctgatgaaattgaatatatctttaaaccaagttgtgttcctcttatgcgttgtggtggatgttgtaacgatgaaggattagaatgtgtacctacagaagagtcaaatattactatgcaaattatgagaatcaaaccacatcaaggtcaacacattggtgaaatgagtttccttcaacataataaatgtgaatgtcgtccaaaaaaagatcgtgctagacaagaaaattgtgataaacctcgtcgtggtaccggtgaaaacttatactttcaaggctcaggtggcggtggaagtgattacaaagatgatgatgataaaggaaccggttaatctaga

SEQ ID NO: 12 - protein, 151 aa, Artificial Sequence
  modified protein sequence
MVPAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENCDKPRRGTGENLYFQGSGGGGSDYKDDDDKGTG

SEQ ID NO: 13 - DNA, 654 bp, Artificial Sequence
  codon-optimized HMGB1 sequence
catatggtaccaggtaaaggagatccaaaaaaacctcgtggtaaaatgagttcatacgcttttttcgtacaaacatgccgtgaagaacacaaaaagaaacatcctgatgcttcagttaatttttctgaattttctaaaaaatgttcagaacgttggaaaacaatgagtgctaaagaaaaaggtaaattcgaagacatggctaaagcagacaaagctcgttatgaacgtgaaatgaaaacttatattcctcctaaaggcgaaacaaagaaaaaatttaaagatccaaatgctccaaaacgtccaccaagtgcttttttcttattttgttcagaatatcgtccaaaaattaaaggtgaacacccaggtttatctattggtgatgttgctaaaaaattaggtgaaatgtggaacaatacagctgctgacgataaacaaccttatgaaaaaaaagctgctaaattaaaagagaaatacgaaaaagatattgctgcttatagagctaaaggtaaacctgatgctgcaaaaaaaggtgtagtaaaagctgaaaaatcaaaaaagaaaaaaggtaccggtgaaaacttatactttcaaggctcaggtggcggtggaagtgattacaaagatgatgatgataaaggaaccggttaatctaga

SEQ ID NO: 14 - protein, 214 aa, Artificial Sequence
  modified protein sequence
MVPGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKGTGENLYFQGSGGGGSDYKDDDDKGTG

SEQ ID NO: 15 - DNA, 591 bp, Artificial Sequence
  codon-optimized erythropoietin sequence
atggtaccagctccacctcgtttaatttgtgactctcgtgtattagaacgttatttattagaagcaaaagaggcagaaaatattactactggttgtgcagaacattgttcattaaatgaaaacattacagttccagatacaaaagttaatttttacgcttggaaacgtatggaagtaggacaacaagcagtagaagtatggcaaggtttagctttattatcagaagcagttttaagaggtcaagcattattagtaaattcatcacaaccttgggaaccattacaattacatgttgataaagctgtttcaggtcttagatctttaactactttattacgtgctcttggagctcaaaaagaagctatttcacctccagacgctgcaagtgctgcacctcttcgtacaatcactgctgatacattccgtaaattatttcgtgtttactcaaattttcttcgtggtaaattaaaattatatactggtgaagcatgtcgtacaggtgatcgtggtaccggtgaaaacttatactttcaaggctcaggtggcggtggaagtgattacaaagatgatgatgataaaggaaccggttaa

SEQ ID NO: 16 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 17 - DNA, 375 bp, Artificial Sequence
  codon-optimized 10FN3 sequence
atggtaccagtaagtgatgttccacgtgatcttgaagtagtagcagcaactccaacttcattattaatttcatgggatgcacctgctgttacagtacgttattaccgtattacttatggtgagactggtggtaactctccagttcaagaatttactgttcctggttcaaaatcaacagcaacaatttcaggattaaaaccaggtgttgattatactattacagtatatgcagttacaggtcgtggtgattcaccagcttcatcaaaacctatttcaatcaattatcgtacaggtaccggtgaaaacttatactttcaaggctcaggtggcggtggaagtgattacaaagatgatgatgataaaggaaccggttaa

SEQ ID NO: 18 - protein, 94 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 19 - DNA, 360 bp, Artificial Sequence
  codon-optimized 14FN3 sequence
atggtaccaaatgttagtcctcctcgtagagctagagttacagatgcaacagaaacaacaattacaatttcttggcgtacaaaaactgaaactatcactggttttcaagtagatgcagttccagcaaatggtcaaacacctattcaacgtacaatcaaaccagacgttagatcatatactattacaggtttacaaccaggtacagattataaaatttatttatatacattaaatgacaacgctcgtagttcacctgtagttattgatgcttcaactggtaccggtgaaaacttatactttcaaggctcaggtggcggtggaagtgattacaaagatgatgatgataaaggaaccggttaa

SEQ ID NO: 20 - protein, 89 aa, Homo sapiens
NVSPPRRARVTDATETTITISWRTKTETITGFQVDAVPANGQTPIQRTIKPDVRSYTITGLQPGTDYKIYLYTLNDNARSSPVVIDAST

SEQ ID NO: 21 - DNA, 588 bp, Artificial Sequence
  codon-optimized interferon beta sequence
atggtaccatcttataatttattaggatttttacaacgtagttctaactttcaatgtcaaaaattattatggcaattaaatggtcgtttagaatactgcttaaaagaccgtatgaattttgatattccagaagaaattaaacaattacaacaatttcaaaaagaggatgctgctttaacaatttatgaaatgttacaaaacatttttgctatttttcgtcaagattcatcatcaacaggttggaacgaaactattgttgaaaaccttttagcaaatgtttatcaccaaatcaatcacttaaaaacagtattagaagaaaaattagaaaaagaagattttacaagaggtaaattaatgtcatcattacatttaaaacgttattacggtcgtattttacattatttaaaagctaaagaatattcacattgtgcttggacaattgttcgtgttgaaattcttcgtaatttctattttattaaccgtttaacaggatacttaagaaacggtaccggtgaaaacttatactttcaaggctcaggtggcggtggaagtgattacaaagatgatgatgataaaggaaccggttaa

SEQ ID NO: 22 - protein, 187 aa, Homo sapiens [sequence omitted]

SEQ ID NO: 23 - DNA, 351 bp, Artificial Sequence
  codon-optimized proinsulin sequence
atggtaccatttgtaaatcaacatttatgtggaagtcacttagttgaagcattatatttagtttgtggtgagcgtggtttcttttatacaccaaaaacacgtcgtgaagctgaagacttacaagttggtcaagttgagttaggaggaggacctggtgctggttctttacaacctttagctcttgaaggttcattacaaaaacgtggtattgttgaacaatgttgcacaagtatttgtagtttatatcaattagaaaattattgtaacggtaccggtgaaaacttatactttcaaggctcaggtggcggtggaagtgattacaaagatgatgatgataaaggaaccggttaa

SEQ ID NO: 24 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 25 - DNA, 456 bp, Artificial Sequence
  codon-optimized VEGF sequence
atggtaccagctccaatggctgaaggtggaggtcaaaaccaccacgaagtagtaaaatttatggacgtataccaaagatcatactgtcacccaattgaaactttagtagatatttttcaagaataccctgatgaaattgaatatatctttaaaccaagttgtgttcctcttatgcgttgtggtggatgttgtaacgatgaaggattagaatgtgtacctacagaagagtcaaatattactatgcaaattatgagaatcaaaccacatcaaggtcaacacattggtgaaatgagtttccttcaacataataaatgtgaatgtcgtccaaaaaaagatcgtgctagacaagaaaattgtgataaacctcgtcgtggtaccggtgaaaacttatactttcaaggctcaggtggcggtggaagtgattacaaagatgatgatgataaaggaaccggttaa

SEQ ID NO: 26 - protein, 147 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 27 - DNA, 645 bp, Artificial Sequence
  codon-optimized HMGB1 sequence
atggtaccaggtaaaggagatccaaaaaaacctcgtggtaaaatgagttcatacgcttttttcgtacaaacatgccgtgaagaacacaaaaagaaacatcctgatgcttcagttaatttttctgaattttctaaaaaatgttcagaacgttggaaaacaatgagtgctaaagaaaaaggtaaattcgaagacatggctaaagcagacaaagctcgttatgaacgtgaaatgaaaacttatattcctcctaaaggcgaaacaaagaaaaaatttaaagatccaaatgctccaaaacgtccaccaagtgcttttttcttattttgttcagaatatcgtccaaaaattaaaggtgaacacccaggtttatctattggtgatgttgctaaaaaattaggtgaaatgtggaacaatacagctgctgacgataaacaaccttatgaaaaaaaagctgctaaattaaaagagaaatacgaaaaagatattgctgcttatagagctaaaggtaaacctgatgctgcaaaaaaaggtgtagtaaaagctgaaaaatcaaaaaagaaaaaaggtaccggtgaaaacttatactttcaaggctcaggtggcggtggaagtgattacaaagatgatgatgataaaggaaccggttaa

SEQ ID NO: 28 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 29 - DNA, 26 bp, Artificial Sequence
  PCR Primer
ggaaggggaggacgtaggtacataaa

SEQ ID NO: 30 - DNA, 23 bp, Artificial Sequence
  PCR Primer
ttagaacgtgttttgttcccaat

SEQ ID NO: 31 - DNA, 21 bp, Artificial Sequence
  PCR Primer
cccataaatagtttcaattgg

SEQ ID NO: 32 - DNA, 27 bp, Artificial Sequence
  PCR Primer
cggtggttattccaggccaaacttatg

SEQ ID NO: 33 - DNA, 20 bp, Artificial Sequence
  PCR Primer
ccgaactgaggttgggttta

SEQ ID NO: 34 - DNA, 20 bp, Artificial Sequence
  PCR Primer
gggggagcgaataggattag

SEQ ID NO: 35 - DNA, 27 bp, Artificial Sequence
  PCR Primer for psbA promoter
gtgctaggtaactaacgtttgattttt

SEQ ID NO: 36 - DNA, 22 bp, Artificial Sequence
  PCR Primer for atpA promoter
caagggtgaaccattacttttg

SEQ ID NO: 37 - DNA, 20 bp, Artificial Sequence
  PCR Primer for EPO
ggttcccaaggttgtgatga

SEQ ID NO: 38 - DNA, 24 bp, Artificial Sequence
  PCR Primer for 10FN3
ggttttgatgaagctgctggtgaa

SEQ ID NO: 39 - DNA, 20 bp, Artificial Sequence
  PCR Primer for 14FN3
accggtaccagttgaagcat

SEQ ID NO: 40 - DNA, 19 bp, Artificial Sequence
  PCR Primer for interferon beta
cgttccaccctgttgatga

SEQ ID NO: 41 - DNA, 20 bp, Artificial Sequence
  PCR Primer for proinsulin
ccggtaccgttacaataatc

SEQ ID NO: 42 - DNA, 21 bp, Artificial Sequence
  PCR Primer for VEGF
tgttgaccttgatgtggtttg

SEQ ID NO: 43 - DNA, 22 bp, Artificial Sequence
  PCR Primer for HMGB1
gcatttggatctttaaattttt

SEQ ID NO: 44 - DNA, 20 bp, Artificial Sequence
  Forward PCR Primer for EPO
tacgcttggaaacgtatgga

SEQ ID NO: 45 - DNA, 20 bp, Artificial Sequence
  Reverse PCR Primer for EPO
tgagctccaagagcacgtaa

SEQ ID NO: 46 - DNA, 20 bp, Artificial Sequence
  Forward PCR Primer for 10FN3
aatttcatgggatgcacctg

SEQ ID NO: 47 - DNA, 20 bp, Artificial Sequence
  Reverse PCR Primer for 10FN3
tcaccacgacctgtaactgc

SEQ ID NO: 48 - DNA, 21 bp, Artificial Sequence
  Forward PCR Primer for 14FN3
ccaaatgttagtcctcctcgt

SEQ ID NO: 49 - DNA, 22 bp, Artificial Sequence
  Reverse PCR Primer for 14FN3
cgtctggtttgattgtacgttg

SEQ ID NO: 50 - DNA, 22 bp, Artificial Sequence
  Forward PCR Primer for interferon beta
ttatggcaattaaatggtcgtt

SEQ ID NO: 51 - DNA, 20 bp, Artificial Sequence
  Reverse PCR Primer for interferon beta
tcgttccaacctgttgatga

SEQ ID NO: 52 - DNA, 20 bp, Artificial Sequence
  Forward PCR Primer for proinsulin
acacgtcgtgaagctgaaga

SEQ ID NO: 53 - DNA, 20 bp, Artificial Sequence
  Reverse PCR Primer for proinsulin
ttcaccggtaccgttacaat

SEQ ID NO: 54 - DNA, 20 bp, Artificial Sequence
  Forward PCR Primer for VEGF
aggtcaaaaccaccacgaag

SEQ ID NO: 55 - DNA, 20 bp, Artificial Sequence
  Reverse PCR Primer for VEGF
catccaccacaacgcataag

SEQ ID NO: 56 - DNA, 20 bp, Artificial Sequence
  Forward PCR Primer for HMGB1
cgttggaaaacaatgagtgc

SEQ ID NO: 57 - DNA, 20 bp, Artificial Sequence
  Reverse PCR Primer for HMGB1
acttggtggacgttttggag

SEQ ID NO: 58 - DNA, 20 bp, Artificial Sequence
  Forward PCR Primer for Rbcl
agcaggtgctggattcaaag

SEQ ID NO: 59 - DNA, 20 bp, Artificial Sequence
  Reverse PCR Primer for Rbcl
cagctacagcagcaccacat

SEQ ID NO: 60 - protein, 8 aa, Artificial Sequence
  TAG
DYKDDDDK

SEQ ID NO: 61 - protein, 6 aa, Artificial Sequence
  LINKER
SGGGGS

SEQ ID NO: 62 - protein, 7 aa, Artificial Sequence
  TAG
ENLYFQG

SEQ ID NO: 63 - DNA, 544 bp, Artificial Sequence
  modified atpA promoter/5′ UTR
ggatcccatttttataactggtctcaaaatacctataaacccattgttcttctcttttagctctaagaacaatcaatttataaatatatttattattatgctataatataaatactatataaatacatttacctttttataaatacatttaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttcttttttcatatg

SEQ ID NO: 64 - DNA, 18 bp, Artificial Sequence
  LINKER
tcaggtggcggtggaagt

SEQ ID NO: 65 - DNA, 432 bp, Artificial Sequence
  modified psbD promoter/5′ UTR
ggatccatgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcatatg

SEQ ID NO: 66 - DNA, 392 bp, Artificial Sequence
  modified psbA 3′ UTR
tctagacttagcttcaactaactctagctcaaacaactaatttttttttaaactaaaataaatctggttaaccatacctggtttattttagtttatacacacttttcatatatatatacttaatagctaccataggcagttggcaggacgtccccttacgggacaaatgtatttattgttgcctgccaactgcctaatataaatattagtggacgtccccttccccttacgggcaagtaaacttagggattttaatgctccgttaggaggcaaataaattttagtggcagttgcctcgcctatcggctaacaagttccttcggagtatataaatatcctgccaactgccgatatttatatactaggcagtggcggtaccactcgacggatcc

SEQ ID NO: 67 - DNA, 435 bp, Artificial Sequence
  modified rbcL 3′ UTR
tctagagtcgacctgcaggcatgcaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaatttttatttttcatgatgtttatgtgaatagcataaacatcgtttttattttttatggtgtttaggttaaatacctaaacatcattttacatttttaaaattaagttctaaagttatcttttgtttaaatttgcctgtgctttataaattacgatgtgccagaaaaataaaatcttagctttttattatagaatttatctttatgtattatattttataagtaataaaagaaatagtaacatacgtcgacggatcc

SEQ ID NO: 68 - DNA, 582 bp, Homo sapiens
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 69 - DNA, 282 bp, Homo sapiens
gtttctgatgttccgagggacctggaagttgttgctgcgacccccaccagcctactgatcagctgggatgctcctgctgtcacagtgagatattacaggatcacttacggagagacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaaca

SEQ ID NO: 70 - DNA, 267 bp, Homo sapiens
aatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccact

SEQ ID NO: 71 - DNA, 564 bp, Homo sapiens
atgaccaacaagtgtctcctccaaattgctctcctgttgtgcttctccacgacagctctttccatgagctacaacttgcttggattcctacaaagaagcagcaattgtcagtgtcagaagctcctgtggcaattgaatgggaggcttgaatactgcctcaaggacaggaggaactttgacatccctgaggagattaagcagctgcagcagttccagaaggaggacgccgcagtgaccatctatgagatgctccagaacatctttgctattttcagacaagattcatcgagcactggctggaatgagactattgttgagaacctcctggctaatgtctatcatcagagaaaccatctgaagacagtcctggaagaaaaactggagaaagaagatttcaccaggggaaaacgcatgagcagtctgcacctgaaaagatattatgggaggattctgcattacctgaaggccaaggaggacagtcactgtgcctggaccatagtcagagtggaaatcctaaggaacttttacgtcattaacagacttacaggttacctccgaaactga

SEQ ID NO: 72 - DNA, 333 bp, Homo sapiens
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 73 - DNA, 444 bp, Homo sapiens
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggtga

SEQ ID NO: 74 - DNA, 648 bp, Homo sapiens
atgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataa

SEQ ID NO: 75 - DNA, 780 bp, Artificial Sequence
  codon-optimized aphA6 gene
atggaattgcccaatattattcaacaatttatcggaaacagcgttttagagccaaataaaattggtcagtcgccatcggatgtttattcttttaatcgaaataatgaaactttttttcttaagcgatctagcactttatatacagagaccacatacagtgtctctcgtgaagcgaaaatgttgagttggctctctgagaaattaaaggtgcctgaactcatcatgacttttcaggatgagcagtttgaatttatgatcactaaagcgatcaatgcaaaaccaatttcagcgctttttttaacagaccaagaattgcttgctatctataaggaggcactcaatctgttaaattcaattgctattattgattgtccatttatttcaaacattgatcatcggttaaaagagtcaaaattttttattgataaccaactccttgacgatatagatcaagatgattttgacactgaattatggggagaccataaaacttacctaagtctatggaatgagttaaccgagactcgtgttgaagaaagattggttttttctcatggcgatatcacggatagtaatatttttatagataaattcaatgaaatttattttttagaccttggtcgtgctgggttagcagatgaatttgtagatatatcctttgttgaacgttgcctaagagaggatgcatcggaggaaactgcgaaaatatttttaaagcatttaaaaaatgatagacctgacaaaaggaattattttttaaaacttgatgaattgaattga

SEQ ID NO: 76 - protein, 259 aa, Chlamydomonas reinhardtii
MELPNIIQQFIGNSVLEPNKIGQSPSDVYSFNRNNETFFLKRSSTLYTETTYSVSREAKMLSWLSEKLKVPELIMTFQDEQFEFMITKAINAKPISALFLTDQELLAIYKEALNLLNSIAIIDCPFISNIDHRLKESKFFIDNQLLDDIDQDDFDTELWGDHKTYLSLWNELTETRVEERLVFSHGDITDSNIFIDKFNEIYFLDLGRAGLADEFVDISFVERCLREDASEETAKIFLKHLKNDRPDKRNYFLKLDELN

SEQ ID NO: 77 - protein, 215 aa, Mus musculus
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEDDEEDEEDEEEEEEEEDEDEEEDDDDE

SEQ ID NO: 78 - protein, 215 aa, Rattus norvegicus; identical to SEQ ID NO: 77

SEQ ID NO: 79 - protein, 215 aa, Canis lupus familiaris; identical to SEQ ID NO: 28

SEQ ID NO: 80 - DNA, 498 bp, Artificial Sequence
  codon-optimized EPO sequence
gctccacctcgtttaatttgtgactctcgtgtattagaacgttatttattagaagcaaaagaggcagaaaatattactactggttgtgcagaacattgttcattaaatgaaaacattacagttccagatacaaaagttaatttttacgcttggaaacgtatggaagtaggacaacaagcagtagaagtatggcaaggtttagctttattatcagaagcagttttaagaggtcaagcattattagtaaattcatcacaaccttgggaaccattacaattacatgttgataaagctgtttcaggtcttagatctttaactactttattacgtgctcttggagctcaaaaagaagctatttcacctccagacgctgcaagtgctgcacctcttcgtacaatcactgctgatacattccgtaaattatttcgtgtttactcaaattttcttcgtggtaaattaaaattatatactggtgaagcatgtcgtacaggtgatcgt

SEQ ID NO: 81 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 82 - DNA, 282 bp, Artificial Sequence
  codon-optimized 10FN3 sequence
gtaagtgatgttccacgtgatcttgaagtagtagcagcaactccaacttcattattaatttcatgggatgcacctgctgttacagtacgttattaccgtattacttatggtgagactggtggtaactctccagttcaagaatttactgttcctggttcaaaatcaacagcaacaatttcaggattaaaaccaggtgttgattatactattacagtatatgcagttacaggtcgtggtgattcaccagcttcatcaaaacctatttcaatcaattatcgtaca

SEQ ID NO: 83 - DNA, 267 bp, Artificial Sequence
  codon-optimized 14FN3 sequence
aatgttagtcctcctcgtagagctagagttacagatgcaacagaaacaacaattacaatttcttggcgtacaaaaactgaaactatcactggttttcaagtagatgcagttccagcaaatggtcaaacacctattcaacgtacaatcaaaccagacgttagatcatatactattacaggtttacaaccaggtacagattataaaatttatttatatacattaaatgacaacgctcgtagttcacctgtagttattgatgcttcaact

SEQ ID NO: 84 - DNA, 495 bp, Artificial Sequence
  codon-optimized interferon beta sequence
tcttataatttattaggatttttacaacgtagttctaactttcaatgtcaaaaattattatggcaattaaatggtcgtttagaatactgcttaaaagaccgtatgaattttgatattccagaagaaattaaacaattacaacaatttcaaaaagaggatgctgctttaacaatttatgaaatgttacaaaacatttttgctatttttcgtcaagattcatcatcaacaggttggaacgaaactattgttgaaaaccttttagcaaatgtttatcaccaaatcaatcacttaaaaacagtattagaagaaaaattagaaaaagaagattttacaagaggtaaattaatgtcatcattacatttaaaacgttattacggtcgtattttacattatttaaaagctaaagaatattcacattgtgcttggacaattgttcgtgttgaaattcttcgtaatttctattttattaaccgtttaacaggatacttaagaaac

SEQ ID NO: 85 - protein, 165 aa, Homo sapiens
SYNLLGFLQRSSNCQCQKLLWQLNGRLEYCLKDRRNFDIPEEIKQLQQFQKEDAAVTIYEMLQNIFAIFRQDSSSTGWNETIVENLLANVYHQRNHLKTVLEEKLEKEDFTRGKRMSSLHLKRYYGRILHYLKAKEDSHCAWTIVRVEILRNFYVINRLTGYLRN

SEQ ID NO: 86 - DNA, 258 bp, Artificial Sequence
  codon-optimized proinsulin sequence
tttgtaaatcaacatttatgtggaagtcacttagttgaagcattatatttagtttgtggtgagcgtggtttcttttatacaccaaaaacacgtcgtgaagctgaagacttacaagttggtcaagttgagttaggaggaggacctggtgctggttctttacaacctttagctcttgaaggttcattacaaaaacgtggtattgttgaacaatgttgcacaagtatttgtagtttatatcaattagaaaattattgtaac

SEQ ID NO: 87 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 88 - DNA, 363 bp, Artificial Sequence
  codon-optimized VEGF sequence
gctccaatggctgaaggtggaggtcaaaaccaccacgaagtagtaaaatttatggacgtataccaaagatcatactgtcacccaattgaaactttagtagatatttttcaagaataccctgatgaaattgaatatatctttaaaccaagttgtgttcctcttatgcgttgtggtggatgttgtaacgatgaaggattagaatgtgtacctacagaagagtcaaatattactatgcaaattatgagaatcaaaccacatcaaggtcaacacattggtgaaatgagtttccttcaacataataaatgtgaatgtcgtccaaaaaaagatcgtgctagacaagaaaattgtgataaacctcgtcgt

SEQ ID NO: 89 - protein, 121 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 90 - DNA, 552 bp, Artificial Sequence
  codon-optimized HMGB1 sequence
ggtaaaggagatccaaaaaaacctcgtggtaaaatgagttcatacgcttttttcgtacaaacatgccgtgaagaacacaaaaagaaacatcctgatgcttcagttaatttttctgaattttctaaaaaatgttcagaacgttggaaaacaatgagtgctaaagaaaaaggtaaattcgaagacatggctaaagcagacaaagctcgttatgaacgtgaaatgaaaacttatattcctcctaaaggcgaaacaaagaaaaaatttaaagatccaaatgctccaaaacgtccaccaagtgcttttttcttattttgttcagaatatcgtccaaaaattaaaggtgaacacccaggtttatctattggtgatgttgctaaaaaattaggtgaaatgtggaacaatacagctgctgacgataaacaaccttatgaaaaaaaagctgctaaattaaaagagaaatacgaaaaagatattgctgcttatagagctaaaggtaaacctgatgctgcaaaaaaaggtgtagtaaaagctgaaaaatcaaaaaagaaaaaa

SEQ ID NO: 91 - protein, 184 aa, Homo sapiens
GKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKK

SEQ ID NO: 92 - DNA, 1477 bp, Artificial Sequence
  modified Chlamydomonas reinhardtii sequence
gaattccatatttagataaacgatttcaagcagcagaattagctttattagaacaaacttgtaaagaaatgaatgtaccaatgccgcgcattgtagaaaaaccagataattattatcaaattcgacgtatacgtgaattaaaacctgatttaacgattactggaatggcacatgcaaatccattagaagctcgaggtattacaacaaaatggtcagttgaatttacttttgctcaaattcatggatttactaatacacgtgaaattttagaattagtaacacagcctcttagacgcaatctaatgtcaaatcaatctgtaaatgctatttcttaatataaatcccaaaagattttttttataatactgagacttcaacacttacttgtttttattttttgtagttacaattcactcacgttaaagacattggaaaatgaggcaggacgttagtcgatatttatacactcttaagtttacttgcccaatatttatattaggacgtccccttcgggtaaataaattttagtggcagtggtaccaccactgcctattttaatactccgaagcatataaatatacttcggagtatataaatatccactaatatttatattaggcagttggcaggcaacaataaataaatttgtcccgtaaggggacgtcccgaaggggaaggggaagaaggcagttgcctcgcctatcggctaacaagttcctttggagtatataaccgcctacaggtaacttaaagaacatttgttacccgtaggggtttatacttctaattgcttcttctgaacaataaaatggtttgtgtggtctgggctaggaaacttgtaacaatgtgtagtgtcgcttccgcttcccttcgggacgtccccttcgggtaagtaaacttaggagtattaaatcgggacgtccccttcgggtaaataaatttcagtggacgtccccttacgggacgccagtagacgtcagtggcagttgcctcgcctatcggctaacaagttccttcggagtatataaatatagaatgtttacatactcctaagtttacttgcctccttcggagtatataaatatcccgaaggggaaggaggacgccagtggcagtggtaccgccactgcctgcttcctccttcggagtatgtaaaccccttcgggcaactaaagtttatcgcagtatataaatataggcagttggcaggcaactgccactgacgtcctattttaatactccgaaggaggcagttggcaggcaactgccactgacgtcccgtaagggtaaggggacgtccactggcgtcccgtaaggggaaggggacgtaggtacataaatgtgctaggtaactaacgtttgattttttgtggtataatatatgtaccatgcttttaatagaagcttgaatttataaattaaaatatttttacaatattttacggagaaattaaaactttaaaaaaattaacatatg